(19) United States
(12) Patent Application Publication
(10) Pub. No.: US 2015/0306108 A1
(43) Pub. Date: Oct. 29, 2015

(54) COMPOSITIONS OF ALDH1A1 INHIBITORS AND METHODS OF USE IN TREATING CANCER

(71) Applicant: Indiana University Research & Technology Corporation, Indianapolis, IN (US)

(72) Inventors: Thomas D. Hurley, Indianapolis, IN (US); Daniela Matei, Indianapolis, IN (US)

(21) Appl. No.: 14/610,106

(22) Filed: Jan. 30, 2015

Related U.S. Application Data
(60) Provisional application No. 61/933,970, filed on Jan. 31, 2014.

(51) Int. Cl. A61K 31/522; A61K 31/277; A61K 31/4184; A61K 31/519

(52) U.S. Cl. CPC A61K 31/522; A61K 31/519; A61K 31/277; A61K 31/4184

(57) ABSTRACT

The present invention provides inhibitors of ALDH1A1 activity which have substantially no effect on either ALDH2 or ALDH3A1. Compositions and methods of use, as well as methods to treat cancer using the ALDH1A1 inhibitors, are also provided.

### CROSS-REFERENCE TO RELATED APPLICATIONS

This application claims priority to U.S. Provisional Application No. 61/933,970, filed Jan. 31, 2014 which is incorporated by reference for all purposes.

### STATEMENT REGARDING FEDERALLY SPONSORED RESEARCH OR DEVELOPMENT

This invention was made with government support under AA018123 awarded by the National Institutes of Health. The government has certain rights in the invention.

### BACKGROUND OF THE INVENTION

The aldehyde dehydrogenase (ALDH) superfamily of enzymes primarily catalyze the NAD(P)⁺-dependent oxidation of an aldehyde to its corresponding carboxylic acid (Black, Hum. Genomics, 2009. 4(2): p. 136-42). The human genome has at least 19 ALDHs. A primary function of the ALDH1A subfamily (ALDH1A1, ALDH1A2, and ALDH1A3), whose members share over 70% protein sequence identity, is the oxidation of retinaldehyde to retinoic acid, a critical regulator in a number of cell growth and differentiation pathways. Other aldehydes also serve as substrates for ALDH1A1, including acetaldehyde during ethanol metabolism, 3,4-dihydroxyphenylacetaldehyde (DOPAL) in dopamine metabolism, and (±)-4-hydroxy-2E-nonenal (4-HNE), a toxic by-product of oxidative stress pathways.

ALDH1A1 has been associated with a number of diseases. Down-regulation of ALDH1A1 has been reported in Parkinson's disease, possibly due to the build-up of the neurotoxic aldehyde DOPAL in dopamine metabolism (Durrenberger, Parkinsons Dis, 2012. 2012: p. 214714; Galter, Neurobiol Dis, 2003. 14(3): p. 637-47). ALDH1A1 knockout mice are able to resist diet-induced obesity (Ziouzenkova, Nat Med, 2007. 13(6): p. 695-702), while rodents given the nonselective ALDH1A1 inhibitor citral also exhibit reduced weight gain (Ress, Toxicol Sci, 2003. 71(2): p. 198-206), indicating that ALDH1A1 is playing a role in obesity and/or adipogenesis. Up-regulation of ALDH1A1 is a biomarker for both normal and cancer stem cells, but the role of ALDH1A1 in establishing and/or maintaining stem cells is not known (Marcato, Cell Cycle, 2011. 10(9): p. 1378-84; Kastan, lood, 1990. 75(10): p. 1947-50; Jones, Blood, 1995. 85(10): p. 2742-6; Storms, Proc Natl Acad Sci USA, 1999. 96(16): p. 9118-23). Further, ALDH1A1 and ALDH3A1 have long been linked to cancer drug resistance due to their roles in the metabolism of the anticancer agent cyclophosphamide (Emadi, Nat Rev Clin Oncol, 2009. 6(11): p. 638-47).

Selective inhibitors of ALDH1A1 are needed to understand the role of this enzyme in both normal and disease processes. As recently reviewed by Ma and Allan, a number of ALDH family members have been associated with both normal stem cells and cancer stem cells (Ma, Stem Cell Rev 2011, 7, 292-306). The viability of the ALDH1A1−/− mice suggests that ALDH1A1 is non-essential or can be compensated for by other family members during growth and development (Levi, Blood 2009, 113, 1670-80). On the other hand, ALDH1A1 is considered a biomarker for lung, ovarian, prostate and a number of other cancers. Ovarian cancer cells form spheroids, cellular aggregates that aid metastasis (Ucar, Chem Biol Interact 2009, 178, 48-55; Landen, Mol Cancer Ther 2010, 9, 3186-99; Li, Lab Invest 2010, 90, 234-44; Sodek, Int J Cancer 2009, 124, 2060-70). Recently ALDH1A1 was shown to be upregulated in ovarian cancer spheroids (Condello, Oncogene 2014).

The development of compounds that selectively target ALDH1A1 has proven to be difficult as the ALDH superfamily of enzymes shares many common structural and mechanistic features. These members generally function as homodimers or homotetramers, with each subunit containing three structural domains, a catalytic domain, a cofactor binding domain, and an oligomerization domain. The NAD(P)⁺ binding domain is a Rossmann-fold, a nucleotide binding site that consists of two sets of parallel beta sheets and alpha helices. The Rossmann-fold structure motif is found in the NAD⁺ binding domains of multiple dehydrogenase families, including ALDHs, lactate dehydrogenases, alcohol dehydrogenases, and glyceraldehyde-3-phosphate dehydrogenase (Dixon, Enzymes. 3d ed. 1979, New York: Academic Press; Rossmann, Evolutionary and Structural Relationships among Dehydrogneases, in The Enzymes, P. D. Boyer, Editor. 1975, Academic Press: New York City. p. 61-102; Perez-Miller, Nat Struct Mol Biol, 2010. 17(2): p. 159-64). There are differences in the Rossmann fold between ALDH and other oxidoreductases that could possibly be exploited for the development of small molecule modulators of various ALDH isoenzymes compared to other NAD⁺-binding enzyme families (Liu, Z. J., et al., Nat Struct Biol, 1997. 4(4): p. 317-26). However, there exists much structural similarity in the NAD⁺-binding site within the ALDH family and the development of selective modulators that target this site may present difficulties.

A number of ALDH's, including ALDH1A1 also possess esterase activity. Based on the ALDH2 sequence, site-directed mutagenesis has shown that Cys-302 is the essential nucleophile for both the esterase and dehydrogenase reaction, with Glu-268 acting as the general base to activate Cys-302 (Farres, J., et al., Biochemistry, 1995. 34(8): p. 2592-8; Wang, Biochemistry, 1995. 34(1): p. 237-43). The proposed catalytic steps for both the dehydrogenase and esterase reactions have been recently reviewed (Koppaka, Pharmacol Rev, 2012. 64(3): p. 520-39), although minor details still need to be resolved including the roles of second sphere residues in assisting proton transfer to solvent (Gonzalez-Segura, J Mol Biol, 2009. 385(2): p. 542-57; Tsybovsky, Biochemistry, 2007. 46(11): p. 2917-29).

The use of common active site residues for the two reactions makes it likely that modulators of the esterase reaction would also modulate aldehyde oxidation activity. In support of this hypothesis, the ALDH2 activator Alda-1 activates both the esterase and dehydrogenase activity of the enzyme and daidzin inhibits both reactions (Chen, Science, 2008. 321(5895): p. 1493-5; Keung, Proc Natl Acad Sci USA, 1993. 90(4): p. 1247-51). An additional advantage of the esterase reaction is that it does not require the cofactor NAD⁺ to be present, and so allows the screen to be less influenced by compounds binding to this site.

The human ALDH1 family, which shares over 60% protein sequence identity, is a particularly difficult challenge for inhibitor development since it contains the highest number of orthologs in the genome (ALDH1A1, ALDH1A2, ALDH1A3, ALDH1B1, ALDH1L1, ALDH1L2, and ALDH2). Compounds such as diethylaminobenzaldehyde (DEAB) and disulfiram are potent inhibitors of ALDH1A1, with IC₅₀'s in the nM range, but both also inhibit ALDH2 (Koppaka, Pharmacol Rev, 2012. 64(3): p. 520-39; Moreb, Chem Biol Interact, 2012. 195(1): p. 52-60). DEAB is also a relatively potent inhibitor for a number of other ALDH1 family members, although not ALDH1L1 (Morgan, Chemico-Biological Interactions, 2014).

ALDH1A1 and ALDH3A1 are both involved in the metabolism of the cancer drug cyclophosphamide, metabolizing the active compound to a less active form and contributing to drug resistance. ALDH3A1 inhibitors have been shown to increase sensitivity to the cyclophosphamide analog mafosphamide when combined in cell lines with high ALDH3A1 expression (Parajuli, B. J Med Chem 2014, 57, 449-61; Parajuli, Chembiochem 2014, 15, 701-12). ALDH1A1 could serve as a similar target to minimize cyclophosphamide resistance in cancers with high ALDH1A1 levels. The functional role that ALDH1A1 contributes to stem cells and cancer metastasis is not understood.

An in vitro high throughput screen (HTS) is one method of discovering novel, small molecule modulators for a particular enzyme. Typically, the rate of aldehyde oxidation by ALDH is studied by monitoring the formation of NADH at 340 nm on a spectrophotometer (molar extinction coefficient of 6220 M⁻¹ cm⁻¹) (FIG. 1A). However, this approach is not ideal for a screening assay as it is common for compounds in the libraries to absorb light in the same wavelength range as NADH and leads to interference in this analytical approach. Therefore, another assay design is needed for an ALDH1A1 HTS.

One approach is to couple aldehyde oxidation to a second reaction that can be monitored by either fluorescence or UV/Vis spectrophotometry. For example, the dehydrogenase activity of ALDH2 was coupled to the NADH-dependent reduction of resazurin to resorufin to discover the ALDH2 activator Alda-1. However, a second approach would be to use the inherent esterase activity of ALDH1A1 to identify modulators. The ALDH1A1 ester substrate para-nitrophenylacetate (pNPA) is hydrolyzed to p-nitrophenol, which absorbs light at 405 nm and can be monitored spectrophotometrically, with minimal interference from library compounds (FIG. 1B).

### SUMMARY OF THE INVENTION

The present invention provides a pharmaceutical composition comprising a therapeutically effective amount of an inhibitor of ALDH1A1 activity selected from the group consisting of the following compounds (or analogs thereof)

| | | | |
|---|---|---|---|
| CM001 1.1 μM | 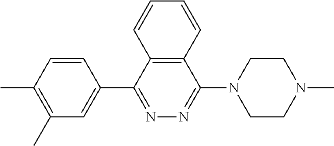 | CM045 2.5 μM | 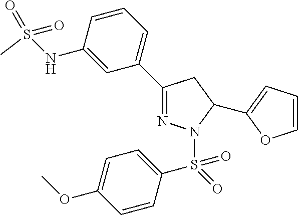 [SMILES: COc1ccc(S(=O)(=O)N2N=C(c3cccc(NS(C)(=O)=O)c3)CC2c2ccco2)cc1] |
| CM009 5.3 μM | 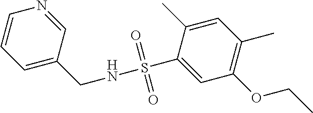 [SMILES: CCOc1cc(S(=O)(=O)NCc2cccnc2)c(C)cc1C] | CM047 0.31 μM | 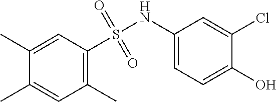 [SMILES: Cc1cc(C)c(S(=O)(=O)Nc2ccc(O)c(Cl)c2)cc1C] |
| CM010 1.3 μM | 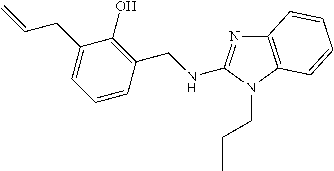 [SMILES: C=CCc1cccc(CNc2nc3ccccc3n2CCC)c1O] | CM053 0.21 μM | 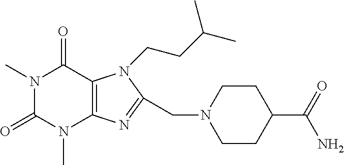 [SMILES: CC(C)CCn1c(CN2CCC(C(N)=O)CC2)nc2c1c(=O)n(C)c(=O)n2C] |
| CM020 0.45 μM | 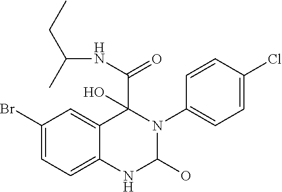 [SMILES: CCC(C)NC(=O)C1(O)c2cc(Br)ccc2NC(O)N1c1ccc(Cl)cc1] | CM055 0.24 μM | 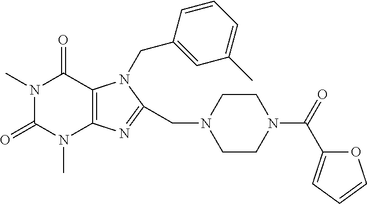 [SMILES: Cc1cccc(Cn2c(CN3CCN(C(=O)c4ccco4)CC3)nc3c2c(=O)n(C)c(=O)n3C)c1] |
| CM025 2.1 μM | 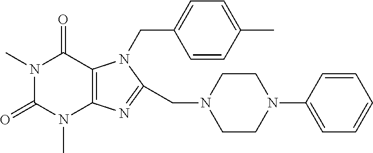 [SMILES: Cc1ccc(Cn2c(CN3CCN(c4ccccc4)CC3)nc3c2c(=O)n(C)c(=O)n3C)cc1] | CM056 5.4 μM | 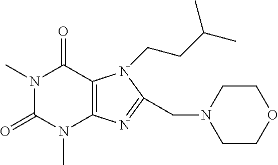 [SMILES: CC(C)CCn1c(CN2CCOCC2)nc2c1c(=O)n(C)c(=O)n2C] |
| CM026 0.80 μM | 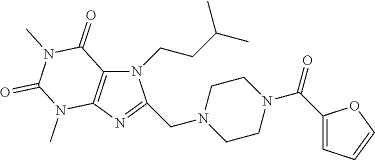 [SMILES: CC(C)CCn1c(CN2CCN(C(=O)c3ccco3)CC2)nc2c1c(=O)n(C)c(=O)n2C] | CM057 0.92 μM | 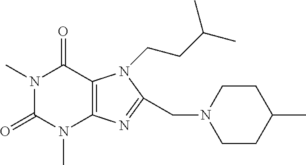 |
| CM028 2.0 μM | 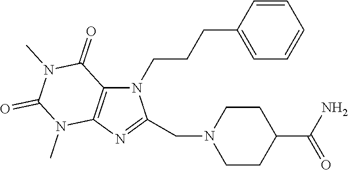 [SMILES: Cn1c(=O)c2c(nc(CN3CCC(C(N)=O)CC3)n2CCCc2ccccc2)n(C)c1=O] | CM302 1.1 μM | 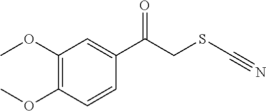 [SMILES: COc1ccc(C(=O)CSC#N)cc1OC] |
| CM037 4.6 μM | 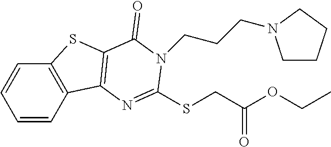 [SMILES: CCOC(=O)CSc1nc2c(sc3ccccc32)c(=O)n1CCCN1CCCC1] | CM306 3.5 μM | 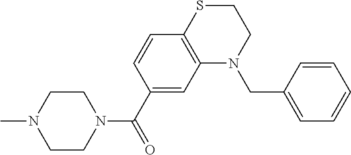 [SMILES: CN1CCN(C(=O)c2ccc3c(c2)N(Cc2ccccc2)CCS3)CC1] |
| CM038 0.26 μM | 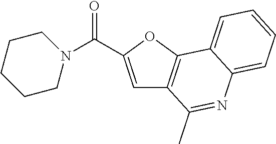 [SMILES: Cc1nc2ccccc2c2oc(C(=O)N3CCCCC3)cc12] | CM307 0.57 μM | 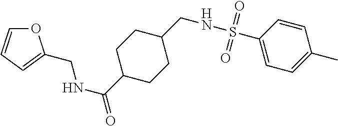 [SMILES: Cc1ccc(S(=O)(=O)NCC2CCC(C(=O)NCc3ccco3)CC2)cc1] |
| CM039 0.41 μM | 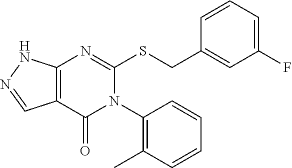 [SMILES: Cc1ccccc1-n1c(SCc2cccc(F)c2)nc2[nH]ncc2c1=O] | CM037a 23 μM | 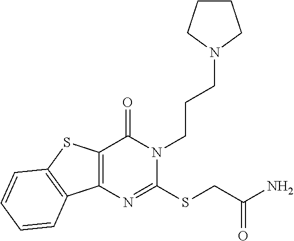 [SMILES: NC(=O)CSc1nc2c(sc3ccccc32)c(=O)n1CCCN1CCCC1] |
| CM037g 3.3 μM | 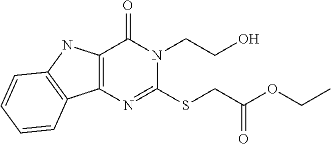 [SMILES: CCOC(=O)CSc1nc2c([nH]c3ccccc32)c(=O)n1CCO] | | |

or its pharmaceutically acceptable salt or a solvate thereof, and a pharmaceutically suitable carrier.

Specifically, in one embodiment of the composition, the ALDH1A1 inhibitor is

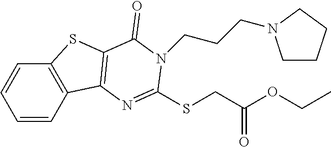 [SMILES: CCOC(=O)CSc1nc2c(sc3ccccc32)c(=O)n1CCCN1CCCC1]

(CM037) or an analog of CM037.

In alternative embodiment of the composition, the ALDH1A1 inhibitor is

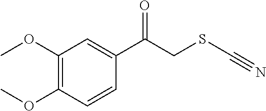 [SMILES: COc1ccc(C(=O)CSC#N)cc1OC]

(CM302) or an analog of CM302.

In alternative embodiment of the composition, the ALDH1A1 inhibitor is

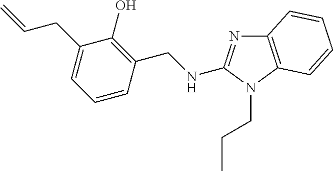 [SMILES: C=CCc1cccc(CNc2nc3ccccc3n2CCC)c1O]

(CM010), or an analog of CM010.

In one embodiment of the composition, the therapeutically effective amount ranges from about 0.001 μg/day/kg bodyweight to about 30 mg/day/kg bodyweight.

The invention also provides a method of treating cancer, the method comprising administering a therapeutically effective amount of a composition comprising an effective amount of an inhibitor of aldehyde dehydrogenase (ALDH1A1) to a subject in need thereof, wherein the cancer is treated. In one embodiment, the cancer to be treated includes ovarian, breast and lung cancer.

The invention also provides a method of inhibiting aldehyde dehydrogenase 1A1 (ALDH1A1) in a subject, the method comprising administering a therapeutically effective amount of a composition comprising an effective amount of an inhibitor selected from the group consisting of CM001, CM045, CM009, CM047, CM010, CM053, CM020, CM055, CM025, CM056, CM026, CM057, CM028, CM302, CM037, CM306, CM038, CM307, CM039, CM037a, CM037g as shown above, or analogs thereof. Kits for use of the composition and methods are also provided.

### DESCRIPTION OF THE FIGURES

The patent or application file contains at least one drawing executed in color. Copies of this patent or patent application publication with color drawings will be provided by the Office upon request and payment of the necessary fee.

FIG. 1A. Reactions used to discover ALDH1A1 modulators. NAD⁺-dependent aldehyde oxidation reaction monitored formation of NADH at 340 nm.

FIG. 1B. Reactions used to discover ALDH1A1 modulators. HTS used an NAD⁺-independent esterase reaction that monitored the formation of p-nitrophenol at 405 nm.

FIG. 2A. Structure of human ALDH1A1 (N121S) apo-enzyme. Ribbon representation of the structure of the homotetrameric ALDH1A1 with each monomer colored separately.

FIG. 2B. Structure of human ALDH1A1 (N121S) apo-enzyme. Ribbon representation of an ALDH1A1 monomer showing the location of cysteine 303 in the active site plus the location of ALDH1A1 N121S used for the HTS (PDB Code 4WJ9).

FIG. 3A. Structure of ALDH1A1 with reduced cofactor NADH. The location of the active site Cys-303 is shown in red. Surface rendition of NADH near the active site Cys-303.

FIG. 3B. Structure of ALDH1A1 with reduced cofactor NADH. Electron density maps of NADH with the original Fₒ-F_{c} in green contoured at 2.5 standard deviations and the final 2Fₒv-F_{c} map in grey contoured at 1.0 standard deviations.

FIG. 4. Overlap of the structure of ALDH1A1+NADH, in blue, with the structure of ALDH2+NADH, in grey.

FIG. 5. Z-factor determination for esterase screen. Each point represents the rate of change in absorbance at 405 nm of a reaction. The x-axis is the column (1-24) on the 384-well plate of the reaction. The blue data points represent the enzyme+substrate (ES) control, with an average value of 4.086; the red is enzyme+substrate+inhibitor, with an average value of 0.697; the open circles are the no enzyme control (blank). The lines represent 3× standard deviation from ES control (blue lines), ESI control (red lines), and blank (black lines). Each condition (ES, ESI, blank) performed on a separate plate with n=384.

FIG. 6. Representative plate from esterase HTS. Each point represents one well, with the x-axis the column (1-24) on the plate and the y-axis, the rate of change measured at wavelength 405 nm. Column 23 is the ES control, with an average value of 3.05 (n=16). Column 24 is the inhibition (ESI) control containing 25 μM Aldi-1. For this plate, an activator had a value ≧6.1 while an inhibitor had a value ≦1.22. Lines are 3× standard deviation, blue for ES and red for ESI. On this plate, we identified 3 activators (P14, N16, M19) and 1 inhibitor (D12) out of 352 compounds, with labeling based on their row and column on 384-well plate.

FIG. 7. The effect on dehydrogenase activity of 67 compounds identified via esterase HTS on three ALDH isoenzymes. The reactions used 20 μM compound and each bar represents mean/SEM (n=3). Only one compound for ALDH3A1 and two compounds for ALDH2 altered the respective activity of these enzymes more that 20%, while nearly half inhibited ALDH1A1 at least 50%.

FIG. 8A. Surface topography of the cofactor binding site for ALDH1A1. The orange sphere represents cations Yb present during crystallization.

FIG. 8B. Surface topography of the cofactor binding site for ALDH2 (PDB 1O02). The orange sphere in represent cations Mg present during crystallization.

FIG. 8C. Surface topography of the cofactor binding site for ALDH3A1 (PDB 4L2O).

FIG. 9A. Structure of CM026, a selective inhibitor of ALDH1A1.

FIG. 9B. Selectivity of 20 μM of CM026 with respect to nine ALDH isoenzymes.

FIG. 9C. IC₅₀ of CM026 with ALDH1A1.

FIG. 9D. Lineweaver-Burk representation of noncompetitive inhibition for CM026 (0-4 μM) verses varied acetaldehyde (100-800 μM) at fixed concentration of NAD⁺ (800 μM).

FIG. 9E. Lineweaver-Burk representation of uncompetitive inhibition for CM026 (0-3 μM) verses varied NAD⁺ (25-250 μM) at fixed concentration of propionaldehyde (200 μM). The IC₅₀ curves and Lineweaver-Burk plots represent one of three experiments performed for each condition, with each point the mean/SEM of three data points at each concentration.

FIG. 10A. Characterization of CM037, a selective inhibitor of ALDH1A1. CM037 has a molecular weight of 431.6 Daltons.

FIG. 10B. Selectivity of 20 μM of CM037 with respect to nine ALDH isoenzymes.

FIG. 10C. IC₅₀ of CM037 with ALDH1A1. The IC₅₀ curve represents one of three experiments performed, with each point the mean/SEM of three data points at each concentration.

FIG. 11A. Structure of ALDH1A1 N121S with CM026. CM026 binds in the active site near cysteine 303, shown in red. The location of Gly458 is shown in yellow.

FIG. 11B. Two-dimensional representation of the key hydrogen bonds, illustrated with red dashed lines, and hydrophobic interactions, illustrated with black arcs, between ALDH1A1 and CM026.

FIG. 11C. The electron density maps of CM026, with the original Fₒ-F_{c} map in green contoured at 2.5 standard deviations and the final 2Fₒ-F_{c} map in grey contoured at 1.0 standard deviations.

FIG. 12A. Structure of human ALDH1A1 with CM053. Two dimensional representation of the key hydrogen bonds, illustrated with red dashed lines, and hydrophobic interactions, illustrated with black arcs, between ALDH1A1 and CM053.

FIG. 12B. The electron density maps of CM053 with the original Fₒ-F_{c} map in green contoured at 2.5 standard deviations and the final 2Fₒ-F_{c} map in grey contoured at 1.0 standard deviations.

FIG. 13A. Structure of ALDH1A1 N121S with CM037. CM037 binds in the active site near cysteine 303, shown in red. The location of Gly458 is shown in green.

FIG. 13B. Two-dimensional representation of the hydrophobic interactions, illustrated with black arcs, between ALDH1A1 and CM037.

FIG. 13C. Binding of CM037 induces structural changes in ALDH1A1 (in blue) compared to apo-ALDH1A1 (in gray), particularly at W178. NADH binding (in cyan), induces conformational changes at the cofactor binding site, as seen here with E269.

FIG. 13D. The electron density maps of CM037, with the original Fₒ-F_{c} map in green contoured at 2 standard deviations and the final 2Fₒ-F_{c} map in grey contoured at 1.0 standard deviations.

FIG. 14A. Structural basis of selectivity of CM026 for ALDH1A1. Multiple sequence alignment in the region of ALDH1A1 Gly458 to the mature form of ALDH2.

FIG. 14B. Structure of ALDH1A1 with bound CM026 (blue) compared to ALDH2 (green) and ALDH3A1 (grey) indicating that a bulky amino acid such as the Asp of ALDH2 and Ile of ALDH3A1 would clash with CM026 and prevent the compound from inhibiting the enzyme. Sequence alignment was performed using NCBI delta-BLAST while structural alignment was performed using least square fit (LSQ) in Coot.

FIG. 15. Selectivity of compounds for WT vs G458N mutant. For CM026 and its analogs, 100 μM of compound was used. For CM037 and CM302, 20 μM of compound was used. Each value is mean/SEM (n=3).

FIG. 16. Comparison of the active site topography of human ALDH1A1, ALDH2, and ALDH3A1. The three isoenzymes were aligned using LSQ in Coot and the surface figures generated via Pymol. The active site cysteine is shown in red for all three isoenzymes. G458 in ALDH1A1 and its equivalent residues in ALDH2 (D457) and ALDH3A1 (1394) are shown in yellow.

FIG. 17. Comparison of the binding of CM026 to ALDH1A1 with the binding of daidzin to ALDH2. ALDH1A1 is shown in light blue with CM026 in dark blue. ALDH2 is shown in light green with daidzin in dark green. The active site cysteine is shown in red for both isoenzymes. Although both compounds bind in the same location, they bind in a different orientation enabling CM026 to inhibit ALDH1A1 but not ALDH2 due to steric hindrance of D474 (D457 in the mature sequence, in orange), while daidzin inhibits both isoenzymes. Daidzin-ALDH2 structure PDB 2VLE.

FIG. 18. Toxicity data showing effect of CM037. Single dose of CM037 was administered ip at 10, 30 and 60 mg/kg. Three mice were dosed accordingly for each dose and were observed for 3 weeks for behavior, eating, weight. Weights were monitored weekly. Blood counts and liver function tests were obtained 7-10 days after drug injection.

FIG. 19A. Gene Expression Analysis of Ovarian Cancer spheroids and monolayers. Morphology of ovarian cancer cells grown as spheroids and stained with methylene blue and fuchsin (400× magnification). Shown are spheroids derived from SKOV3, IGROV1, A2780 and primary human cells derived from ovarian cancer ascites. Arrows point to extracellular matrix deposited by SKOV3 cells and calcifications (psammoma bodies) formed in the ascites derived spheroids.

FIG. 19B. Gene Expression Analysis of ovarian cancer spheroids and monolayers. Hierarchical clustering displays differential expression profiles for IGROV1 cells grown as monolayer, spheroid, or spheroid to monolayer cultures (n=3 replicates). Rows represent individual samples and columns represent genes. Each cell corresponds to the level of expression of a particular gene in a given sample. A visual dual color code is utilized with red and blue indicating relatively high and low expression levels, respectively. The scale of color saturation, which reflects the gene expression levels, is included.

FIG. 19C. Differentially expressed genes between spheroids and monolayer were validated by semi-quantitative RT-PCR. Densitometry shows relative gene expression normalized for GAPDH.

FIG. 19D. Cell morphology of SKOV3 and IGROV1 cells grown as monolayers (m) and spheroids (s), 100× magnification (left panels). Semiquantitative RT-PCR assessed ALDH1A1 mRNA expression levels in SKOV3 and IGROV1 cells monolayers compared with spheroids (right panels).

FIG. 19E. Flow cytometry measures the Aldefluor positive cell population in SKOV3 and IGROV1 cells grown as spheroids compared with monolayers. DEAB-treated cells serve as negative controls. Measurements were performed in three replicates.

FIG. 20A. Gene networks in ovarian cancer monolayers versus spheroids. Gene networks generated using the IPA bioinformatics tool were ranked by log p-values and compared spheroid versus monolayer cultures. Networks with larger log p-values are more significant.

FIG. 20B. Analysis within the top ranked networks (log p value>25) displays interconnected genes as nodes. Genes are colored according to expression level values; red symbols correspond to up-regulated genes, while green symbols indicate down-regulation. Dashed lines between nodes show indirect interactions, while continuous lines indicate direct interactions.

FIG. 20C. Semiquantitative RT-PCR assessed mRNA expression levels for β-catenin and its targets (c-myc and cyclin D1) in SKOV3 and IGROV1 cells monolayers compared with spheroids.

FIG. 20D. SKOV3 and IGROV1 cells grown as monolayers were co-transfected with TCF/LEF1 luciferase reporter and Renilla control plasmid, prior to plating as monolayers or spheroids. Luciferase activity relative to renilla activity compared monolayers and spheroids at 24 and 48 hours and is expressed as fold increase. Data are shown as means of duplicate measurements+/−SD. Experiments were Experiments were repeated at least three times. Significant differences are marked.

FIG. 21A. β-catenin regulates ovarian cancer spheroid and tumor formation. SKOV3 cells were transfected with scrambled or β-catenin targeting siRNA prior to plating in ultra-low attachment plates. Morphology (left panels) and numbers (right panel) of spheroids derived from cells transfected with scrambled or β-catenin targeting siRNA. Sphere counts are shown as means+/−SD of quadruplicate measurements.

FIG. 21B. Semiquantitative RT-PCR measures β-catenin and c-Myc expression levels in SKOV3 spheroid cells transfected with scrambled or β-catenin targeting siRNA. Densitometry shows relative gene expression normalized for GAPDH.

FIG. 21C. Real-time PCR measures the expression levels of β-catenin target genes c-Myc and cyclin D1 in SKOV3 cells transfected with scrambled or β-catenin targeting siRNA. Data are shown as means+/−SD of 3 replicate measurements.

FIG. 21D. Western blotting shows β-catenin expression levels in SKOV3 cells stably transduced with control- and β-catenin targeting shRNA and used for ip inoculation of nude mice.

FIG. 21E. Tumor weight, volume, and numbers of peritoneal metastases derived from SKOV3 cells stably transduced with control- and β-catenin targeting shRNA and injected ip in nude mice (n=5 and 7, respectively). Data are shown as means+/−SEM. Significant differences are marked.

FIG. 22A. ALDH1A1 is a β-catenin target in ovarian cancer cells. Cell morphology of SKOV3 and IGROV1 cells grown as monolayers (m) and three spheroid generations (s1-s3, left panel). Semiquantitative RT-PCR for β-catenin and ALDH1A1 mRNA expression levels comparing monolayers and the three generation of spheroids (right panel).

FIG. 22B. Western blotting measures expression levels of β-catenin and cyclin D1 in monolayer cultures and three spheroid generations. Densitometry quantifies β-catenin, ALDH1A1, and cyclin D1 expression levels normalized for GAPDH.

FIG. 22C. Morphology of primary cells derived from ovarian cancer malignant ascites grown as monolayers (m), three spheroid generations (s1-s3), or spheroid to monolayer culture (s to m).

FIG. 22D. Semiquantitative RT-PCR for β-catenin and ALDH1A1 mRNA expression levels comparing monolayers, three generation of spheroids, and spheroid to monolayer cultures. Densitometry quantified β-catenin and ALDH1A1 normalized with the house-keeping gene 18S.

FIG. 22E. Flow cytometry quantifies Aldefluor positive cells derived from ovarian cancer ascites and grown as spheroids compared with monolayers. DEAB-treated cells serve as negative controls. Measurements were performed in duplicates.

FIG. 22F. Semiquantitative RT-PCR for ALDH1A1 expression levels in SKOV3 cells transfected with scrambled or β-catenin targeting siRNA.

FIG. 22G. Scheme representing the TCF/LEFT binding sequences within the ALDH1A1 promoter relative to the designed primers (top panel). ChIP assay used chromatin from IGROV1 cells immunoprecipitated with β-catenin or IgG (control). Results of PCR amplification are as follows: DNA ladder; chromatin from IGROV1 cells not subjected to IP (input) and amplified with 2 sets of primers corresponding to the two predicted TCF/LEF1 binding sequences on the ALDH1A1 promoter (lanes 1-2, f1/r1 and f2/r2) or with primers corresponding to the TCF/LEF binding site on the c-myc promoter (lane 3, positive control); chromatin immunoprecipitated with β-catenin antibody and amplified with ALDH1A1 promoter specific primers (lanes 4-5, f1/r1 and f2/r2); ALDH1A1 promoter nonspecific primers (lane 6, up f/r, negative control), or c-myc promoter specific primers (lane 7, positive control); or chromatin immunoprecipitated with IgG and amplified with ALDH1A1 and c-myc specific primers (lanes 8-10, negative controls).

FIG. 23A. Structure and properties CM037 {(ethyl-2-((4-oxo-3-(3-(pryrrolidin-1-yl)propyl)-3,4-dihydrobenzo[4,5]thioeno[3,2-d]pyrimidin-2-yl)thio)acetate)}.

FIG. 23B. Normalized residual activity of selected ALDH isoenzymes in the presence of 20 μM CM037 in the presence of saturating concentrations of aldehyde substrate.

FIG. 23C. A representative Lineweaver-Burk plot for the non-linear fit to the competitive inhibition equation for CM037 inhibition of ALDH1A1 versus varied acetaldehyde.

FIG. 23D. Morphology and number of spheres formed by IGROV1 cells under low attachment culture conditions after treatment with DMSO (control), CM037 (50 μM), or DEAB (50 μM) for 7 days.

FIG. 23E. Percentage of viable cells under the same treatment conditions was measured by trypan blue exclusion. Data are shown as means+/−SD of 3 replicate measurements. Significant differences are marked.

FIG. 24A. Effects of CM037 in ovarian cancer cells. Aldefluor activity measured by flow cytometry in IGROV1 cells treated with control (DMSO and DEAB) and CM037 (1-50 μM) for 3 days.

FIG. 24B. Morphology after treatment with DMSO (control) and CM037 (1, 5, 10 and 50 μM) for 7 days. Data are shown as means of triplicate measurements+/−SD. Significant differences are marked.

FIG. 24C. Number of spheres after treatment with DMSO (control) and CM037 (1, 5, 10 and 50 μM) for 7 days. Data are shown as means of triplicate measurements+/−SD. Significant differences are marked.

FIG. 24D. Percentage of viable cells after treatment with DMSO (control) and CM037 (1, 5, 10 and 50 μM) for 7 days. Data are shown as means of triplicate measurements+/−SD. Significant differences are marked.

FIG. 25A. CM037 and its analogs. The effect on aldehyde oxidation activity of ten CM037 analogs on three ALDH1A isoenzymes. The reactions used 20 μM compound and each bar represents mean/SEM (n=3).

FIG. 25B. IC₅₀ Curves for CM037 and two of its analogs. The parent compound CM037, an ester, is shown in black (IC₅₀=4.6 μM). An ester analog CM037g, shown in red, is slightly more potent than the parent compound (IC₅₀=3.3 μM). The amide CM037a, shown in blue, is less potent (IC₅₀=23 μM). Each point represents the mean/SEM of three readings, with the IC₅₀ values the mean/SEM of three independent experiments.

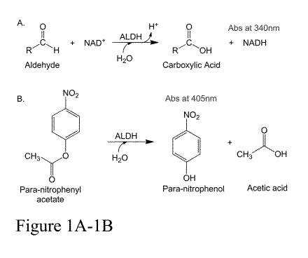

### DETAILED DESCRIPTION OF THE INVENTION

In General.

Before the present materials and methods are described, it is understood that this invention is not limited to the particular methodology, protocols, materials, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, cell lines, vectors, animals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

The Invention.

The invention provides a pharmaceutical composition comprising a therapeutically effective amount of an inhibitor of ALDH1A1 activity selected from the group consisting of the following compounds (or analogs thereof)

| | | | |
|---|---|---|---|
| CM001 1.1 μM | 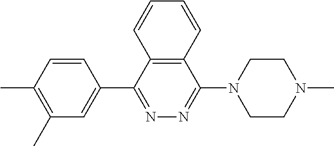 | CM045 2.5 μM | 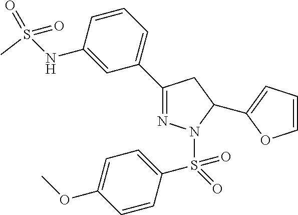 [SMILES: COc1ccc(S(=O)(=O)N2N=C(c3cccc(NS(C)(=O)=O)c3)CC2c2ccco2)cc1] |
| CM009 5.3 μM | 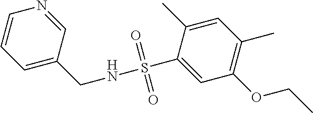 [SMILES: CCOc1cc(S(=O)(=O)NCc2cccnc2)c(C)cc1C] | CM047 0.31 μM | 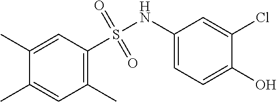 [SMILES: Cc1cc(C)c(S(=O)(=O)Nc2ccc(O)c(Cl)c2)cc1C] |
| CM010 1.3 μM | 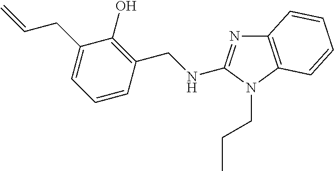 [SMILES: C=CCc1cccc(CNc2nc3ccccc3n2CCC)c1O] | CM053 0.21 μM | 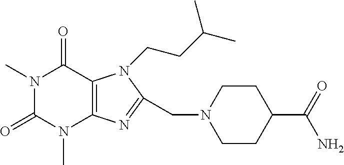 [SMILES: CC(C)CCn1c(CN2CCC(C(N)=O)CC2)nc2c1c(=O)n(C)c(=O)n2C] |
| CM020 0.45 μM | 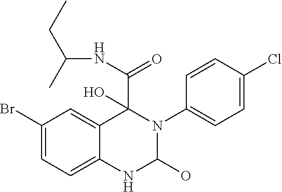 [SMILES: CCC(C)NC(=O)C1(O)c2cc(Br)ccc2NC(O)N1c1ccc(Cl)cc1] | CM055 0.24 μM | 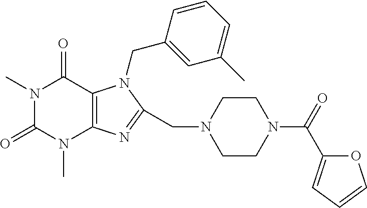 [SMILES: Cc1cccc(Cn2c(CN3CCN(C(=O)c4ccco4)CC3)nc3c2c(=O)n(C)c(=O)n3C)c1] |
| CM025 2.1 μM | 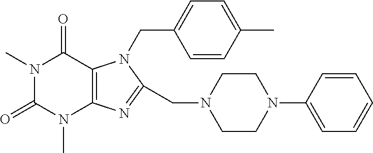 [SMILES: Cc1ccc(Cn2c(CN3CCN(c4ccccc4)CC3)nc3c2c(=O)n(C)c(=O)n3C)cc1] | CM056 5.4 μM | 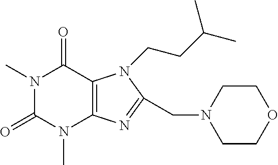 [SMILES: CC(C)CCn1c(CN2CCOCC2)nc2c1c(=O)n(C)c(=O)n2C] |
| CM026 0.80 μM | 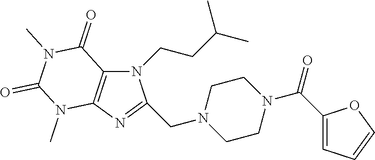 [SMILES: CC(C)CCn1c(CN2CCN(C(=O)c3ccco3)CC2)nc2c1c(=O)n(C)c(=O)n2C] | CM057 0.92 μM | 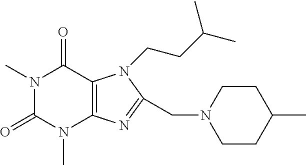 |
| CM028 2.0 μM | 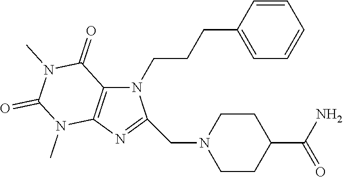 [SMILES: Cn1c(=O)c2c(nc(CN3CCC(C(N)=O)CC3)n2CCCc2ccccc2)n(C)c1=O] | CM302 1.1 μM | 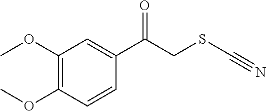 [SMILES: COc1ccc(C(=O)CSC#N)cc1OC] |
| CM037 4.6 μM | 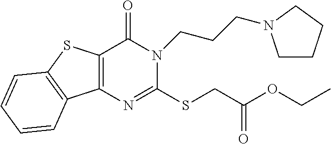 [SMILES: CCOC(=O)CSc1nc2c(sc3ccccc32)c(=O)n1CCCN1CCCC1] | CM306 3.5 μM | 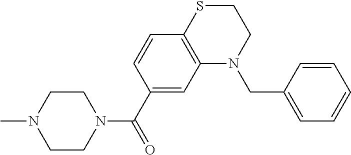 [SMILES: CN1CCN(C(=O)c2ccc3c(c2)N(Cc2ccccc2)CCS3)CC1] |
| CM038 0.26 μM | 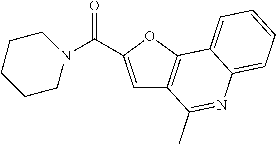 [SMILES: Cc1nc2ccccc2c2oc(C(=O)N3CCCCC3)cc12] | CM307 0.57 μM | 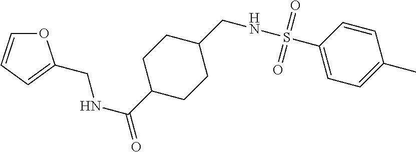 [SMILES: Cc1ccc(S(=O)(=O)NCC2CCC(C(=O)NCc3ccco3)CC2)cc1] |
| CM039 0.41 μM | 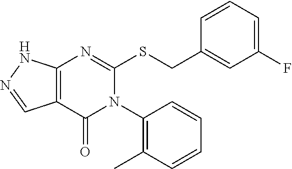 [SMILES: Cc1ccccc1-n1c(SCc2cccc(F)c2)nc2[nH]ncc2c1=O] | CM037a 23 μM | 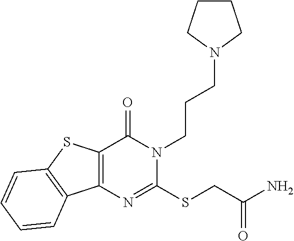 [SMILES: NC(=O)CSc1nc2c(sc3ccccc32)c(=O)n1CCCN1CCCC1] |
| CM037g 3.3 μM | 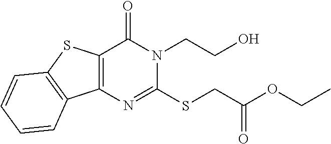 [SMILES: CCOC(=O)CSc1nc2c(sc3ccccc32)c(=O)n1CCO] | | |

or its pharmaceutically acceptable salt or a solvate thereof, and a pharmaceutically suitable carrier.

By “ALDH1A1 activity” we mean the inhibitors of the present invention inhibit at least 50% of ALDH1A1 activity at 10 uM, since it implies an IC50≦10 uM. We expect that dose-responses will eventually produce nearly 100% inhibition in the in vitro assays. We further expect the level of inhibition to vary depending on which cancer/disease is being treated, as determined by one of skill in the art, based on the level of ALDH1A1 present in the tumor and the ability of the tumor to dispose of the inhibitor.

In one embodiment, the composition has substantially no effect on ALDH2 or ALDH3A1. By “substantially no effect” we mean that composition comprising of the present invention will have 0 to about 5% inhibitory effect on ALDH2 or ALDH3A1. The more selective/specific the compound the fewer the anticipated side-effects.

In one embodiment, the composition is formulated in an oral, topical, transdermal, parenteral, injection or infusion dosage form.

In one embodiment, the therapeutically effective amount ranges from about 0.001 μg/day/kg bodyweight to about 30 mg/day/kg bodyweight. By “therapeutically effective amount”, we mean the composition includes an amount of the ALDH1A1 inhibitors that, when administered to a subject for treating a disease, is sufficient to effect the desired treatment for the disease. By “effective” we mean effective to for example, prevent the onset of the symptoms or complications, alleviate symptoms or complications, or eliminate the disease, condition, or disorder. An “effective” amount will prevent, alleviate, maintain or ameliorats any of the primary phenomena (initiation, progression, metastasis) or secondary symptoms associated with the disease. For example, effective treatment may kill diseased cells or reduce tumor size, inhibit tumor growth or metastasis, decrease tumor growth rate or metastasis rate, or maintain tumor size or the development of metastasis. The “therapeutically effective” or pharmaceutically effective” amount will vary depending on the compound, the disease state being treated, the severity or the disease treated, the age and relative health of the subject, the route and form of administration, the judgment of the attending medical practitioner, and other factors.

Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose.

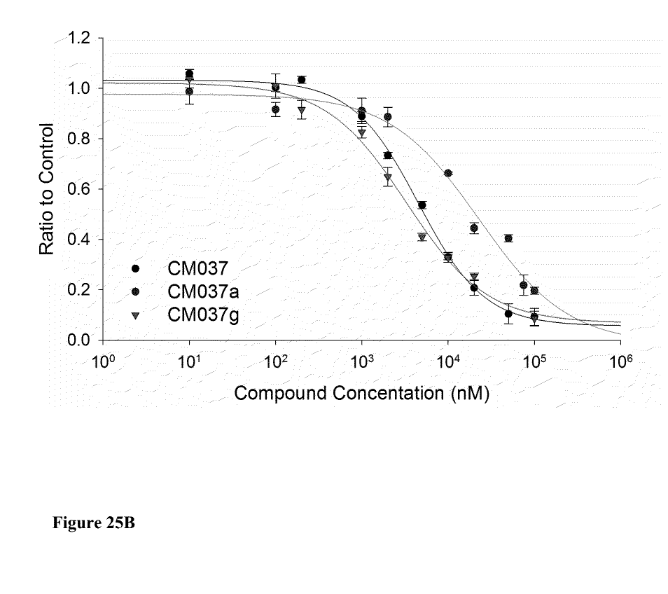

In one embodiment the ALDH1A1 inhibitor is CM037 or an analog of CM037 (see FIG. 25A-B). CM037 may be referred to as CAM-A37 or A37, which are equivalent to the CM037 above.

In alternative embodiments, the ALDH1A1 inhibitor is CM302 or CM010, or analogs of CM302 or CM010.

The invention also provides a method of treating cancer, the method comprising the step of administering a therapeutically effective amount a pharmaceutical composition comprising a therapeutically effective amount of an inhibitor of aldehyde dehydrogenase (ALDH1A1), or its pharmaceutically acceptable salt or a solvate thereof, and a pharmaceutically suitable carrier to a subject in need thereof, wherein the cancer is treated. In one embodiment, the ALDH1A1 inhibitor has substantially no effect on ALDH2 or ALDH3A1.

In one embodiment, the ALDH1A1 inhibitor is selected from the group consisting of CM001, CM045, CM009, CM047, CM010, CM053, CM020, CM055, CM025, CM056, CM026, CM057, CM028, CM302, CM037, CM306, CM038, CM307, CM039. CM037a or CM037g as shown above, or analogs thereof. By “analog” we mean a structure having at least about 80% similarity to the structures shown above, although in other embodiments an analog according to the present invention can have at least 90% similarity to the structures shown above.

In one embodiment, the cancer to be treated includes ovarian, breast and lung cancer.

In one embodiment, the ALDH1A1 inhibitor is formulated in an oral, topical, transdermal, parenteral, injection or infusion dosage form for administration to the subject. In one embodiment, the therapeutically effective amount ranges from about 0.001 μg/day/kg bodyweight to about 30 mg/day/kg bodyweight.

By “subject” we mean mammals and non-mammals. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex.

By “subject in need thereof” we mean an animal or human subject who is at risk of having cancer (e.g., a genetically predisposed subject, a subject with medical and/or family history of cancer, a subject who has been exposed to carcinogens, occupational hazard, environmental hazard) and/or a subject who exhibits suspicious clinical signs of cancer (e.g., blood in the stool or melena, unexplained pain, sweating, unexplained fever, unexplained loss of weight up to anorexia, changes in bowel habits (constipation and/or diarrhea), tenesmus (sense of incomplete defecation, for rectal cancer specifically), anemia and/or general weakness). Additionally or alternatively, the subject in need thereof can be a healthy human subject undergoing a routine well-being check up.

By “administering” or “administration” includes any means for introducing the ALDH1A1 inhibitors into the subject, preferably into the systemic circulation. Examples include but are not limited to oral, buccal, sublingual, pulmonary, transdermal, transmucosal, as well as subcutaneous, intraperitoneal, intravenous, and intramuscular injection. Any dosage effective to treat cancer is suitable for this invention. In one embodiment, the dosage ranges from about 0.001 μg to 10 μg per day per kg bodyweight. In other embodiments, the effective dosage ranges from about 0.0005 ug to 5 ug per day per kg bodyweight. In still other embodiments, the effective dosage ranges from about 0.1 ug to 30 mg per day per kg bodyweight.

By “treating” or “treatment”, we mean the management and care of a patient for the purpose of combating the disease, condition, or disorder. The terms embrace both preventative, i.e., prophylactic, and palliative treatment. Treating includes the administration of a compound of the present invention to prevent the onset of the symptoms or complications, alleviating the symptoms or complications, or eliminating the disease, condition, or disorder. Treatment also prevents, alleviates, maintains or ameliorates any of the primary phenomena (initiation, progression, metastasis) or secondary symptoms associated with the disease. For example, the treatment may kill diseased cells or reduce tumor size, inhibit tumor growth or metastasis, decrease tumor growth rate or metastasis rate, or maintain tumor size or the development of metastasis.

A compound is administered to a patient in a therapeutically effective amount. A compound can be administered alone or as part of a pharmaceutically acceptable composition. In addition, a compound or composition can be administered all at once, as for example, by a bolus injection, multiple times, such as by a series of tablets, or delivered substantially uniformly over a period of time, as for example, using transdermal delivery. It is also noted that the dose of the compound can be varied over time. A compound can be administered using an immediate release formulation, a controlled release formulation, or combinations thereof. The term “controlled release” includes sustained release, delayed release, and combinations thereof.

The present invention also provides a high throughput screen (HTS) assay for identifying compounds that modulate ALDH1A1 activity but have little to no effect on either ALDH2. The HTS of the present invention allowed us to minimize two potential problems: 1) identification of compounds that bind to the highly conserved cofactor site, and 2) monitor activity at a wavelength with minimal spectral overlap to that of the library compounds. These results indicate that this simple esterase-based in vitro assay of the present invention was successful in identifying novel, selective inhibitors of ALDH1A1. The high throughput screen was performed in 384-well, clear-bottomed plates, monitoring the change in absorbance of p-nitrophenol at 405 nm wavelength (molar extinction coefficient of 18000 M⁻¹ cm⁻¹) on a Spectramax plate reader. The 50 μL assay contained 730 nM ALDH1A1, 800 μM substrate para-nitrophenylacetate (pNPA), 10 μM of the chemical library compound, and 2% DMSO in 25 mM Na⁺-HEPES, pH 7.5 at 25° C. The non-selective ALDH1A1 inhibitor Aldi-1 at 25 μM final concentration was used as a positive control of ALDH1A1 esterase inhibition in each plate. Following a 2 minute incubation of enzyme and compound, the reaction was initiated by adding the substrate pNPA and the amount of p-nitrophenol produced was monitored for 7 minutes. Activity cut-offs were applied to the resulting data to select compounds for further characterization. For our purposes, an activator was defined as compounds that produced 2-fold or higher esterase activity compared to control, while an inhibitor was defined as compounds that produced reactions with 50% or less activity than control.

Kits. In an alternate embodiment of the invention, a kit for providing the composition of the present invention is provided. In one embodiment, the kit comprises an inhibitor according to the present invention, and instructions for use. Optionally the kit may include a pharmaceutically-acceptable carrier for use in combination with the inhibitor.

By “instructions for use” we mean a publication, a recording, a diagram, or any other medium of expression which is used to communicate the usefulness of the invention for one of the purposes set forth. The instructional material of the kit can, for example, be affixed to a container which contains the present invention or be shipped together with a container which contains the invention. Alternatively, the instructional material can be shipped separately from the container or provided on an electronically accessible form on a internet website with the intention that the instructional material and the ALDH1A1 inhibitor be used cooperatively by the recipient.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed description of the novel compounds and methods of the present invention are to be regarded as illustrative in nature and not restrictive.

### EXAMPLES

The following examples are, of course, offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims.

### Example 1

### Identification of ALDH1A1 Inhibitors

Materials.

All chemicals and reagents including para-nitrophenylacetate, propionaldehyde, NAD⁺, and buffers were purchased from Sigma Aldrich unless where noted otherwise.

Expression and Purification of ALDH Proteins.

ALDH1A1, ALDH2, and ALDH3A1 were prepared as described elsewhere. Protein used for kinetics was flash frozen in liquid nitrogen and stored at −80° C. ALDH1A1 protein used for X-ray crystallography was stored at −20° C. in a 50% (v/v) solution with glycerol and dialyzed against 10 mM Na⁺-ACES pH 6.6 and 1 mM dithiothreitol at 4° C. The ALDH1A1 protein used for the screen was produced from a cDNA obtained from Dr. Henry Weiner containing a known A-to-G SNP at position 72928972 on chromosome 9 (NCBI rs1049981), resulting in an Asn-to-Ser missense mutation at protein position 121. This SNP has been found in a small percentage of the HapMap-CEU population representing Utah residents with Northern and Western European ancestry, but there is no known clinical significance to the mutation.

The NCBI reference sequence for ALDH1A1 (wild-type) was constructed using the forward primer 5′-CTC TAT TCC AAT GCA TAT CTG AAT GAT TTA GCA GGC TGC ATC-3′ (NCBI Reference Sequence: NM_{—}000689.4) and its complement, using the QuikChange site-directed mutagenesis protocol. Unless where noted otherwise, ALDH1A1 WT protein was used for all aldehyde oxidation assays and the X-ray crystallography of the ALDH1A1-NADH structure. ALDH1A1-N121S was used for the HTS and the apo-enzyme structure.

For the kinetic assays, although the enzymes have more activity at a higher pH, a more physiologically relevant pH of 7.5 was used for both the HTS and dehydrogenase assays. This also kept the spontaneous hydrolysis of the ester substrate to a minimum and allowed direct comparison between the esterase and dehydrogenase assays.

Structural Determination of Human ALDH1A1.

For the apo-enzyme structure, crystals of ALDH1A1 N121S at 3-5 mg/mL concentration were equilibrated against a crystallization solution of 100 mM sodium BisTris, pH 6.2-7.0, 8-12% PEG3350 (Hampton Research), 200 mM NaCl, and 5-10 mM YbCl₃ at 25° C. Freezing of the crystals occurred in crystallization solution plus 20% (v/v) ethylene glycol. For the ALDH1A1-NADH structure, apo-enzyme crystals (WT) were prepared in the same manner as ALDH1A1 N121S crystals and were soaked for 2 hours with crystallization solution containing 1 mM NAD⁺. Freezing of the crystals occurred in crystallization solution with NAD⁺ plus 20% (v/v) ethylene glycol. Diffraction data was collected at Beamline 19-ID operated by the Structural Biology Consortium at the Advanced Photon Source, Argonne National Laboratory. Diffraction data were indexed, integrated, and scaled using either the HKL2000 or HKL3000 program suites. The CCP4 program suite was used for molecular replacement and refinement, using the sheep ALDH1 structure (PDB Code 1BXS) as a model for the apo-ALDH1A1 structure. The Coot molecular graphics application was used for model building and the TLSMD (Translation/Libration/Screw Motion Determination) server was used to determine dynamic properties of the protein.

Esterase Based High Throughput Screen on ALDH1A1.

The high throughput screen was performed in 384-well, clear-bottomed plates, monitoring the change in absorbance of p-nitrophenol at 405 nm wavelength (molar extinction coefficient of 18000 M⁻¹·cm⁻¹) on a Spectramax plate reader. The chemical library consisted of 64,000 compounds from ChemDiv Corp (San Diego, Calif.) at a final concentration of 10 μM. The 50 μL assay contained 730 nM ALDH1A1, 800 μM substrate para-nitrophenylacetate (pNPA), 10 μM compound, and 2% DMSO in 25 mM Na⁺-HEPES, pH 7.5 at 25° C. The non-selective ALDH1A1 inhibitor Aldi-1[33] at 25 μM final concentration was used as a positive control of ALDH1A1 esterase inhibition in each plate. Following a 2 minute incubation of enzyme and compound, the reaction was initiated by adding the substrate pNPA and monitored for 7 minutes. A Z-factor for the HTS was calculated by comparing the values of ALDH1A1 plus/minus Aldi-1 under the conditions of the HTS assay, each at n=384 to determine the quality of the HTS conditions.

A second control using no enzyme was also performed to determine whether our control inhibitor concentration had nearly 100% inhibition. An activator was defined as having 2-fold or higher esterase activity compared to control, while an inhibitor had 50% or less activity. After one round of screening, compounds identified as activators and inhibitors were rescreened using the same protocol and cutoffs to confirm the initial readings.

ALDH1A1, ALDH2, and ALDH3A1 Aldehyde Oxidation Activity Assays.

Hits from the HTS were ordered from ChemDiv to determine if they had any effect on aldehyde oxidation and whether they were selective for ALDH1A1 compared to ALDH2 and ALDH3A1. Dehydrogenase activity of the three isoenzymes were assayed by monitoring the production of NADH at 340 nm (molar extinction coefficient of 6220 M⁻¹ cm⁻¹) on a Beckman DU-640 or Cary 300 Bio UV-Vis spectrophotometer for 2 to 3 minutes. For ALDH1A1 and ALDH2, the reaction contained 100-200 nM enzyme, 200 μM NAD⁺, 100 μM propionaldehyde, and 1% DMSO in 50 mM Na⁺ BES, pH 7.5 at room temperature. For ALDH3A1, the reaction contained 20 nM enzyme, 200 μM NAD⁺, 300 μM benzaldehyde, and 1% DMSO in either 100 mM sodium phosphate or 50 mM Na⁺ BES at pH 7.5 at room temperature.

For most compounds, 20 μM concentration was used for the selectivity assays. However, due to solubility issues for CM307, 10 μM of compound was used. Following a 2 minute incubation of enzyme, compound, and NAD⁺, the reaction was initiated by adding substrate. For compounds with over 60% inhibition at 20 μM, IC₅₀ values for propionaldehyde oxidation were calculated by varying the concentration of the compounds from 0-200 μM under the same conditions as the selectivity assays. Data were fit to the four parameter EC₅₀ equation using SigmaPlot (StatSys v12.3).

Structure of Human ALDH1A1.

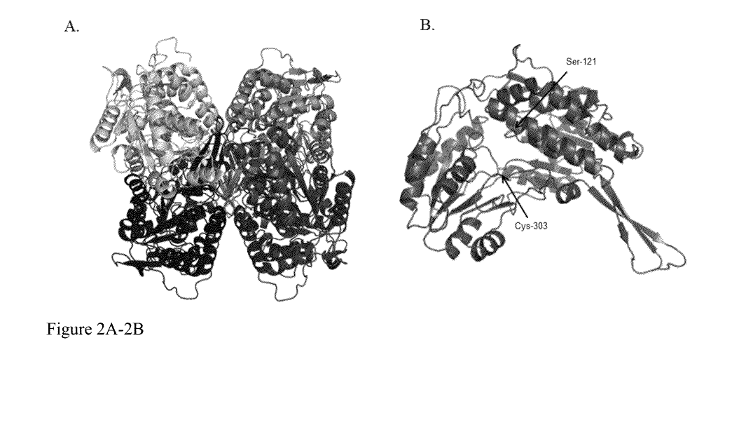

X-ray crystallography was used to compare the structure of human ALDH1A1 with other members of the ALDH enzyme superfamily. The structure of human ALDH1A1 had not been previously reported (FIG. 2 and Table 1, PDB Code 4WJ9). As expected, it is highly similar to both the human ALDH2 enzyme (PDB code 3N80), with which ALDH1A1 shares about 70% sequence identity, and the sheep ALDH1A1 (PDB code 1BXS), with over 90% sequence identity.

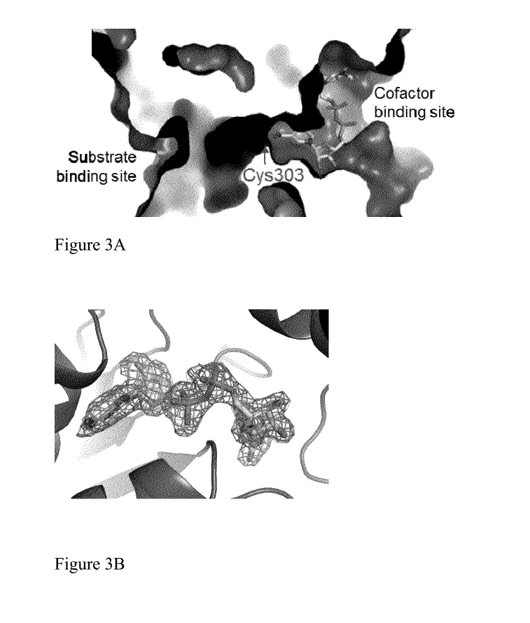

The structure of ALDH1A1 with NADH was determined to a resolution of 2.1 Å (FIG. 3 and Table 1, PDB Code 4WB9).

A comparison of the respective alpha-carbons in the structure of the N121S apo-enzyme and those of the wild-type ALDH1A1 structure complexed with NADH, generated an RMSD of 0.2 Å, consistent with a high degree of functional and structural similarity. The side chains of Ser and Asn both form similar hydrogen bonding interactions with Tyr297. Although wild-type apo-crystals were soaked with NAD⁺ the cofactor is bound in the hydrolysis position, characteristic of NADH binding, in the structure which suggests it could have been reduced via oxidation of PEG aldehydes.

The hydrolysis conformation observed here is similar to that seen in ALDH2 (PDB Code 1O02)[25] and the sheep ALDH1A1 (PDB Code 1BXS)[34] with cofactor, with the exception of the interaction of Glu-349 with a ytterbium cation bound to the pyrophosphate of NADH. Comparison of the structure of ALDH1A1, ALDH2, and ALDH3A1 (PDB Code 4L2O) with cofactor illustrates the difficulty of developing selective inhibitors for ALDH1A1 that target this site.

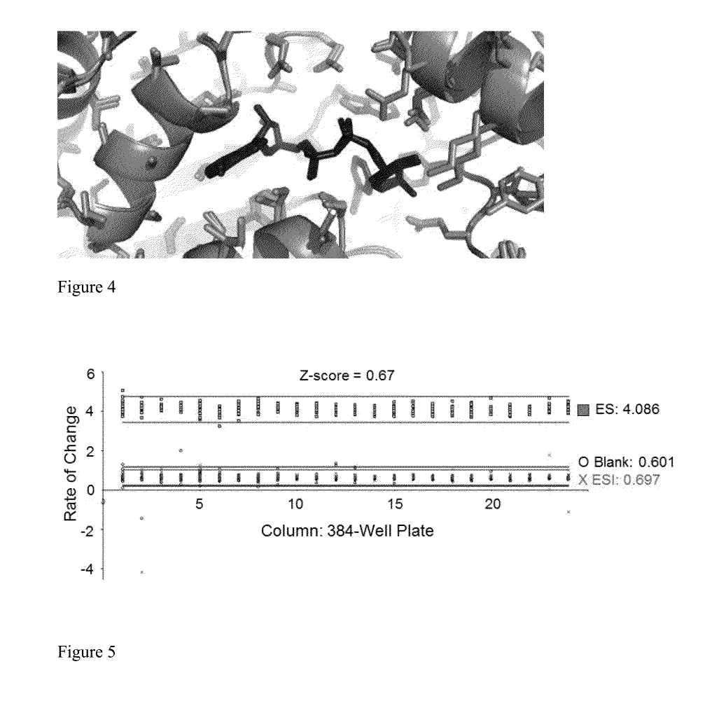

There is a high degree of similarity between the cofactor binding sites of ALDH1A1 and ALDH2 (FIG. 4), supporting use of an assay independent of the cofactor binding site in order to develop selective inhibitors for the ALDH1/2 class of enzymes. ALDH3A1 is the least similar both by structural topology and sequence identity, and as expected based on its ability to utilize both NAD⁺ and NADP⁺ these differences are most obvious near the adenosine ribose and pyrophosphate binding site.

TABLE 1
Data collection and refinement statistics of ALDH1A1-NADH.

| | Apo-ALDH1A1 | ALDH1A1-NADH |
|---|---|---|
| | N121S | Wild-type |
| Data Collection | (PDB 4WJ9) | (PDB 4WB9) |
| Space Group | P422 | P422 |
| Cell Dimensions | | |
| a, b, c [Å] | 109, 109, 83 | 109, 109, 83 |
| α, β, γ [°] | 90, 90, 90 | 90, 90, 90 |
| Resolution [Å] | 50-1.75 | 50-2.1 |
| R_{merge} | 0.056 (0.59) | 0.09 (0.52) |
| I/σᵢ | 31.5 (3.9) | 17.8 (4.9) |
| Completeness [%] | 99 (97) | 99 (100) |
| Redundancy | 9.6 (8.5) | 8.3 (7.9) |
| Refinement | | |
| No. of Reflections | 48862 | 29814 |
| R_{work}/R_{free} | 0.19/0.21 | 0.18/0.23 |
| No. of Atoms | | |
| Protein | 3839 | 3837 |
| Ligand/Ions | 2 | 50 |
| Water | 246 | 215 |
| R.M.S. Deviations | | |
| Bond Lengths [Å] | 0.010 | 0.017 |
| Bond Angles [°] | 1.21 | 1.8 |

Numbers in parenthesis represent value of highest resolution shell.

High Throughput Screen to Identify Modulators of ALDH1A1 Esterase Activity.

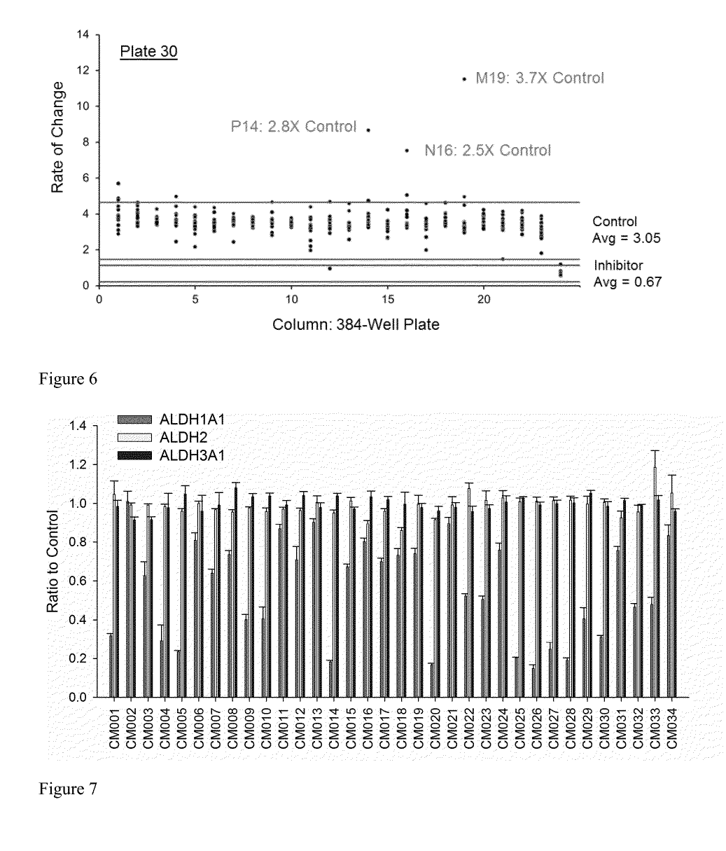

The Z-factor for the HTS comparing ALDH1A1 plus/minus inhibitor (Aldi-1) under screening conditions was 0.67 (n=384), indicating the screen is capable of identifying inhibitors from single assays. As shown in FIG. 5, there is a clear separation between the control reaction containing enzyme and substrate (ES Control) represented in blue, and the inhibitor control reaction containing enzyme, substrate plus an ALDH1A1 inhibitor (ESI control) represented in red. Also, the average value for ALDH1A1 with control inhibitor was similar to the no enzyme, blank control (mean rate of change of 0.70 vs 0.60), indicating our inhibition control (25 μM Aldi-1, IC₅₀=2.2 μM [33]) strongly inhibited ALDH1A1. For the HTS, we used an ALDH1A1 protein with a known SNP at residue 121. This N121S “mutant” is the open reading frame cloned by the Weiner group [26] and utilized for all their published work on ALDH1A1. The enzyme is active and behaved similarly to ALDH1A1 WT (Kₘ of 12 μM vs 15 μM, respectfully, with identical k_{cat}/Kₘ values at 2.7 min⁻¹·μM⁻¹ for the substrate propionaldehyde). The screen used a saturating amount of the esterase substrate pNPA (Kₘ=5 μM[35]). Each plate contained a control column with enzyme and substrate (ES control) and the average (n=16) of this intra-plate control served as the basis to determine whether a compound modified esterase activity.

An activator was defined as having 2-fold or higher esterase activity compared to this control, while an inhibitor had 50% or less activity. Each plate also contained a positive control for inhibition (ESI control) using the inhibitor Aldi-1. The initial round of the in vitro es terase-based screen of 64000 compounds yielded 631 compounds that activated ALDH1A1 and 278 compounds that inhibited ALDH1A1.

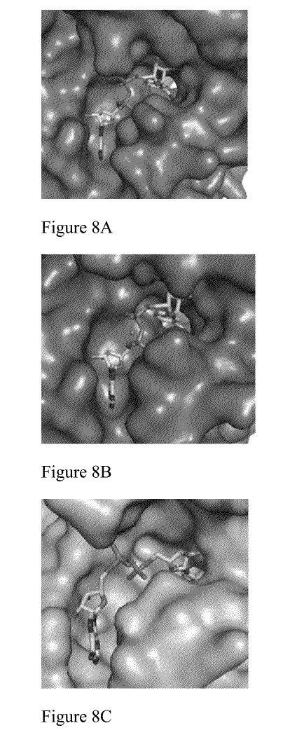

A sample plate from the first round of screening is shown in FIG. 6, illustrating 3 activators and 1 inhibitor out of 352 compounds tested. Following rescreening of the 909 compounds identified in the first round under identical conditions, nearly 75% did not meet these same selection criteria during the second, validation assay set. After two rounds, the esterase screen identified 241 activators and 15 inhibitors of ALDH1A1 esterase activity.

ALDH1A1, ALDH2, and ALDH3A1 Aldehyde Oxidation Activity Assays.

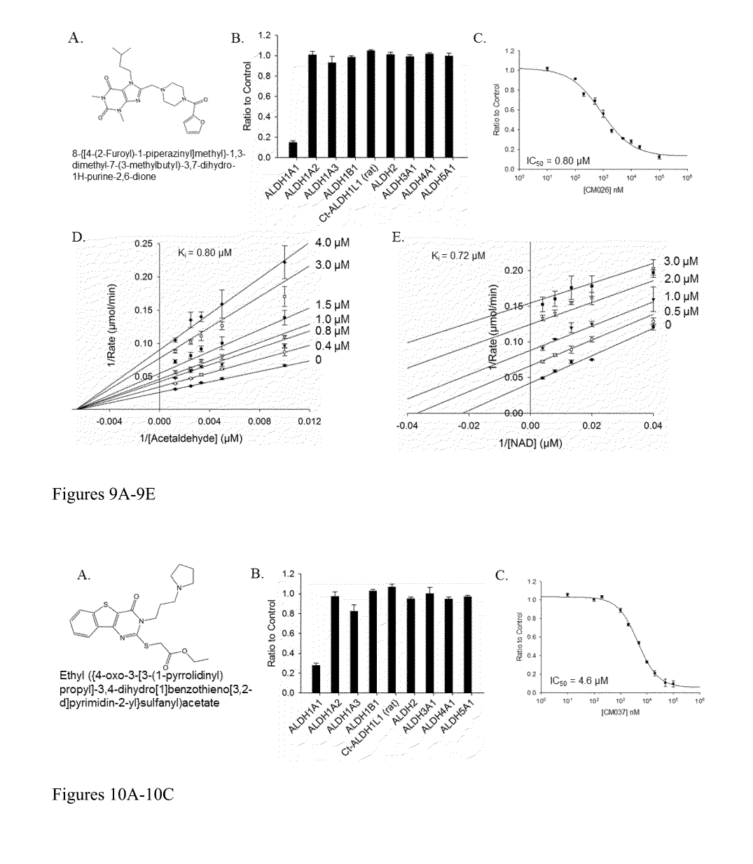

The 256 compounds were grouped based on structural similarities. From this set of compounds, we selected 57 esterase activators and 10 esterase inhibitors and tested their ability to alter aldehyde oxidation (FIG. 7). Of the 15 esterase inhibitors identified by HTS, only eight were commercially available.

However, close analogs of three were purchased and analyzed. Specifically, inhibitor 3343-2924 was substituted by 2188-3302 (CM310), inhibitor C699-0615 was substituted by C699-0244 (CM306), and inhibitor K788-2754 was substituted by K938-0803 (CM307). The effects the inhibitors have on aldehyde oxidation were tested using the standard assays performed in our laboratory to study these three ALDH isoenzymes.

For ALDH1A1 and ALDH2, 100 μM propionaldehyde is near to saturation (ALDH1A1 Kₘ ˜15 μM and ALDH2 Kₘ<1 μM). For ALDH3A1, the concentration of benzaldehyde used was set at its Kₘ. None of the 67 compounds tested activated aldehyde oxidation by ALDH1A1, ALDH2, or ALDH3A1 by more than 20%. However, of the 57 esterase activators examined at 20 μM concentration, 28 inhibited ALDH1A1 propionaldehyde oxidation by at least 50%.

Of the 10 esterase inhibitors tested at 20 μM concentration, four inhibited ALDH1A1 propionaldehyde oxidation by at least 50%, but two inhibitors (CM302 and CM303) also exhibited at least 50% inhibition of ALDH2 and therefore were not selective for ALDH1A1.

To a limited degree, CM302 also inhibited ALDH3A1 but none of the remaining 66 hits altered ALDH3A1 benzaldehyde oxidation more than 20% from control. Based on the selectivity assays of 67 esterase hits, 30 compounds selectivity inhibited ALDH1A1 compared to ALDH2 and ALDH3A1, while only two compounds inhibited both ALDH1A1 and ALDH2 at least 50% but not ALDH3A1.

IC₅₀ values were determined for compounds that inhibited propionaldehyde oxidation at least 60% at 20 μM concentration, with the most potent inhibitors and their IC₅₀ values shown in Table 2. Of the 57 esterase activators, 17 were structurally similar (CM022-031, CMOS 1-057) with all but one (CM024) inhibiting ALDH1A1 at 20 μM compound concentration. Based on IC₅₀ values, the most potent inhibitors selective for ALDH1A1 were CM038 and two structural analogs, CM053 and CM055, with all three hits having IC₅₀ values less than 300 nM. CM0302 was a potent inhibitor of both ALDH1A1 and ALDH2, with IC₅₀ values of 1.0±0.1 μM and 2.2±0.3 μM, respectfully.

To a limited extent, CM302 also inhibited ALDH3A1, but with an IC₅₀ value greater than 10-fold higher compared to ALDH1A1 and ALDH2. In comparison, the non-selective inhibitor Aldi-1, which was used as a control during the esterase HTS, has an IC₅₀ value of 2.2 μM. DEAB is a nonselective ALDH1 inhibitor used as a control for the ALDEFLUOR Assay (Stemcell Technologies, Vancouver, Canada), a flow cytometry assay commonly used to identify stem cells based on ALDH activity. DEAB has an IC₅₀ value of approximately 60 nM under these same conditions, but is also a potent inhibitor of other ALDH isoenzymes.

TABLE 2
IC50 values with ALDH1A1 for compounds that inhibit dehydrogenase
activity.

| Compound | |
|---|---|
| IC₅₀ [ μM] | Structure |
| CM001 1.1 ± 0.1* | 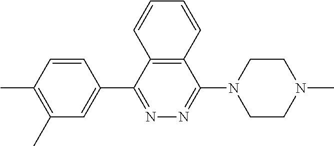 |
| CM009 5.3 ± 0.3* | 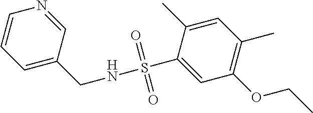 [SMILES: CCOc1cc(S(=O)(=O)NCc2cccnc2)c(C)cc1C] |
| CM010 1.3 ± 0.1* | 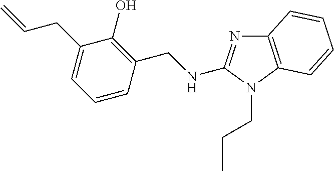 [SMILES: C=CCc1cccc(CNc2nc3ccccc3n2CCC)c1O] |
| CM020 0.45 ± 0.10 | 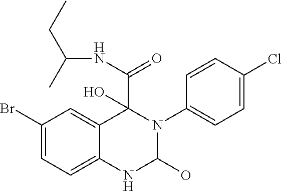 [SMILES: CCC(C)NC(=O)C1(O)c2cc(Br)ccc2NC(O)N1c1ccc(Cl)cc1] |
| CM025 2.1 ± 0.7 | 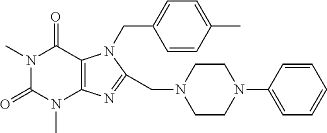 [SMILES: Cc1ccc(Cn2c(CN3CCN(c4ccccc4)CC3)nc3c2c(=O)n(C)c(=O)n3C)cc1] |
| CM026 0.80 ± 0.06 | 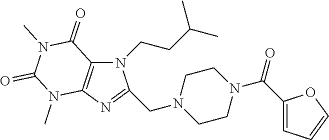 [SMILES: CC(C)CCn1c(CN2CCN(C(=O)c3ccco3)CC2)nc2c1c(=O)n(C)c(=O)n2C] |
| CM028 2.0 ± 0.1 | 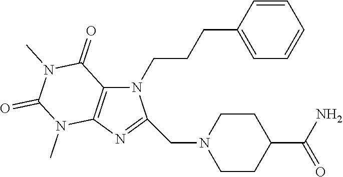 [SMILES: Cn1c(=O)c2c(nc(CN3CCC(C(N)=O)CC3)n2CCCc2ccccc2)n(C)c1=O] |
| CM037 4.6 ± 0.8 | 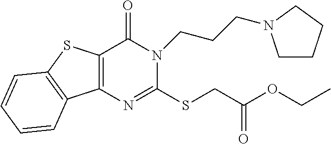 [SMILES: CCOC(=O)CSc1nc2c(sc3ccccc32)c(=O)n1CCCN1CCCC1] |
| CM038 0.26 ± 0.01 | 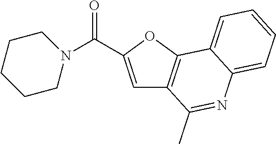 [SMILES: Cc1nc2ccccc2c2oc(C(=O)N3CCCCC3)cc12] |
| CM039 0.41 ± 0.01 | 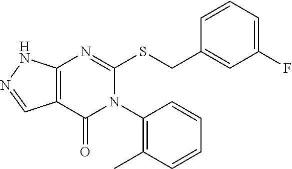 [SMILES: Cc1ccccc1-n1c(SCc2cccc(F)c2)nc2[nH]ncc2c1=O] |
| CM045 2.5 ± 0.5* | 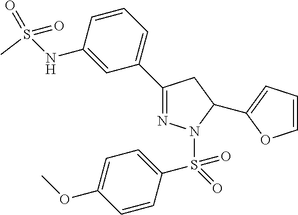 [SMILES: COc1ccc(S(=O)(=O)N2N=C(c3cccc(NS(C)(=O)=O)c3)CC2c2ccco2)cc1] |
| CM047 0.31 ± 0.03* | 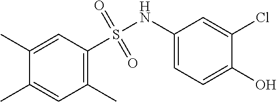 [SMILES: Cc1cc(C)c(S(=O)(=O)Nc2ccc(O)c(Cl)c2)cc1C] |
| CM053 0.21 ± 0.04 | 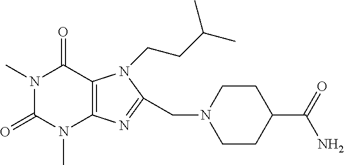 [SMILES: CC(C)CCn1c(CN2CCC(C(N)=O)CC2)nc2c1c(=O)n(C)c(=O)n2C] |
| CM055 0.24 ± 0.04 | 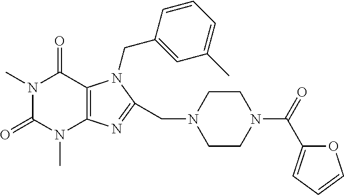 [SMILES: Cc1cccc(Cn2c(CN3CCN(C(=O)c4ccco4)CC3)nc3c2c(=O)n(C)c(=O)n3C)c1] |
| CM056 5.4 ± 0.8 | 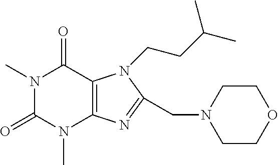 [SMILES: CC(C)CCn1c(CN2CCOCC2)nc2c1c(=O)n(C)c(=O)n2C] |
| CM057 0.92 ± 0.2 | 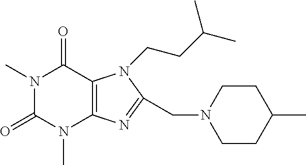 |
| CM302 1.1 ± 0.1 | 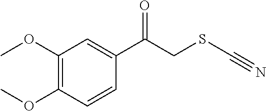 [SMILES: COc1ccc(C(=O)CSC#N)cc1OC] |
| CM306 3.5 ± 0.6 | 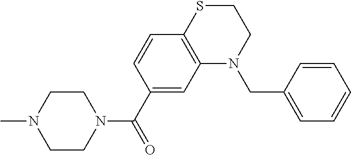 [SMILES: CN1CCN(C(=O)c2ccc3c(c2)N(Cc2ccccc2)CCS3)CC1] |
| CM307 0.57 ± 0.09 | 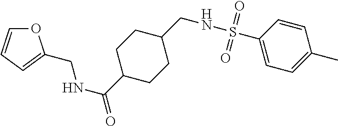 [SMILES: Cc1ccc(S(=O)(=O)NCC2CCC(C(=O)NCc3ccco3)CC2)cc1] |

Analogs for CM037A-I were also tested (see FIG. 25A-B).

Each value represents mean/SEM for three independent assays, each n=3. Values calculated using 100 μM Propionaldehyde and 200 μM NAD⁺. * Maximum inhibition<70%.

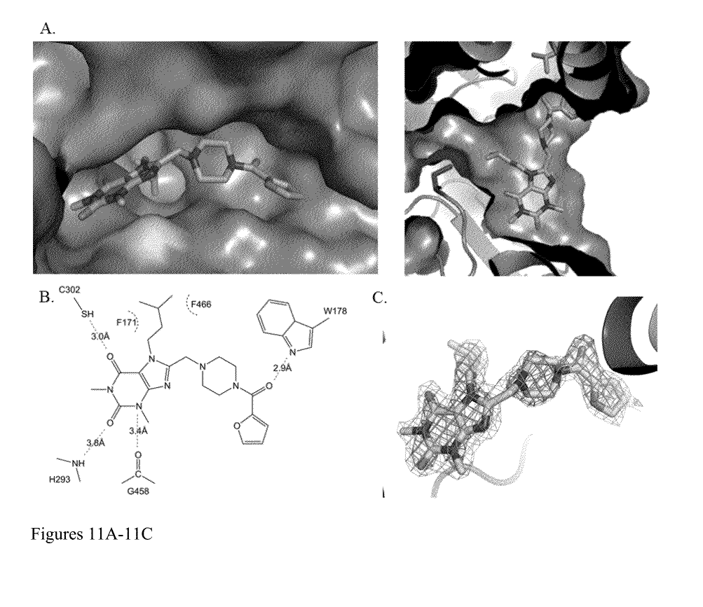

Comparison of the structures of human ALDH1A1, ALDH2, and ALDH3A1 indicate they exhibit a high degree of structural similarity, but demonstrate distinct differences within their substrate binding sites. In contrast, their respective coenzyme binding sites are less dissimilar, especially between ALDH1A1 and ALDH2 (FIG. 4). This supports our screening approach to avoid identifying compounds that interact at this location, as they are less likely to be selective for ALDH1/2 class members. However, it might be possible to utilize this approach for inter-class selectivity (FIG. 8).

The esterase screen used in this study was modeled after a previously reported screen for ALDH3A1 inhibitors that successfully identified two classes of selective ALDH3A1 inhibitors capable of increasing mafosfamide sensitivity in cancer cells. By adapting this assay to ALDH1A1, we screened a library of 64,000 compounds. Following one round of screen, our assay identified over 900 compounds that modified ALDH1A1 esterase activity. Rescreening of these compounds under identical conditions resulted in 256 confirmed hits that modified ALDH1A1 esterase activity. Therefore, the effect on esterase activity of <30% of the identified activators/inhibitors identified in round one were successfully repeated in round two. Although these replicability results may seem low, HTS are inherently noisy to begin with, producing many false positives that are eliminated in the second round.

As shown in FIG. 5, simply calculating the Z-factor produced outliers despite identical conditions within one plate. Some reasons for poor replicability include inaccuracies in compound concentration, spectral interference from the compounds, errors in robot pipetting, and debris or bubbles in the well that interfered with the reading. The second round of screening is designed to remove these false positives from consideration, conserving both time and resources. Since the HTS identified 256 compounds, the large number of false positives from round one was not a concern.

We examined the effect on dehydrogenase activity of 67 of these compounds and found that 30 selectively inhibited ALDH1A1 compared to ALDH2 and ALDH3A1, while 2 inhibited both ALDH1A1 and ALDH2. Therefore, nearly 50% of the esterase modifiers identified also altered aldehyde oxidation and almost all of the compounds did so selectively for ALDH1A1 compared to two other ALDH's.

Of the 57 esterase activators tested, none activated the dehydrogenation reaction of ALDH1A1, but nearly half inhibited it. The esterase reaction is independent of NAD⁺, but the presence of either NAD⁺ or NADH will increase the rate of ester hydrolysis, depending on assay conditions. The substrate and cofactor binding sites are linked to the active site by a tunnel through the enzyme. For ester hydrolysis, the substrate can likely enter the active site via either end of this tunnel. To activate esterase activity, it is proposed that cofactor binding slows transit of the ester substrate out of the tunnel, increasing the number of productive encounters with the active site nucleophile and also possibly by directly activating the nucleophile (FIG. 2B).

Compounds that function as esterase activators but dehydrogenase inhibitors likely bind to the substrate-binding end of this tunnel. In a manner similar to activation via cofactor binding, compound binding slows the transit of pNPA out of the active site tunnel and increases the likelihood of a productive encounter with the active site cysteine. However, the effect these esterase activators have on the NAD⁺-dependent aldehyde oxidation reaction is the opposite. Binding of the compound along with cofactor binding alters access to the active site at both ends and therefore depending on the structure of the compound could inhibit dehydrogenase activity. However, as seen with the ALDH2 activator Alda-1, a compound that binds at the substrate binding end of the active site tunnel could also result in a dehydrogenase activator, depending on binding position, location relative to the active site residues and substrate size.

It is possible that a number of our esterase activators that had no effect on aldehyde oxidation acted at the cofactor binding site, activating the esterase reaction like NAD⁺/NADH. However, the levels of NAD⁺ used in the assays (approximately 4×K_{M}) might minimize their effect on aldehyde oxidation. If a compound did bind at the cofactor site, only an extremely potent or covalent modulator would be identified under these conditions.

Of the 241 esterase activators identified, 78 were structural analogs with a common xanthine ring core structure. Of these 78 compounds, 17 were tested (CM022-031, CM051-057) and 16 selectively inhibited dehydrogenase activity of ALDH1A1 with no effect on either ALDH2 or ALDH3A1. The esterase HTS also produced 8 other structural groups containing between 7 and 20 analogs each. As a consequence, 65% of the esterase hits could be classified into 9 structural groups (Table 3). There were an additional 8 structural classes containing between 2-6 analogs and 22 structurally unique compounds.

TABLE 3
Structural classes of hit compounds

| | | Dehydrogenase | |
|---|---|---|---|
| | HTS | Activity | |
| Structure | Hits | Tested | Results |
| 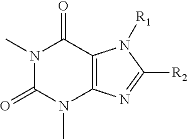 | 78 | 17 | 16 Inhib- itors |
| 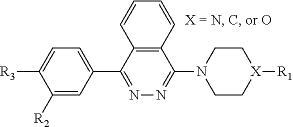 | 20 | 3 | CM001 |
| 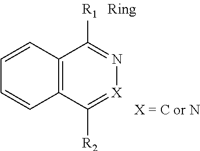 | 10 | 1 | No effect |
| 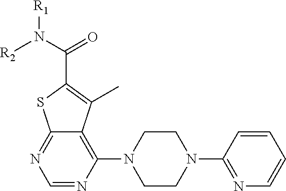 | 9 | 0 | |
| 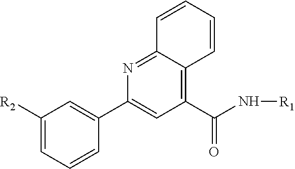 | 13 | 1 | No effect |
| 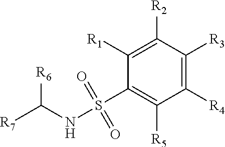 | 11 | 4 | 3 Inhib- itors |
| 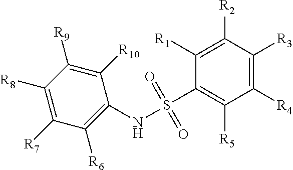 | 8 | 3 | CM047 |
| 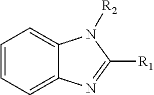 | 7 | 4 | CM010 |
| 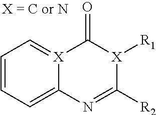 | 7 | 0 | |

Results of nine structural classes of esterase modulators representing 65% of the compounds identified from the esterase HTS.

CM037 was one of the structurally unique esterase activators that was found to be a potent and selective inhibitor of ALDH1A1 (IC₅₀=4.6±0.8 μM). A recent publication has shown that this compound, published as A37, is capable of disrupting spheroid formation in an ovarian cancer cell model by targeting ALDH1A1 activity. These results show that this esterase-based HTS identified a novel compound selective for ALDH1A1 compared to ALDH1A2, ALDH1A3, ALDH1B1, ALDH2, and ALDH3A1 and that this compound could enter a cell and alter a cancer phenotype by inhibiting ALDH1A1.

### Example 2

### CM037 Disrupts Spheroid Formation and Reduces Cell Viability

In this example, we show that our ALDH1A1-selective compound CM037 (also published as A37) can disrupt spheroid formation and reduce cell viability, supporting the hypothesis that ALDH1A1 is a target to improve cancer outcomes⁴¹.

Materials.

Reagents, including acetaldehyde, propionaldehyde, para-nitrophenylacetate, NAD⁺, and buffers were all purchased from Sigma Aldrich unless otherwise noted. Compounds were purchased from ChemDiv Corp. (San Diego, Calif.) and were >95% pure based on the NMR spectra provided by the vendor. Their chemical identities were verified by LC/MS in the Department of Chemistry, Indiana University-Purdue University Indianapolis and used without further purification.

ALDH Expression and Purification.

ALDH1A1, ALDH1A2, ALDH1A3, ALDH1B1, ALDH2, and ALDH3A1 were produced and purified as previously described. The ALDH1A1 protein used for the ALDH1A1-CM026 crystal contained an Asn-to-Ser SNP at residue 121. Unless where noted otherwise, ALDH1A1 WT protein was used for all aldehyde oxidation assays and for the ALDH1A1-CM053 structure. The full length cDNA for human ALDH4A1 and ALDH5A1 were generously provided by Dania Mochly-Rosen. ALDH4A1 was subcloned into the pET-28a expression plasmid and ALDH5A1 was in pTrcHis-Topo. The carboxyl terminal ALDH domain of rat ALDH1L1 was generously provided by Sergey Krupenko in the pRSET expression plasmid. ALDH1L1, ALDH4A1, and ALDH5A1 were expressed and purified as previously described for ALDH3A1³⁶ with the following modifications: 1) for ALDH1L1 and ALDH5A1 the medium contained 100 μg/mL ampicillin, 2) cells were lysed via 3 passages through a microfluidizer (DivTech Equipment), and 3) a single passage on a nickel-NTA column was used for purification, without the second Q-sepharose column used to purify ALDH3A1. Purified enzymes used for kinetics were stored at −80° C. ALDH1A1 used for crystallization was stored at −20° C. in a 50% (v/v) solution with glycerol and dialyzed against 10 mM Na⁺-ACES pH 6.6 and 1 mM dithiothreitol at 4° C.

Generation of Wild-Type ALDH1A1 and the G458N Mutant.

The QuikChange site-directed mutagenesis protocol was used to make point mutations. First, wtALDH1A1 was generated from the Weiner N121S polymorphism as previously described. A point mutation of wtALDH1A1 was performed to produce the G458N mutant. The ALDH1A1 G458N mutant was constructed using the forward primer 5′-GTG GGT GAA TTG CTA TAA CGT GGT AAG TGC CCAG-3′ and its complement. This G458N mutant was purified in the same way as other ALDH1A1 proteins (WT, N121S). G458N was stored at 2-mg/mL and 8-mg/mL at −80° C. Kinetic experiments for G458N were performed in the same manner as WT protein.

Aldehyde Dehydrogenase Activity Assay.

Dehydrogenase activity of purified recombinant ALDH1A1, ALDH1A2, ALDH1A3, ALDH1B1, ALDH2, and ALDH3A1 was assayed spectrophotometrically by monitoring the formation of NADH at 340 nm (molar extinction coefficient of 6220 M⁻¹ cm⁻¹) on a Beckman DU-640 or Cary 300 Bio UV-Vis spectrophotometer. Characterization of ALDH1A1 WT and ALDH1A1 G458N were performed by co-varying acetaldehyde and NAD⁺ concentrations in reactions containing 150-300 nM enzyme, 50-500 μM or 20-200 μM acetaldehyde, and 50-500 μM NAD⁺ in 50 mM sodium BES, pH 7.5 at 25° C. Reactions were initiated by adding enzyme. Selectivity of compounds for dehydrogenase activity of ALDH1A1, ALDH1A2, ALDH1A3, ALDH1B1 and ALDH2 were measured in a solution containing 100-200 nM enzyme, 200 μM NAD⁺, 1% DMSO, and 100 μM propionaldehyde (non-saturating for all enzymes tested, except ALDH2) in 50 mM sodium BES. ALDH3A1 activity was measured under the following conditions: 25 nM enzyme, 200 μM NAD⁺, 1% DMSO, and 300 μM benzaldehyde (˜K_{M}) in either 100 mM sodium phosphate buffer, pH 7.5 or 50 mM sodium BES, pH 7.5. All assays were performed at 25° C. and were initiated by the addition of the aldehyde substrate following a 2 minute pre-incubation with compound and cofactor. Selectivity was initially tested using 20 μM compound, with ALDH1A1-selective modulators further tested using 100 μM compound. IC₅₀ values for propionaldehyde oxidation were calculated by varying the concentration of the compounds from 0 to 200 μM. After a 2 minute pre-incubation with compound and NAD⁺, all reactions were initiated by the addition of propionaldehyde. Data were fit to the four parameter EC₅₀ equation using SigmaPlot (StatSys v12.3). The values represent the average of three independent experiments (each n=3). The mode of inhibition was determined via steady-state kinetics by co-varying inhibitor and substrate concentrations at fixed concentration of the second substrate. All reactions contained 100-150 nM ALDH1A1 and 1% DMSO in 50 mM sodium BES, pH 7.5 at 25° C. When cofactor NAD⁺ was varied, the reactions contained 200 μM propionaldehyde and 20-200 μM or 25-250 μM NAD⁺ (Kₘ=50 μM). When acetaldehyde was varied, the reactions contained 800-1000 μM NAD⁺ and 100-800 μM acetaldehyde (Kₘ=180 μM). All data were fit to competitive, noncompetitive, uncompetitive, and mixed inhibition models using both single substrate-single inhibitor or tight binding inhibition programs in SigmaPlot (StatSys v12.3). The appropriate model was selected through analysis of goodness-of-fit and the residuals of those fits. The values represent the average of three independent experiments (each n=3) using at least two protein preps.

Crystallization and Structure Determination of ALDH1A1 and its Complexes with Inhibitors.

Crystals of ALDH1A1 at 3-5 mg/mL concentration were equilibrated against a crystallization solution of 100 mM Na⁺ BisTris, pH 6.2-6.8, 8-12% PEG3350 (Hampton Research), 200 mM NaCl, and 5-10 mM YbCl₃ at 25° C. For the enzyme·CM026 and enzyme·CM037 complex, ALDH1A1·N121S was used, while wtALDH1A1 was used for the ALDH1A1·CM053 complex. CM026 and CM053 complexes were prepared by soaking crystals overnight with crystallization solution containing 500 μM inhibitor with 2% (v/v) DMSO. The CM037 complex was prepared by soaking crystals with crystallization solution containing 500 μM inhibitor, 1% (v/v) DMSO, and 1 mM NAD⁺ for 5 hours. Cryo-protection of the crystals for flash-freezing utilized 20% (v/v) ethylene glycol in the ligand soaking solution. Diffraction data were collected at Beamline 19-ID for the ALDH1A1-CM026 complex and Beamline 19-BM for the ALDH1A1-CM053 complex, both operated by the Structural Biology Consortium at the Advanced Photon Source (APS), Argonne National Laboratory. For the ALDH1A1-CM037 complex, data were collected at Beamline 23-ID-D (GM/CA), sponsored by National Institute of General Medical Sciences and National Cancer Institute of the National Institutes of Health at APS. All diffraction data were indexed, integrated, and scaled using either the HKL2000 or HKL3000 program suites. The CCP4 program suite was used for molecular replacement and refinement, using human apoALDH1A1 structure (PDB code 4WJ9) as a model for both structures. The molecular graphics application Coot was used for model building and TLSMD (Translation/Libration/Screw Motion Determination) server was used to determine the appropriate TLS tensors for refinement of the protein.

Kinetic Characterization of CM026 and CM037.

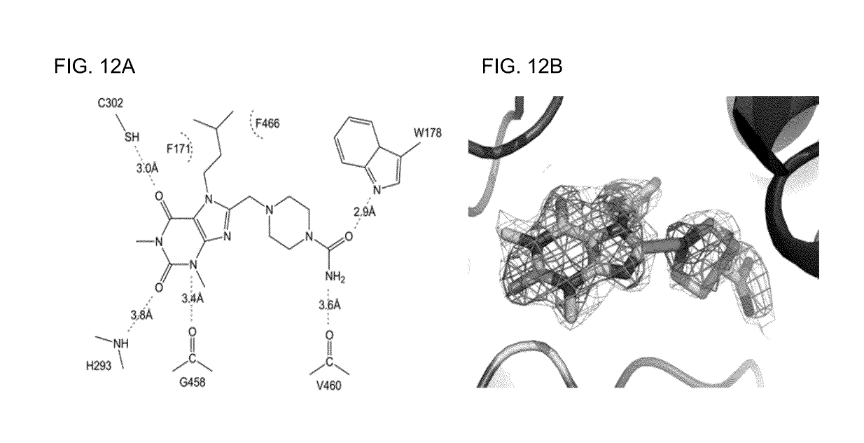

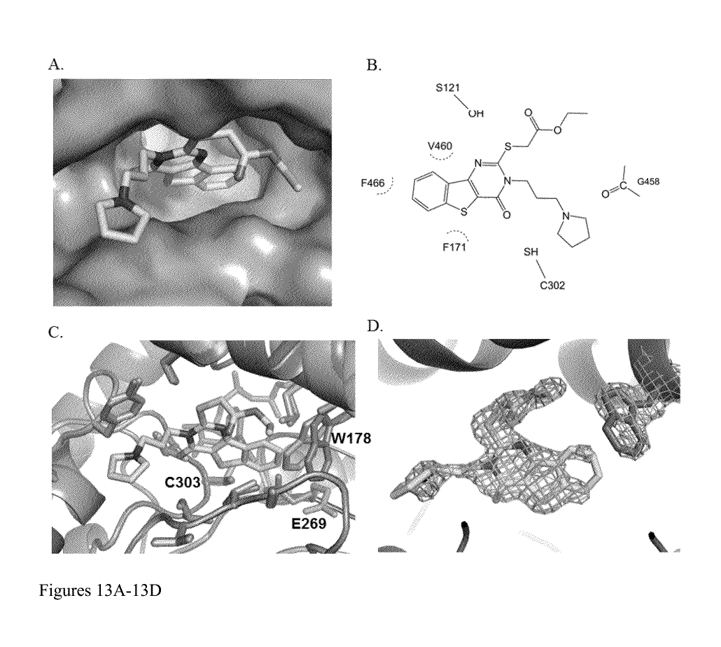

CM026 and CM037 emerged from the HTS as esterase modulators of ALDH1A1. CM026 (FIG. 9A) has a molecular weight of 442.5 Daltons and CM037 (FIG. 10A) has a molecular weight of 431.6 Daltons. These compounds have no structural similarity to any known, commercially available aldehyde dehydrogenase modulators. CM026 is a selective inhibitor for ALDH1A1 versus eight other ALDH isoenzymes examined. At a concentration of 20 μM, CM026 had no effect on seven other human ALDH isoenzymes (ALDH1A2, ALDH1A3, ALDH1B1, ALDH2, ALDH3A1, ALDH4A1, ALDH5A1) as well as the carboxyl-terminal ALDH domain of rat ALDH1L1 (FIG. 9B). At a concentration of 100 μM, CM026 modestly increased aldehyde oxidation catalyzed by ALDH1A2, ALDH1A3, and ALDH1B1. CM026 has good potency toward ALDH1A1 (IC₅₀ of 0.80±0.06 μM³⁹, FIG. 9C) for an initial hit compound. Complete inhibition of ALDH1A1 was not observed. CM026 has a noncompetitive partial mode of inhibition with respect to varied acetaldehyde, with a Kᵢ of 0.80±0.16 μM and =0.15±0.03, indicating maximum inhibition at 0.15 (Vₘₐₓ), (FIG. 9D). CM026 had an uncompetitive partial mode of inhibition with respect to varied NAD⁺ and exhibited a Kᵢ of 0.72±0.03 μM and =0.10±0.03 (FIG. 9E). It is possible that the size of the R-groups attached to the xanthine ring influences whether it is possible to slowly bind and release small aldehydes at the catalytic nucleophile, such substrate length dependency was also observed with the activator Alda-1 in ALDH2.

CM037 (FIG. 10A) was also selective for ALDH1A1 at 20 μM versus eight other ALDH isoenzymes tested (FIG. 10B). While 20 μM CM037 had little effect on most ALDH isoenzymes tested, ALDH1A3 was inhibited approximately 20% at this concentration. Higher concentrations were not tested due to solubility limits of CM037 under these assay conditions. CM037 exhibits an IC₅₀=4.6±0.8 μM toward ALDH1A1 versus the substrate propionaldehyde (FIG. 10C) and a competitive mode of inhibition with respect to varied substrate acetaldehyde and an average Kᵢ of 0.23±0.06 μM from three independent inhibition experiments. The effect on aldehyde oxidation activity of ten CM037 analogs on three ALDH1A isoenzymes is shown in FIG. 25A and FIG. 25B.

Structure Activity Relationship for Analogs of CM026.

There were 77 compounds in the initial screening hit list that were structurally similar to CM026. We have tested 17 members of this compound class and they exhibit good selectivity for ALDH1A1 compared to ALDH1A2, ALDH1A3, ALDH1B1, ALDH2, and ALDH3A1 (Table 4).

TABLE 4
SAR for CM026 Analogs.

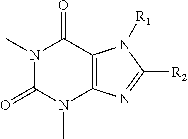

| | | | IC₅₀ in μM (SE) | | | | | |
|---|---|---|---|---|---|---|---|---|
| | R1 | R2 | ALDH1A1 | ALDH1A2 | ALDH1A3 | ALDH2 | ALDH1B1 | ALDH3A1 |
| CM022 | 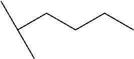 | 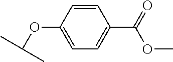 | 5.2* (0.8) | >100 | NI(A) | NI | NI | NI |
| CM024 | 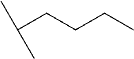 | 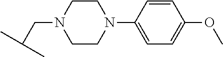 | >20 | NI | >100 | NI | >100 | NI |
| CM031 | 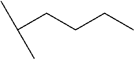 | 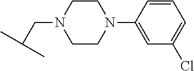 | >20 | >100 | >20 | >100 | >100 | NI(A) |
| CM053 | 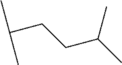 | 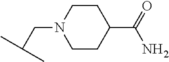 | 0.21 (0.04) | NI | NI(A) | NI | NI | NI(A) |
| CM026 | 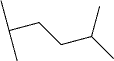 | 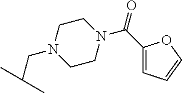 | 0.80 (0.06) | NI(A) | NI(A) | NI | NI(A) | NI(A) |
| CM057 | 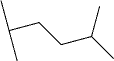 | 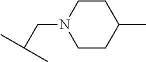 | 0.92 (0.2) | NI | >100 | >100 | >100 | NI |
| CM054 | 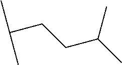 | 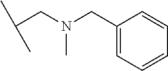 | 3.4* (0.7) | >100 | NI(A) | >100 | >100 | NI |
| CM056 | 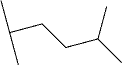 | 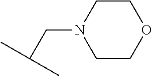 | 5.8 (1.2) | NI | NI | >100 | >100 | NI |
| CM030 | 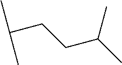 | 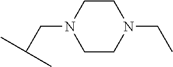 | >20 | NI | NI | NI | NI | NI |
| CM055 | 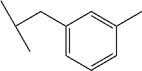 | 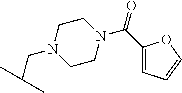 | 0.24 (0.04) | NI(A) | NI(A) | NI | NI | NI |
| CM029 | 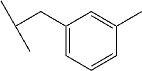 | 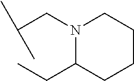 | 8.4 (1.0) | NI | NI(A) | NI | NI | NI |
| CM025 | 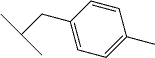 | 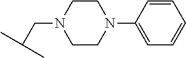 | 2.1 (0.7) | NI | NI(A) | NI | >100 | NI(A) |
| CM023 | 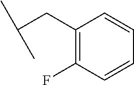 | 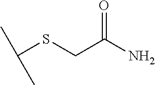 | 14* (2) | NI | NI(A) | NI | NI | >100 |
| CM051 | 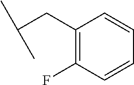 | 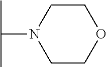 | >20 | NI(A) | NI(A) | NI | NI | NI |
| CM052 | 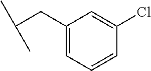 | 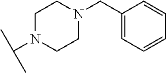 | >20 | >100 | NI(A) | NI | NI(A) | NI(A) |
| CM027 | 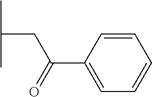 | 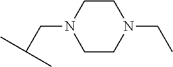 | 6.1 (1.1) | NI | NI | NI | NI | NI |
| CM028 | 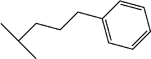 | 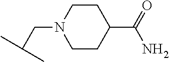 | 2.0 (0.1) | NI | NI | >100 | >100 | NI |

| | | | | | | | |
|---|---|---|---|---|---|---|---|
| Theophylline H | H | NI | NI | NI | NI | NI | NI |
| Caffeine CH₃ | H | NI | NI | NI | NI | NI | NI |

At 100 μM compound, NI stands for no inhibition and NI(A) indicates no inhibition but activation. * indicates <70% maximum inhibition.

At 100 μM, some compounds activated the aldehyde oxidation activity of other ALDH1 enzymes tested (eg. CM026), but at 20 μM, activation was less than 10%. These compounds share a common xanthine core structure with theophylline and caffeine, but neither of these stimulants affected ALDH1A1 activity at concentrations up to 250 μM, indicating that substituents at the R1 and R2 positions are necessary for ALDH1A1 inhibition (Table 4). Halogens on either R-group were not tolerated.

CM053 was the most potent analog examined, with an IC₅₀=210±40 nM and a Kᵢ=96±14 nM with noncompetitive mode of inhibition compared to varied substrate acetaldehyde. CM028 shares the same R2 group as CM053 but the isopentyl group at R1 has been replaced with a phenylpropyl group. CM028 is less potent, with an IC₅₀=2.0±0.1 μM, and exhibits a Kᵢ=240±40 nM with competitive mode of inhibition. The lower potency suggests that the phenylpropyl group might present steric conflicts not found with the smaller phenyl and isopentyl groups (Table 4). Unlike CM026, CM028 and CM053 demonstrated complete inhibition, which confirms that the nature of the R1 group alone does not influence the final extent of inhibition, since both CM026 and CM053 share the same R1 group.

Crystal Structure of ALDH1A1 Complexed with CM026, CM053 and CM037.

To determine the mechanism that underlies the ability of these compounds to selectively inhibit ALDH1A1, we used X-ray crystallography to determine the structure of the enzyme-compound complexes. We solved the crystal structures of human ALDH1A1 in complexes with CM026, CM053, and CM037 to resolutions between 1.80 Å and 1.95 Å (Table 5).

TABLE 5
Data collection and refinement statistics.

| | ALDH1A1- | ALDH1A1- | ALDH1A1- |
|---|---|---|---|
| | CM026 | CM053 | CM037 |
| Data Collection | PDB 4WP7 | PDB 4WPN | PDB 4X4L |
| Space Group | P422 | P422 | P422 |
| Cell Dimensions | | | |
| a, b, c (Å) | 109, 109, 83 | 109, 109, 83 | 109, 109, 83 |
| A, β, γ (°) | 90, 90, 90 | 90, 90, 90 | 90, 90, 90 |
| Resolution (Å) | 50-1.80 | 50-1.95 | 50-1.85 |
| R_{merge} | 0.082 (0.59) | 0.11 (0.66) | 0.058 (0.70) |
| I/σᵢ | 22.7 (4.7) | 18.3 (3.7) | 27.4 (3.3) |
| Completeness (%) | 99 (100) | 99 (100) | 99 (100) |
| Redundancy | 11.7 (11.7) | 9.3 (6.9) | 8.5 (8.8) |
| Refinement | | | |
| No. of Reflections | 44544 | 35048 | 40517 |
| R_{work}/R_{free} | 0.19/0.22 | 0.19/0.24 | 0.19/0.22 |
| No. of Atoms | 4109 | 4080 | 4066 |
| Protein | 3833 | 3858 | 3806 |
| Ligand/Ion | 35 | 33 | 80 |
| Water | 241 | 189 | 180 |
| R.M.S. Deviations | | | |
| Bond Lengths (Å) | 0.008 | 0.009 | 0.010 |
| Bond Angles (°) | 1.28 | 1.30 | 0.135 |

Numbers in parenthesis represent values of highest resolution shell.

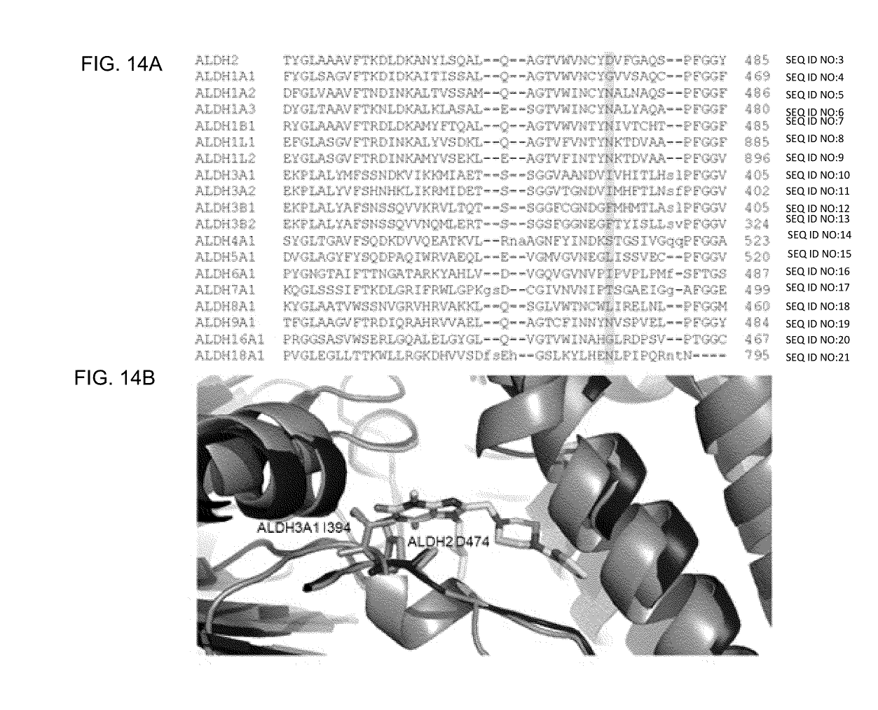

For CM026 and CM037, the naturally occurring N121S polymorphic variant of ALDH1A1 was used, while wtALDH1A1 was used for the structure with bound CM053. A comparison of the respective alpha carbons in the structure of the N121S·CM026 to those in the WT·CM053 generated an RMSD of 0.12Å, indicating a high degree of similarity between WT and the N121S mutant as expected since the two have very similar kinetic behavior. CM026 binds near the solvent exposed exit of the substrate-binding site (FIG. 11).

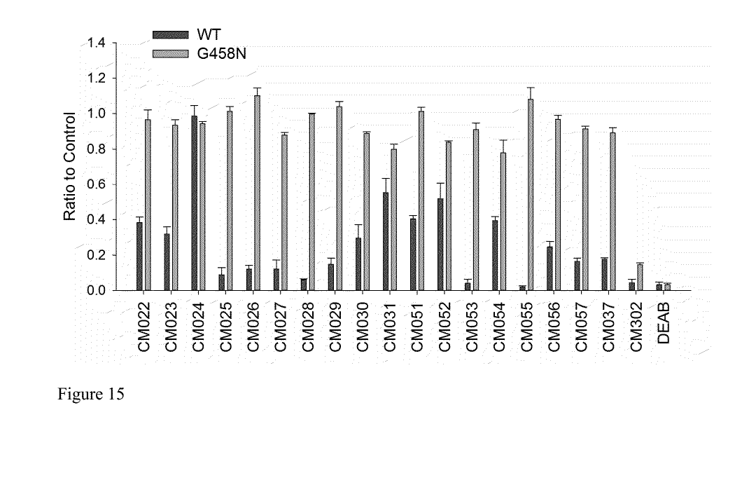

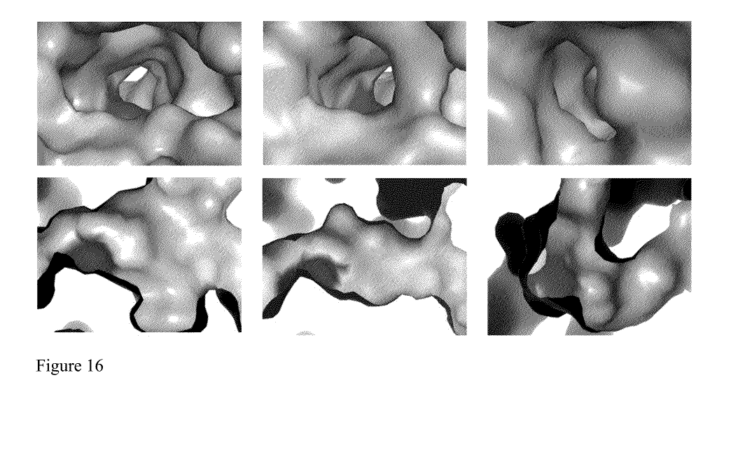

The xanthine rings for both CM026 and CM053 are parallel to and approximately 3.6 Å from Tyr297, with which it interacts via hydrophobic pi-stacking interactions. Four residues form hydrogen bonds with CM026; the xanthine ring interacts with His293, Cys302, and Gly458, while Trp178 interacts with the ketone group on R2. The isopentyl group of R1 projects towards Cys303 and fills much of the hydrophobic space bounded by Phe171 and Phe466. CM053 differs from CM026 only in its R2 group, which can form hydrogen bonds with two residues, Trp178 and Val460 (FIG. 12). CM037 binds at a similar location to the CM026 compounds, but its long axis is oriented almost orthogonal to that of CM026 and CM053 (FIG. 13A). Most of the tricyclic ring of CM037 is in a hydrophobic pocket formed by Phe171, Val460, and Phe466 with a potential hydrogen bond between the ring system's carbonyl oxygen atom and the side chain of Cys302 (FIG. 13B). The biggest structural adaptation to CM037 binding is the movement of Trp178 away from the substrate-binding site to accommodate the benzyl ring of CM037 (FIG. 13C). This conformational movement appears to be dynamic and impacts the observed electron density for both the benzyl group of CM037 and of Trp178. Trp178 is well ordered in all other structures determined of human ALDH1A, including our CM026 and CM053 structures, but has weak density for the benzyl moiety of the indole ring in this complex (FIG. 13D). Optimization of CM037 may be achieved by altering the thiophene and benzyl ring systems to alleviate these steric conflicts.

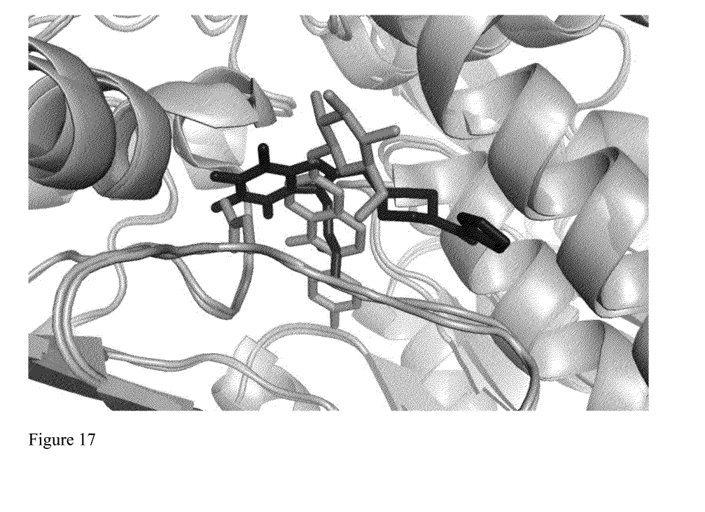

To better understand the selectivity of these compounds for ALDH1A1, we compared these structures against human ALDH2 (PDB code 1CW3), ALDH3A1 (PDB code 3SZA), and ALDH4A1 (PDB code 3V9G) and identified a critical glycine (Gly458) that is present near the xanthine ring binding site in ALDH1A1 [37, 42, 43]. This glycine is replaced by larger amino acid side chains in the other three human structures examined, as well as in sheep ALDH1A1 (PDB Code 1BXS). In rat ALDH1A2 (PDB code 1BI9), which shares 97% sequence identity to human ALDH1A2, this location is part of a small disordered loop not observed in the crystal structure [44, 45]. Using sequence alignments of the human genes, Gly458 in ALDH1A1 is replaced by an asparagine in ALDH1A2, ALDH1A3, and ALDH1B1, an aspartate in ALDH2, and an isoleucine in ALDH3A1 (FIG. 14A). As shown in FIG. 14B, these side chains would interfere with the position of the xanthine ring, effectively eliminating the ability of these analogs to bind to any isoenzyme but ALDH1A1.

Characterization of ALDH1A1 G458N Mutant.

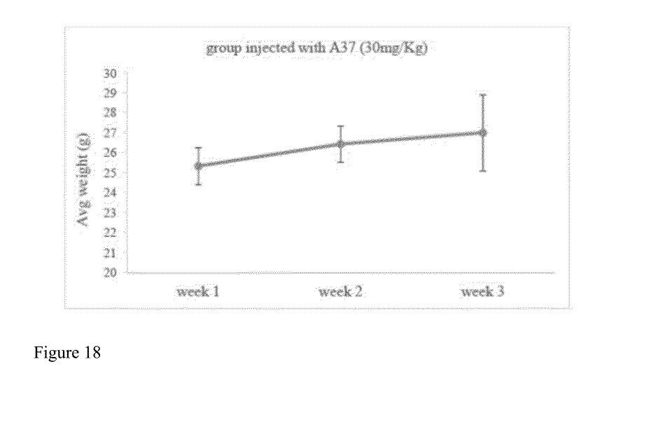

To confirm whether Gly458 in ALDH1A1 directly impacts the selectivity of the CM026 analogs and CM037 for ALDH1A1, we mutated the glycine at this position to asparagine, as found in ALDH1A2 and ALDH1A3. We determined the kinetic parameters for acetaldehyde oxidation for both the wild-type and G458N enzymes (Table 6). This mutation did not dramatically affect the enzyme's catalytic efficiency for aldehyde oxidation. However, when Gly458 is mutated to asparagine, CM026 no longer inhibits the enzyme at concentrations up to 100 μM and none of the CM026 analogs inhibited the mutant more than 25% (FIG. 15). Similarly, 20 μM CM037 no longer inhibited the G458N enzyme. However, the non-selective inhibitors DEAB and CM302 [39] both inhibit G458N, with IC₅₀ values of 0.52±0.10 μM and 3.1±0.3 μM respectively [39,46] (Table 6). These data support the hypothesis that the substrate-binding site, and in particular Gly458, determines the selectivity of both the CM026 and CM037 classes of compounds for ALDH1A1, and that bulkier side chains at this position in ALDH1A2, ALDH1A3, ALDH1B1, ALDH2, and ALDH3A1 occludes their binding to these ALDH isoenzymes.

TABLE 6
Kinetic parameters of ALDH1A1 WT and mutant G458N.

| | K_{M}^{Acetaldehyde} | k_{cat}/K_{M} | IC₅₀^{CM026} | IC₅₀^{CM037} | IC₅₀^{CM302} | IC₅₀^{DEAB} |
|---|---|---|---|---|---|---|
| | (μM) | (min⁻¹ · μM⁻¹) | (μM) | (μM) | (μM) | (μM) |
| WT | 177 ± 19 | 0.18 ± 0.02 | 0.80 ± 0.16 | 4.6 ± 0.8⁴¹ | 1.0 ± 0.1³⁹ | 0.057 ± 0.005⁴⁶ |
| G458N | 85.8 ± 1.6 | 0.21 ± 0.02 | NI | NI | 3.1 ± 0.3 | 0.52 ± 0.10 |

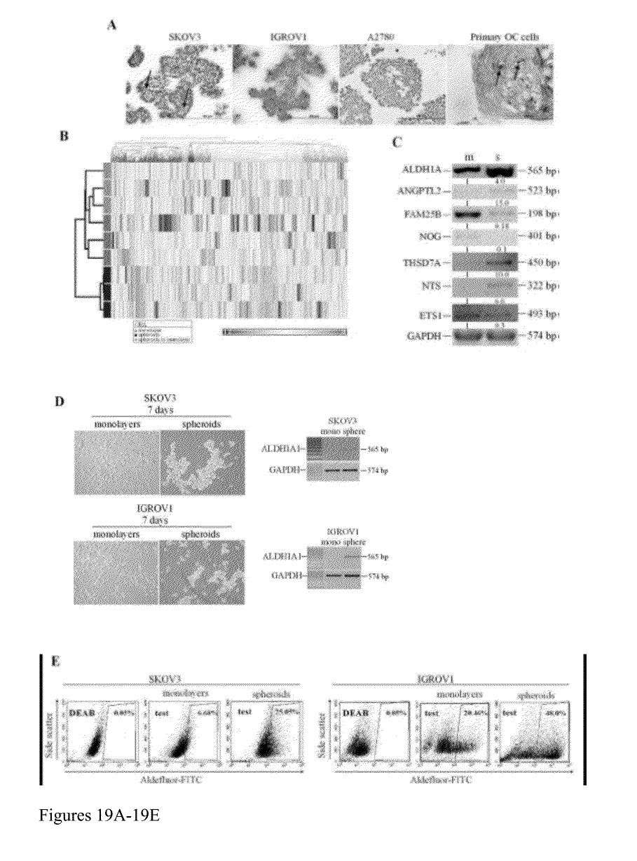

Although comparisons of available ALDH structures indicate a high degree of overlap, there exist distinct surface topographies that may enable development of selective inhibitors (FIG. 16).

ALDH1A1 possesses a wider opening leading to the active site, whereas ALDH2 has a much more constricted, cylindrical shaped site. ALDH3A1 possesses a wider inner vestibule near the catalytic nucleophile, with a much narrower and curved entryway. The narrower entries in ALDH2 and ALDH3A1 eliminate the binding site for the CM026 and CM037 classes of inhibitors in large part due to the side chain present at the position equivalent to Gly458. ALDH4A1 possess a serine at the position equivalent to Gly458. However the loop structure in which it resides is different enough from ALDH1A1 to prevent compound binding. Similar to daidzin, both the CM026 and CM037 classes of compounds are planar, multi-ringed structures that adopt binding modes that take advantage of the topological characteristics unique to the ALDH isoenzyme toward which they demonstrate selectivity and neither of these new compounds can be accommodated in the restricted substrate binding sites of ALDH3A1 or ALDH2.

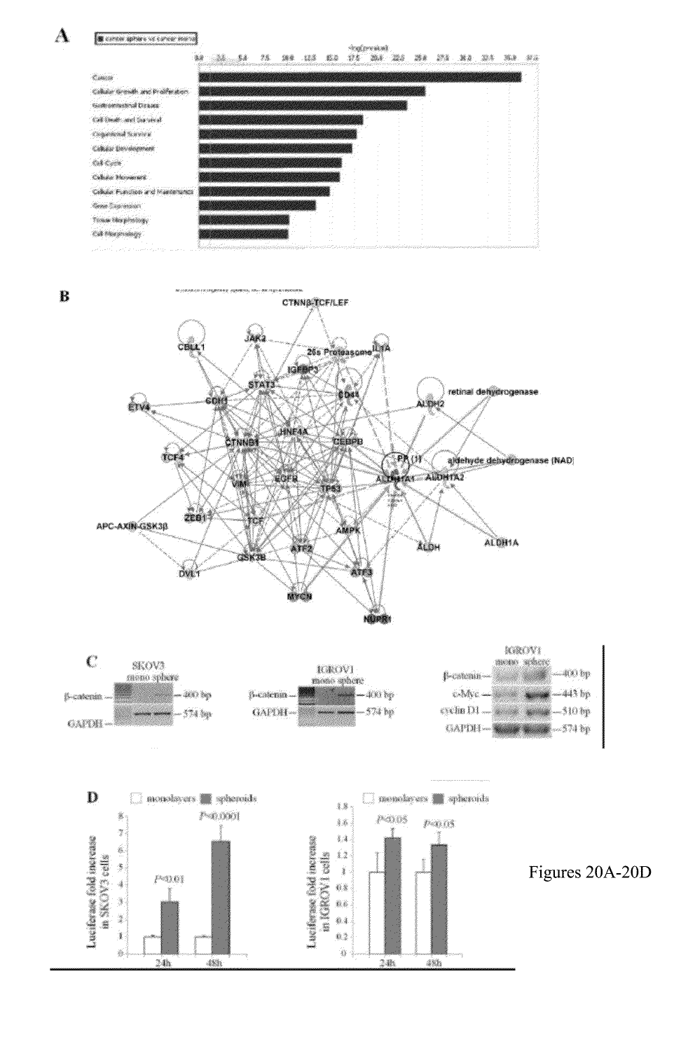

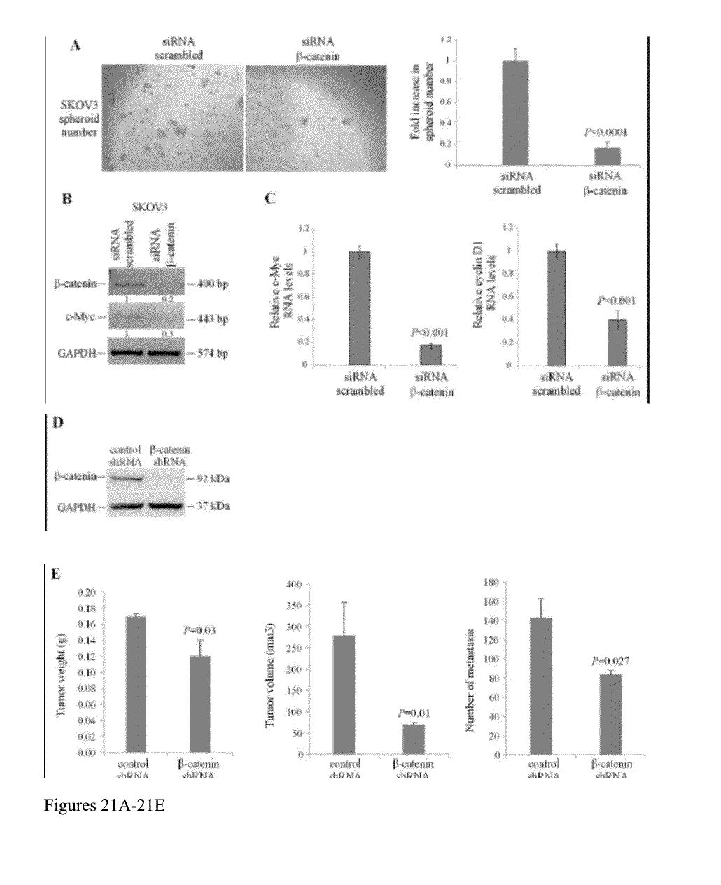

Daidzin is a strong inhibitor of ALDH2 but it also inhibits ALDH1A1 [47]. Daidzin binds in a similar location to CM026, but comparison of the structure of ALDH1A1 and ALDH2 (PDB 2VLE [48]) bound to these respective compounds shows that the two compounds bind nearly perpendicular to each other in their respective binding modes. In particular, aligning the ALDH2-daidzin structure to ALDH1A1 demonstrates that daidzin binding need not engage ALDH1A1 near its unique G458 site (FIG. 17). As discussed by Lowe et al, the tighter binding pocket of ALDH2 compared to ALDH1A1 favors more intimate interactions between the ALDH2 and daidzin, increasing specificity⁴⁸. CM026 exploits a binding pocket that is not accessible in other ALDH isoenzymes due to their larger amino acid side chains at position 458.

In contrast to CM026, the directionality of CM037 binding within the ALDH1A1 active site resembles that of daidzin in ALDH2. However, the 4-oxo group and the branched structure of CM037 near the exit of the substrate binding site exploits the same G458 region for selectivity. Our structural studies identified Gly458 as a major contributor to the selectivity of the CM026 and CM037. Sequence alignments of the other 18 human ALDH isoenzymes indicate that the only other family member with a glycine in this position is ALDH16A1. However, the function of ALDH16A1 is unknown and has a three residue deletion in the active site loop which eliminates the conserved Cys nucleophile, suggesting this protein may have functions that are independent of aldehyde dehydrogenase activity [49]. The presence of a non-glycine residue at this position does not adversely affect catalytic activity toward small substrates or inhibition by the non-selective compounds, DEAB and CM302. Consequently, these compounds identified via an esterase-based high throughput screen successfully exploited a unique structural feature found primarily in primate ALDH1A1 enzymes, which further validates the use of the esterase activity as a screening tool for ALDH isoenzymes [39].

### Example 3

### Methods of Treating Ovarian Cancer with Inhibitors of ALDH1A1

Epithelial ovarian cancer (OC) is the most lethal of all gynecologic malignancies; with the majority of cases being diagnosed at an advanced stage. Ovarian cancer metastasis is the primary cause of clinical complications and is characterized by several unique features. While in other epithelial tumors breakdown of the basement membrane is required for tumor invasion into lymphatics or vasculature and subsequent dissemination of cancer cells to distant sites, hematogenous metastasis is uncommon in ovarian cancer. Tumor dissemination occurs directly in the peritoneal cavity; with most sites of secondary implants involving the mesentery, omentum, and bowel. This is facilitated by the fact that ovarian cancer cells at the primary site are in direct anatomic contact with the overlying peritoneal surface and fluid. Their dislodgement from the primary tumor on the surface of the ovary or fallopian tube, allows cells to float in the peritoneal fluid. Importantly, after exfoliation from the primary tumor, ovarian cancer cells form multicellular aggregates or spheroids. These 3D cell aggregates serve as the vehicle for dissemination in the peritoneal cavity, protecting cells from anoikis induced by stress in the extracellular compartment.

Within spheroid structures, cells adopt mesenchymal features that are regulated by cytokines and growth factors such as estrogen, TGF-β, or other proteins secreted in the peritoneal milieu. The mesenchymal phenotype allows cells to invade when they come in contact with the mesothelium, leading to the establishment of peritoneal implants. We hypothesized that cells forming spheres are enriched in cancer stem cells (CSCs), allowing development of distant metastases and persistence after chemotherapy.

Recent reports suggest that cells grown in 3D structures behave differently compared to monolayer cultures and represent a better approximation of tumors developing in vivo. For instance, spheroids display distinct genetic expression profiles, specific intercellular signaling, and are subjected to different mechanical forces compared to monolayers. The cellular dimensionality and the resulting microenvironment exert a critical influence on cell survival, impacting drug sensitivity or resistance. Ovarian cancer spheroids can beisolated directly from malignant ascites or cultured from ovarian cancer cells by using non-adherent conditions or the hanging drop culture method. In this example, we identify oncogenic pathways regulating formation of multicellular aggregates with the goal of identifying novel targets enriched in 3D models. We now show that inhibition of such targets would disrupt spheroid formation and block cancer metastasis.

Microarray analysis comparing ovarian cancer multicellular structures to monolayers cultures identified ALDH1A1, a known CSC marker, upregulated in spheroids. ALDH1A1 was part of a gene network with β-catenin and chromatin immunoprecipitation (ChIP) demonstrated that ALDH1A1 is a direct β-catenin target. Both β-catenin knock-down and a novel ALDH1A1 inhibitor (CM037) prevented multicellular aggregation, supporting that inhibition of this pathway effectively disrupts spheroid formation.

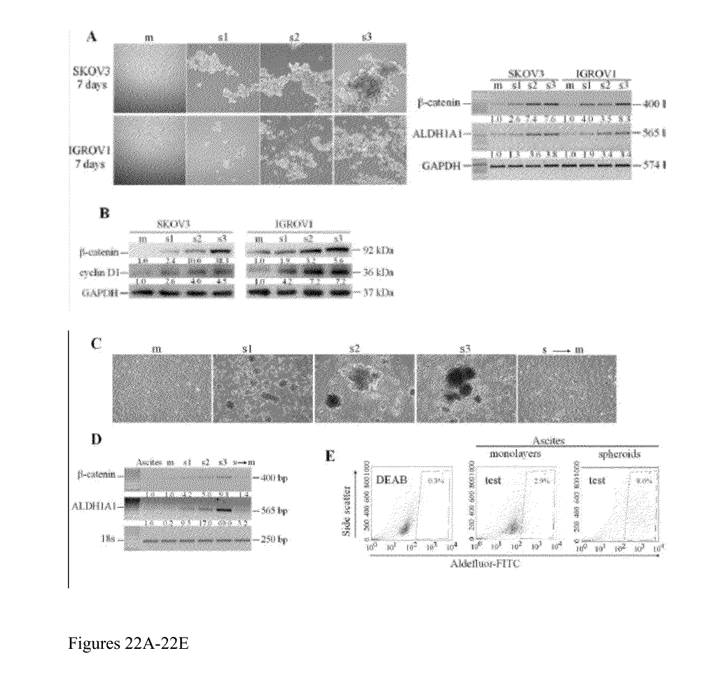

Gene Expression Profiles of ovarian cancer spheroids: ovarian cancer cell lines (IGROV1, SKOV3, A2780) and primary ovarian cancer cells derived from malignant ascites were grown as monolayers, spheroids, or transitioned back from spheroid-to-monolayer cultures. When grown under non-adherent conditions, ovarian cancer cells formed 3D aggregates with distinct features. For instance, SKOV3 adenocarcinoma cells, formed glandular structures with prominent extracellular matrix secretion; IGROV1 endometrioid cells formed branching spheroids, while A2780 and OVCA primary cells formed compact round multi-cellular aggregates. Spheroids derived from human primary cells displayed calcifications, similar to psammoma bodies formed in human tumors (FIG. 19A). To identify genes and pathways upregulated in spheroids, we compared expression profiles of monolayers, spheroids, or spheroid-to-monolayer IGROV1 cultures using Affymetrix microarrays. Unsupervised hierarchical clustering demonstrated distinct profiles of 3D versus 2D cultures, with reversal of the spheroid genotype when cells were transitioned back to monolayers (FIG. 19B) and ANOVA-based statistical analysis identified 473 transcripts differentially expressed in spheroids compared to monolayers (p<0.01 and FDR<0.01). Of those, 15 transcripts were upregulated >4 fold and 25 transcripts were downregulated >4-fold in spheroids versus monolayers (Tables 7 and 8). Validation of top differentially expressed genes using semi-quantitative RT-PCR confirmed up-regulation of aldehyde dehydrogenase 1 A1(ALDH1), angiopoietin-like 2 (ANGPTL2), thrombospondin type I (THSD), and neurotensin (NTS) and downregulation of family 25, member B (FAM25B), v-ets erythroblasosis virus homolog (ETS) in spheroids compared to monolayer cultures (FIG. 19C).

TABLE 7
Genes preferentially expressed in OC monolayers compared to spheroids (>4.0)

| | | | Fold- | |
|---|---|---|---|---|
| | | | change | p-value |
| Probeset | Gene | | (mono vs | (mono vs |
| ID | Symbol | Gene_assignment | sphere) | sphere) |
| 7951271 | MMP1 | matrix metallopcptidase 1 (interstitial | 19.07 | 1.54E−06 |
| | | collagenase) | | |
| 7933423 | FAM25B | family with sequence similarity 25, | 14.55 | 1.31E−08 |
| | | member B | | |
| 8037205 | CEACAM1 | carcinoembryonic antigen-related cell | 12.62 | 8.81E−09 |
| | | adhesion molecule 1 | | |
| 7971461 | LCP1 | lymphocyte cytosolic protein 1 (L- | 8.93 | 5.16E−08 |
| | | plastin) | | |
| 7930498 | ACSL5 | acyl-CoA synthetase long-chain family | 8.72 | 2.87E−08 |
| | | member 5 | | |
| 7928429 | PLAU | plasminogen activator, urokinase | 7.62 | 1.46E−10 |
| 8015337 | KRT15 | keratin 15 | 7.08 | 3.08E−05 |
| 8134564 | MYH16 | myosin, heavy chain 16 pseudogene | 6.79 | 2.74E−06 |
| 8051322 | XDH | xanthine dehydrogenase | 6.59 | 9.41E−07 |
| 7983215 | SQRDL | sulfide quinine reductase-like (yeast) | 6.38 | 6.38E−11 |
| 8015060 | KRT24 | keratin 24 | 5.93 | 2.62E−08 |
| 8148184 | FAM83A | family with sequence similarity 83, | 5.86 | 1.61E−07 |
| | | member A | | |
| 7952601 | ETS1 | v-ets erythroblastosis virus E26 | 5.67 | 1.72E−10 |
| | | oncogene homolog 1 (avian) | | |
| 7964834 | CPM | carboxypeptidase M | 5.64 | 1.19E−08 |
| 7961075 | CD69 | CD69 molecule | 5.21 | 2.45E−07 |
| 8135069 | SERPINE1 | serpin peptidase inhibitor, member 1 | 4.82 | 7.03E−09 |
| 8086517 | CDCP1 | CUB domain containing protein 1 | 4.60 | 3.46E−06 |
| 8095680 | IL8 | interleukin 8 | 4.45 | 2.14E−05 |
| 7970676 | SHISA2 | shisa homolog 2 (Xenopus laevis) | 4.45 | 8.25E−06 |
| 8026490 | UCA1 | urothelial cancer associated 1 (non- | 4.39 | 8.68E−06 |
| | | protein coding) | | |
| 8054712 | IL1A | interleukin 1, alpha | 4.35 | 6.79E−06 |
| 7996264 | CDH5 | cadherin 5, type 2 | 4.29 | 2.32E−06 |
| 7946292 | CYB5R2 | cytochrome b5 reductase 2 | 4.17 | 1.31E−08 |
| 8072328 | SEC14L2 | SEC14-like 2 | 4.16 | 5.48E−09 |
| 8064904 | FERMT1 | fermitin family member 1 | 4.13 | 3.17E−05 |

TABLE 8
Genes preferentially expressed in OC spheroid compared to monolayers (>4.0)

| | | | Fold- | |
|---|---|---|---|---|
| | | | change | p-value |
| Probeset | Gene | | (mono vs | (mono vs |
| ID | Symbol | Gene_assignment | sphere) | sphere) |
| 7957458 | NTS | Neurotensin | 15.41 | 7.64E−09 |
| 8141094 | PDK4 | pyruvate dehydrogenase kinase, isozyme 4 | 11.61 | 1.84E−08 |
| 8138231 | THSD7A | thrombospondin type I, domain containing | 8.24 | 1.59E−07 |
| | | 7A | | |
| 8164200 | ANGPTL2 | angiopoietin-like 2 | 7.05 | 3.89E−09 |
| 7963054 | TUBAIA | tubulin, alpha la | 6.19 | 4.28E−07 |
| 7958262 | TCP11L2 | t-complex 11, testis-specific-like 2 | 5.48 | 3.38E−05 |
| 8095110 | KIT | v-kit Hardy-Zuckerman 4 feline sarcoma | 5.42 | 2.12E−10 |
| | | viral oncogene homolog | | |
| 7968678 | FREM2 | FRAS1 related extracellular matrix protein 2 | 5.20 | 2.63E−05 |
| 8108716 | PCDHB16 | protocadherin beta 16 | 5.10 | 4.50E−10 |
| 8124492 | HIST1H2BK | histone cluster 1, H2bk | 4.89 | 5.24E−06 |
| 8161755 | ALDH1A1 | aldehyde dehydrogenase 1 family, member | 4.45 | 9.69E−09 |
| | | A1 | | |
| 7962455 | NELL2 | NEL-like 2 | 4.37 | 5.08E−08 |
| 8131550 | SCIN | Scinderin | 4.24 | 1.18E−07 |
| 8108744 | PCDHB14 | protocadherin beta 14 | 4.15 | 1.10E−06 |
| 8142997 | PLXNA4 | plexin A4 | 4.04 | 1.42E−07 |

ALDHA1 Expression in Spheroids. One of the upregulated transcripts in ovarian cancer spheroids was ALDH1A1, a recently recognized stem cell marker [29]. Semiquantitative RT-PCR validated increased mRNA expression levels of ALDH1A1 in SKOV3 and IGROV1 spheroids compared with monolayers (FIG. 19D) and flow cytometry analysis showed an increased percentage of Aldefluor-positive cells in spheroids compared with monolayers: 25.05% vs 6.68% for SKOV3 and 20.46% vs 48.0% for IGROV1 cells (FIG. 19E). The results indicate that culture under ultra-low adherent conditions selected for a population enriched in Aldefluor positive cells which are known to possess self-renewal properties [29, 30].

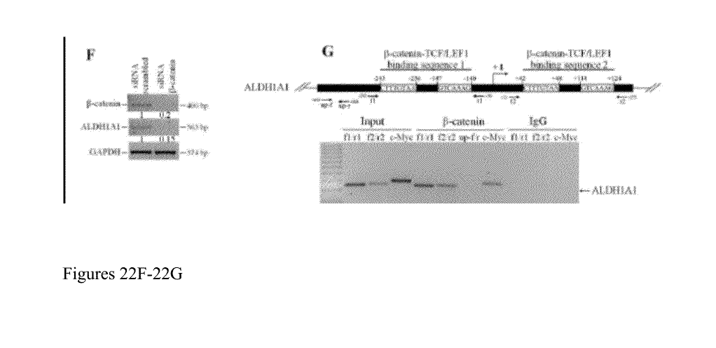

To investigate potential gene interactions in spheroid formation, the bioinformatics tool from IPA was used and identified 62 gene networks with statistically significant scores. The top gene networks represented in spheroids compared to monolayer cultures included: cancer, cellular growth and proliferation, gastrointestinal disease, cell death and survival, organismal survival, cellular development, cell cycle, and cellular movement (FIG. 20A, ranking scores ranging from 16 to 36). Within the top networks (cancer and cellular growth and proliferation) ALDH1A1 was a central node directly connected to β-catenin, c-myc, nuclear protein transcriptional regulator 1 (NUPR1), T cell factor (TCF), hepatocyte nuclear factor 4a (HNF4A), and the enhancer binding protein CEBPB (FIG. 20B), suggesting interactions with these molecules. Because of the previously recognized role of the β-catenin pathway in the maintenance of CSCs (31-33), the role of ALDH1A1 as a CSC marker (29, 34), and the observed network connections between ALDH1A1 and several of the β-catenin pathway key elements (β-catenin, c-myc, TCF, CEBPB), we focused subsequent analyses on validating the β-catenin-ALDH1A1 interaction in the generation of ovarian cancer spheroids.

B-Catenin Signaling Regulates Spheroid Formation and Self-Renewal.

Semi-quantitative RTPCR validated upregulation of β-catenin and of its target genes c-myc and cyclin D1 in IGROV1 and SKOV3 spheroids compared to monolayers (FIG. 20C). The TCF/LEF reporter assay was used to measure β-catenin transcriptional activity in spheroids vs. monolayers. A greater than 3-fold increase in TCF/LEF activity was noted in SKOV3 spheroids compared to monolayers (FIG. 20D), and a lesser magnitude, but still significant increase was recorded in IGROV1 cells grown as multi-cellular aggregates (FIG. 20D). The results indicate that the increase in spheroids proliferation was accompanied by an active β-catenin/TCF-signaling.

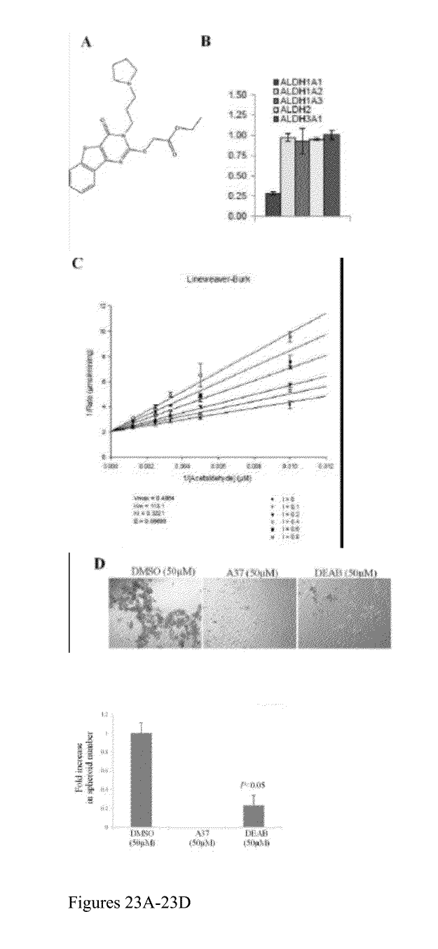

To further assess the role of β-catenin signaling in spheroid formation, SKOV3 cells were transiently transfected with siRNA targeting β-catenin or scrambled siRNA prior to plating in ultra-low adherent plates to allow sphere formation. Phase contrast microscopy showed that β-catenin knock down prevented aggregation of cells as spheroids (FIG. 21A). Semiquantitative RT-PCR analysis confirmed β-catenin knock-down in SKOV3 spheroids transfected with siRNA targeting β-catenin and corresponding downregulation of the target c-Myc compared with control (FIG. 21B). Real-time PCR analysis demonstrates decrease in mRNA expression levels of c-Myc and cyclin D1 in SKOV3 spheroids derived from cells transfected with β-catenin siRNA compared with those transfected with scrambled siRNA (FIG. 21C). These data support that β-catenin/TCF signaling regulates spheroids proliferation.

Because of the presumed role of spheroids as vehicles of ip dissemination and the observed effect of β-catenin knock down disrupting multicellular aggregation, we next measured the effects of β-catenin knock down on tumor formation and dissemination in an ip xenograft model. For this, SKOV3 cells were stably transduced with control or β-catenin targeting shRNA. Decreased β-catenin expression levels were confirmed in SKOV3 cells transduced with shRNA targeting β-catenin compared to control shRNA (FIG. 21D). Tumor volumes (278.8 mm3 vs. 69.5 mm3, p=0.01) and number of peritoneal implants (143 vs. 84, p=0.002) were significantly decreased in xenografts derived from SKOV3 cells transduced with shRNA targeting β-catenin compared to those transduced with control shRNA (FIG. 21F). Collectively, these data support the role of β-catenin in ovarian cancer spheroid formation contributing to peritoneal dissemination.

ALDH1A1 is a β-Catenin Target Gene.

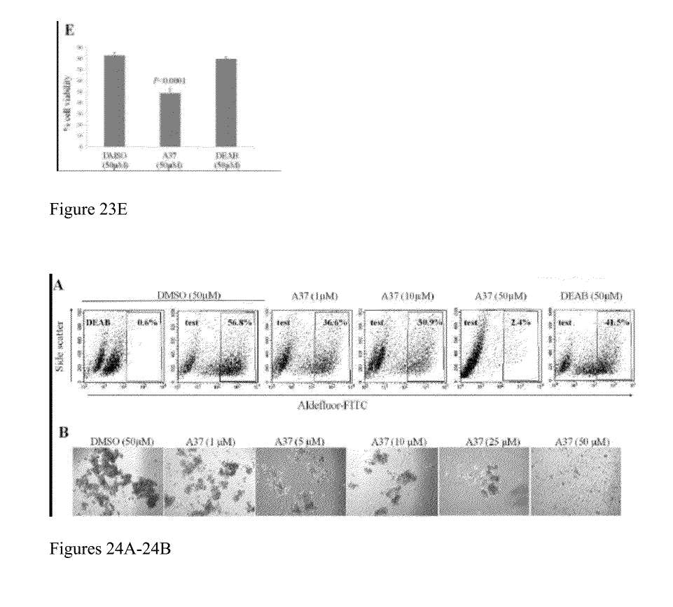

Further confirmation of β-catenin and ALDH1A1 expression used three sequential spheroids passages (s1-s3). Phase contrast microscopy demonstrated that ovarian cancer cells formed more rapidly compact and large 3D structures after enzymatic dissociation and passage through several generations (FIG. 22A). The spheroids' self-renewing and growth corresponded to an increase in β-catenin gene expression. Compared with monolayer cultures, β-catenin expression levels were up-regulated 2.6- and 4.0-fold respectively in SKOV3 and IGROV1 first generation spheroids. Further increase in β-catenin mRNA and protein levels was observed during the second and third spheroid generations in both cell lines (FIGS. 22B-C), suggesting its role in the self-renewal and maintenance of spheroids.

Furthermore, cyclin D1 expression levels, a β-catenin target (35, 36), were also increased in spheroids vs. monolayers and in subsequent spheroid passages. In parallel, ALDH1A1 mRNA expression levels increased during successive spheroid generations from ˜1.3- and 1.9-fold for the first generation to 3.8- and 3.4-fold for third generation spheroids in SKOV3 and IGROV1 cells, respectively compared with monolayers (FIG. 22B).

After establishing the importance of the β-catenin/ALDH1A1 gene interaction during the formation of spheroids from ovarian cancer cell lines, we tested whether these genes also regulate the formation of native spheroids. For this purpose, we isolated spheroids from human ovarian cancer ascites and maintained them in non-adherent conditions or as monolayers (n=5 specimens). To test β-catenin and ALDH1A1 expression during successive spheroid generations, we used enzymatic and mechanical dissociation prior to passage every 7 days. FIG. 22C illustrates increasing number and more compact spheres formed with each successive passage. The basal ALDH1A1 and β-catenin mRNA expression levels increased through successive generations compared with monolayers and were repressed when spheroids were re-plated as monolayers (FIG. 22D), supporting the significance of this pathway to multicellular aggregation in human primary cells.

Flow cytometry analyzed intracellular ALDH1A1 enzymatic activity in monolayers and spheroids derived from cells isolated from ovarian cancer ascites, noting ˜8.7% vs. 2.9% aldefluor positive cells in spheroids vs. monolayer cultures, respectively, consistent with observations in cell lines (FIG. 22E).

The direct correlation between β-catenin and ALDH1A1 expression levels observed in spheroids along with the IPA network analysis suggested that ALDH1A1 may represent a β-catenin target. To test this hypothesis, we measured ALDH1A1 mRNA expression levels after β-catenin knock down. SiRNA mediated β-catenin downregulation induced decreased ALDH1A1 expression, suggesting that ALDH1A1 is transcriptionally regulated by f3-catenin (FIG. 22F). To definitively demonstrate this concept we searched and identified potential TCF/LEF responsive elements at positions (−243 to −236), (−147 to −140), (+42 to +48) and (+118 to +124) within the ALDH1A1 promoter sequence (FIG. 22G) by using a promoter motif searching software (PROMO). ChIP tested whether β-catenin interacts with the ALDH1A1 promoter. PCR amplified the ALDH1A1 promoter fragments corresponding to the TCF/LEF responsive regions in the chromatin pulled down by a β-catenin antibody (FIG. 22G, lanes 4-5). Specificity of β-catenin antibody binding to ALDH1A1 promoter was demonstrated by observing no PCR product in chromatin immunoprecipitated with IgG (FIG. 22D, lanes 8-10). These data demonstrate that ALDH1A1 is a direct β-catenin target in ovarian cancer cells.

Targeting ALDH1A1 Disrupts Ovarian Cancer Spheroid Formation.

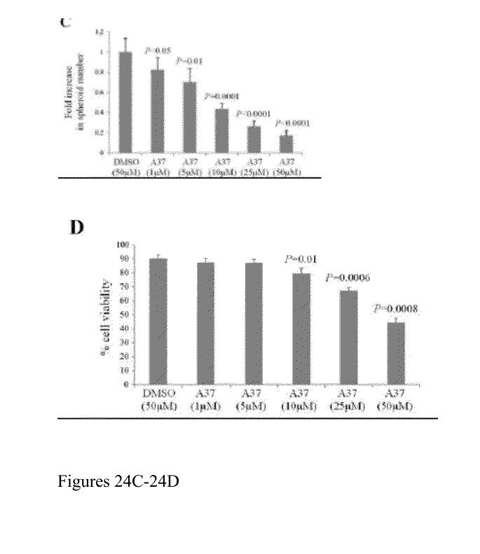

Having shown that ovarian cancer multicellular aggregates are enriched in ALDH1A1+ cells and that ALDH1A1 is a direct β-catenin target, we next explored whether ALDH1A1 enzymatic inhibitors disrupt spheroid formation and ovarian cancer cell survival under non-adherent culture conditions. We used a novel ALDH1A1 inhibitor (CM037) identified through high throughput screening of the ChemDiv library and the less selective inhibitor DEAB. CM037 has a molecular weight of 431.6 Daltons and no structural similarity to any known aldehyde dehydrogenase inhibitors (FIG. 23A). CM037 has good potency (IC50 of 4.6±0.8 μM; Ki of 300±26 nM) and is selective for ALDH1A1 with no effect on the other members of the ALDH1A subfamily (ALDH1A2 and ALDH1A3), or toward ALDH2 and ALDH3A1 at concentrations up to 100 μM (FIG. 23B). CM037 has a competitive mode of inhibition with respect to varied substrate acetaldehyde (FIG. 23C).

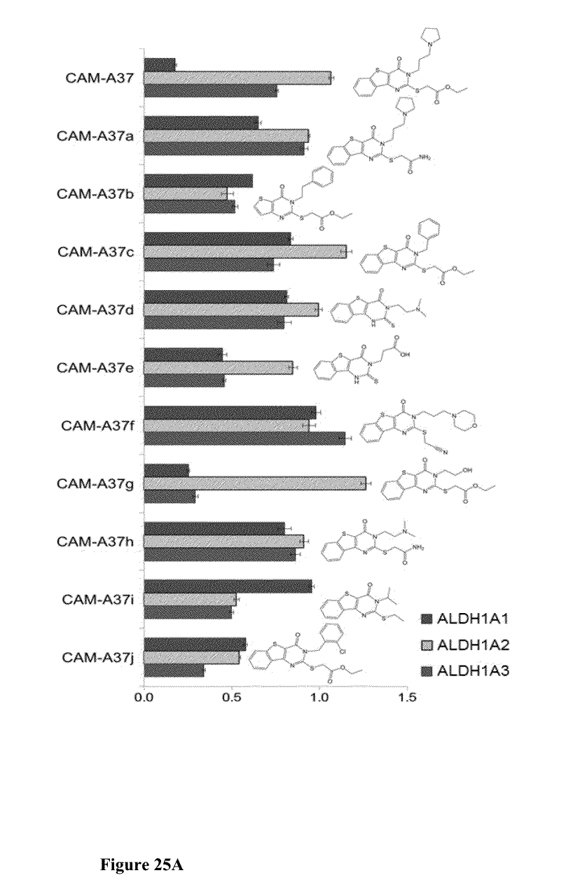

To test the activity of CM037 in ovarian cancer cells, IGROV1 cells grown under low attachment conditions were used. Under these conditions, the ALDH1A1+ population represents 30-50% of the cell population. Formation of spheres was completely inhibited by CM037 at 50 μM concentration, as well as by the nonspecific inhibitor, DEAB (FIG. 23D). The percentage of viable cells, as measured by trypan blue exclusion staining was also significantly decreased by CM037, but not by DEAB (FIG. 23E). To demonstrate that CM037 blocks ALDH1A1 function in OC cells, Aldefluor activity was measured by flow cytometry in IGROV1 spheroids treated with CM037 or DEAB for 3 days. Treatment with CM037 blocked ALDH1A1 activity in IGROV1 cells in a dose dependent manner and more significantly than DEAB (FIG. 24A). A dose response experiment also demonstrated dose-dependent inhibition of spheroid formation starting at 1 μM concentration (FIG. 24B) and a dose-dependent decrease in cell viability at concentrations greater than 10 μM (FIG. 24C). Collectively these data support that targeting of ALDH1A1 with a novel small molecule blocks ovarian cancer cell proliferation and survival under 3D culture conditions.

Through genomic profiling we identified ALDH1A1 and β-catenin signaling, two known pathways regulating CSCs, as being upregulated and interconnected in ovarian cancer spheroids compared to monolayer cultures. We demonstrated that ALDH1A1 is a direct β-catenin target and that its inhibition by novel small molecules disrupts formation of multicellular aggregates. Our data point to novel pathways activated in anoikis-resistant spheroids and potential new strategies to target them. These results have several implications.

First, we identified a specific gene network activated under 3D conditions compared to monolayer cultures. Other studies have explored the molecular characteristics of 3D cultures, demonstrating differences in gene expression based on culture conditions. It is increasingly accepted that multicellular aggregates are a better representation of human tumors compared to standard cultures on plastic. Cell growth under 3D conditions replicates the mechanical forces and gradients of oxygen and nutrient existent in native tissues which regulate cellular polarity, differentiation, and activate various morphogenic signaling programs. Well established breast cancer 3D models have replicated the architecture of normal or transformed mammary tissue and helped understanding cellular differentiation and response to therapy. Ovarian cancer cells grown as spheroids display distinct response to chemotherapeutics compared to monolayers and adopt an invasive phenotype characterized by a TGF-β fibrotic response that may protect them from unfavorable external conditions. These features strongly suggest that cells grown as spheroids are enriched in stem cells. The genomic signature proposed by our studies identifies several CSCs markers upregulated in spheroids, including KIT, β-catenin, and ALDH1A1.

Second, our data point to β-catenin as an important pathway activated in spheroids. B-catenin activation has been implicated in the self-renewal and survival of hematopoietic, cutaneous and gastrointestinal stem cells, however its role in ovarian CSCs has not yet been defined. The wingless pathway, which is an upstream regulator of β-catenin, is required for the maintenance of somatic stem cells in the Drosophila ovary and activation of β-catenin has found downstream of the stem cell factor receptor in c-kit positive ovarian tumor initiating cells. The data presented here identify β-catenin involvement in formation of ovarian cancer multi-cellular aggregates and demonstrate that its targeting disrupts sphere formation, cell proliferation under non-adherent conditions, tumor metastasis in vivo, and expression of the stem cell marker ALDH1A1. These results suggest that inhibition of this pathway, or of its key downstream elements, may be instrumental in eliminating the stem cell population.

Third, we identified ALDH1A1 upregulation in spheroids through a non-biased genome mining approach. ALDH1A1 is a member of the highly conserved ALDH family which includes 18 other enzymes involved in the metabolism of reactive aldehydes. Through their detoxification functions, ALDHs exert cytoprotective roles in various tissues. In addition, the enzymes catalyze retinol oxidation to retinal, a limiting step during the synthesis of retinoic acid, which regulates cellular differentiation. Recent reports have linked ALDHs, and particularly ALDH1A1, to stem cells, both in normal tissues, and in malignancy. It remains unknown whether the enzyme is only a CSC marker or whether it is functionally implicated in their regulation. While several other markers have been proposed for ovarian cancer stem cells, ALDH1A1 activity detectable through the aldefluor assay has been proposed and validated as a CSC phenotype. ALDH1A1+ cells have tumor initiating capacity, are resistant to cisplatin, and express upregulated levels of stem cell transcription factors (Sox2, nanog).

Here we show that ALDH1A1 expression is increased in spheroids versus monolayers and in successive spheroid generations, consistent with the recognized capacity of stem cells to organize as spheres and to self-renew. Furthermore we demonstrate that ALDH1A1 is a direct target of β-catenin, a pathway required in CSCs' self-renewal. This is the first demonstration that ALDH1A1 expression is regulated by the TCF/LEF transcriptional complex and our observations further strengthen the connection between the enzyme and an ovarian CSC phenotype.

Furthermore, we describe for the first time the activity of a new small molecule targeting ALDH1A1 in ovarian cancer spheroids. CM037 is a relatively potent first-generation selective inhibitor for ALDH1A1 with a Ki of 300 nM, without significant effect on related orthologs in the ALDH family of enzymes. The effects of this small molecule inhibitor were tested on cells growing as spheres, as the 3D culture system allows for enrichment in ALDH1A1+ population. This is the first proof of principle that selective inhibition of ALDH1A1 blocks survival of ovarian cancer cells by targeting the ALDH1A1+ population and supports a functional role for ALDH1A1 in this population. The results support further study of ALDH1A1 inhibitors in ovarian cancer models aiming to eradicate chemotherapy-resistant and perpetually self-renewing cancer stem cells.

Chemicals and Reagents.

Unless stated otherwise, chemicals and reagents were from Sigma (St Louis, Mo., USA). The antibody for cyclin D1 was from Cell Signaling Technology Inc. (Beverly, Mass.), for ALDH1A1 from Abcam (Cambridge, Mass.), for β-catenin from ECM Biosciences (Versailles, Ky., USA), and for GAPDH from Biodesign International (Saco, Me.). Secondary HRP-conjugated antibodies were from Amersham Biosciences (San Francisco, Calif.) and Santa Cruz Biotechnology Inc (Santa Cruz, Calif.). The Aldefluor kit assay was from StemCell Technologies (Vancouver, BC Canada). The ALDH1A1 inhibitor (CM037) was from ChemDiv (San Diego, Calif.), having >95% purity.

Cell Cultures.

The human ovarian cancer cell lines SKOV3, IGROV1 and A2780 were from the American Type Culture Collection (Rockville, Md.). De-identified ovarian cancer ascites samples were obtained through an IRB approved protocol of the Indiana University Simon Cancer Center Tissue Bank. Ascites tumor cells were collected by centrifugation at 200×g for 3 min. Erythrocytes were lysed by re-suspending the cell pellet in a 1:4 mixture of cold Hank's balanced salt solution modified (StemCell Technologies) supplemented with 2% FBS and red blood cell lysis buffer (0.8% ammonium chloride, 0.1 mM EDTA, pH 7.4) for 5 min. After centrifugation at 350×g for 5 min, 25,000 ascites derived tumor cells were cultured as monolayers or spheroids. SKOV3 and primary ovarian cancer cells were cultured in media containing 1:1 MCDB 105 (Sigma) and M199 (Cellgro, Herndon, Va.) supplemented with 10% FBS and antibiotics, while IGROV1 and A2780 cells were grown in RPMI 1640 at 370, under a humidified atmosphere containing 5% CO2.

Spheroid and Successive Spheroid Generation Cultures.

Ovarian cancer cell lines or primary cells were seeded at a concentration of 25,000 cells/ml in Mammocult complete medium (StemCell Technologies) and ultra-low attachment plates (Corning, Corning, N.Y.). Spheroids were trypsinized every 7 days and re-plated to generate successive generations. To observe spheroid morphology, A2780, SKOV3, IGROV1, and primary cells were cultured in a rotating bioreactor (Synthecon, Houston, Tex.) until 200-400 μM compact spheroids were visible (10-30 days). They were harvested, preserved with Histochoice MB Tissue Fixative (Amresco, Solon, Ohio) for 1 hour, and embedded in Immuno-bed resin (Polysciences, Warrington, Pa.). Two micron thick sections were cut on an ultramicrotome and stained with 0.1% methylene blue/0.15% basic fuchsin in 50% methanol.

Transfection.

Stable gene knock-down was obtained by using lentiviral transduction particles containing shRNA targeting β-catenin or shRNA control (Sigma) into SKOV3 cells. Lentiviral transduced SKOV3 cells were selected with puromycin (1.5 μM/mL). Transient transfection of short interfering RNA (siRNA) using DharmaFECT (Oz Biosciences, Marseille, France) targeted β-catenin (Dharmacon). Scrambled siRNA (Dharmacon) was used as control.

Western Blot Analysis.

Cells were lysed in ice-cold Radio-Immunoprecipitation Assay (RIPA) buffer containing protease and phosphatase inhibitor cocktail, EDTA-free (Thermo Scientific, Rockford, Ill. USA). After sonication and centrifugation, equal amounts of proteins were separated by SDS-PAGE. After electroblotting, the PVDF membranes were incubated with primary and HRP-conjugated secondary antibodies. Immunoreactive proteins were detected by enhanced chemiluminescence solution (Thermo Scientific). Images were captured by a luminescent image analyzer with a CCD camera (LAS 3000, Fuji Film) and quantified by densitometric analysis using Gel-Pro Analyzer 3.1 software.

Reverse Transcription-PCR (RT-PCR).

Total RNA was extracted using RNA STAT-60 (Tel-Test Inc., Friendswood, Tex.) and reverse-transcribed using iScript cDNA synthesis kit (Biorad). Primers and probes used for ALDH1A1, ANGPTL2, ETS1, FAM25B, NTS, NOG, THSD7A, β-catenin, cyclin D1 and c-Myc expression. The reverse transcriptase product (1 μL) and primers were heated at 94° C. for 3 min followed by 25 cycles of amplification for GAPDH and 28 cycles for the remaining genes. The RT-PCR products were separated on a 1.5% agarose gel and visualized by ethidium bromide staining under UV light. Real-time PCR was carried out on an ABI Prism 7900 platform (Applied Biosystems) using the FastStart Taqman Probe Master (Rox; Roche). The relative expression of different transcripts (cyclin D1, c-myc) was calculated as ΔCt and normalized by subtracting the Ct of target genes from that of the housekeeping control (GAPDH). Results are presented as means+/−SD of replicates. Each measurement was performed in duplicate and experiments were run three times in independent conditions.

Aldefluor Assay and Flow Cytometry.

ALDH1 enzymatic activity was measured using the Aldefluor assay (Stemcell Technologies). Briefly, dissociated monolayer and spheroid single cells were resuspended in Aldefluor assay buffer containing the ALDH1 substrate, bodipyaminoacetaldehyde (BAAA) at 1.5 mM, and incubated for 45 min at 37° C. The test ALDH1A1-positive population was gated using control cells incubated under identical condition in the presence of a 10-fold molar excess of the ALDH inhibitor, diethylamino benzaldehyde (DEAB). The relative increase in FITC signal of the ALDH-positive cells was determined by a FACS Aria II flow cytometer (BD Biosciences) and analyzed three times in independent experiments.

Gene Reporter Assays.

Dual-Luciferase Assay (Promega) was performed to quantify Wnt/β-catenin signaling through TCF/LEF1 promoter activity in SKOV3 and IGROV1 cells grown as monolayers and spheroids. Cells were transiently co-transfected with TCF/LEF1 promoter luciferase and Renilla plasmids, at a ratio of 10:1 using DreamFect Gold transfection reagent (OZ Biosciences). Luminescence was measured by using TD-20/20 Luminometer (Turner Biosystems) 24 hours after transfection. Experiments were performed in triplicate and repeated twice in independent conditions. To control for transfection efficiency, luminescence was normalized to Renilla activity.

Chromatin Immunoprecipitation (ChIP) Assay.

To detect the interaction between the transcriptional complex β-catenin/TCF/LEF1 and the ALDH1A1 promoter, we used ChIP using a kit from EMD Millipore (Billerica, Mass. USA). The DNA was extracted from β-catenin or IgG immunoprecipitates by using the QIAquick PCR purification kit (QIAGEN, Valencia, Calif.) and was subjected to PCR amplification using primers designed for the TCF/LEF1 binding domain of the ALDH1A1 promoter. The PCR products were resolved by 2% agarose-ethidium bromide gel electrophoresis, visualized by UV, and quantified by densitometric analysis using Gel-Pro Analyzer 3.1. As a positive control, DNA immunoprecipitated with β-catenin antibody was amplified using primers for c-Myc promoter, a known TCF/LEF1 target gene. As negative control, DNA immunoprecipitated with β-catenin antibody was amplified with primers designed for the ALDH1A1 promoter, upstream of the predicted TCF/LEF1 binding sites.

I.p. Ovarian Xenograft Model.

1×106 SKOV3 cells stably transduced with shRNA control or targeting β-catenin, were injected i.p. in 7-8 weeks old female nude mice (n=5 and 7, respectively) from Harlan (Indianapolis, Ind., USA). Four weeks after injection, mice were euthanized, tumors were harvested, measured bi-dimensionally if >5 mm and peritoneal implants were counted. Tumor volume was calculated as L*W2/2; where L is length and W is width.

Discovery and Characterization of ALDH Inhibitors.

ALDH1A1, ALDH1A2, ALDH1A3, ALDH2, and ALDH3A1 were produced and purified as previously described. A highthroughput screen (HTS) of 64,000 compounds from the ChemDiv Corp. was performed to identify activators and inhibitors of ALDH1A1. The hydrolysis of para-nitrophenylacetate was used as a measure ALDH1A1 activity (see SM). Selectivity for closely related orthologs was tested at 20 and 100 μM using purified recombinant human ALDH1A1, ALDH1A2, ALDH1A3, ALDH2, and ALDH3A1. Dehydrogenase activity of ALDH1A1, ALDH1A2, ALDH1A3, and ALDH2 were measured in a solution containing 100-200 nM enzyme, 200 μM NAD+, 1% DMSO, and 100 μM propionaldehyde in 50 mM sodium BES, pH 7.5. ALDH3A1 activity was measured using 25 nM enzyme, 200 μM NAD+, 1% DMSO, and 1 mM benzaldehyde in 100 mM sodium phosphate buffer, pH 7.5. All assays were performed at 25° C. and were initiated by the addition of the aldehyde substrate following a 2 minute pre-incubation with compound and NAD+. IC50 values were calculated by fitting the data to the four parameter EC50 equation using SigmaPlot (StatSys v12.3). The values represent the average of three independent experiments (each n=3) using at least two protein preparations. The mode of inhibition was determined via steady-state kinetics by co-varying inhibitor and substrate concentrations at fixed concentration of the second substrate. All data were fit to tight-binding competitive, noncompetitive, uncompetitive, and mixed inhibition models using SigmaPlot (StatSys v12.3).

Cell Proliferation and Viability.

Cell viability was measured by the trypan blue (ThermoScientific) exclusion test. Cell proliferation was quantified by the MTT assay. Spheroids were counted after centrifugation at 300×g for 5 minutes. All assays were performed in four replicates. Data are presented as means±SEM.

Gene Expression Profiling.

RNA extracted from IGROV1 cells grown as monolayer, spheroids, or spheroids transferred to monolayer, was labeled using the standard Affymetrix protocol for the Whole Transcript Target Labeling and Control Reagents kit according to the Affymetrix user manual: GeneChip® Whole Transcript Sense Target Labeling Assay GeneChip. Three biological replicates were used. Individual labeled samples were hybridized to the Human Gene 1.0 ST GeneChips® for 17 hours then washed, stained and scanned with the standard protocol using Affymetrix GCOS (GeneChip® Operating System). GCOS was used to generate CEL data files, which were imported into Partek Genomics Suite (PGS, Partek, Inc., St. Louis, Mo.).

Data Processing and Statistical Analysis.

Robust Multi-array Average (RMA) background correction with quantile normalization of data was performed to remove background noise. Genes that had a p<0.001, FDR<0.05, and fold difference exceeding +/−0.5 fold were considered dysregulated. Data are deposited in GEO under acquisition number GSE16931151.

Hierarchical clustering was performed using PGS with Pearson's dissimilarity as row and column dissimilarity measures and average linkage as linkage method. Lists of differentially expressed genes between phenotypic groups were imported into Ingenuity Pathway Analysis (©Ingenuity Systems, CA) software to identify differentially dysregulated gene pathways and networks.

It should be noted that the above description, attached figures and their descriptions are intended to be illustrative and not limiting of this invention. Many themes and variations of this invention will be suggested to one skilled in this and in light of the disclosure. All such themes and variations are within the contemplation hereof. For instance, while this invention has been described in conjunction with the various exemplary embodiments outlined above, various alternatives, modifications, variations, improvements, and/or substantial equivalents, whether known or that are or may be presently unforeseen, may become apparent to those having at least ordinary skill in the art. Various changes may be made without departing from the spirit and scope of the invention. Therefore, the invention is intended to embrace all known or later-developed alternatives, modifications, variations, improvements, and/or substantial equivalents of these exemplary embodiments.

### REFERENCES

### Example 1

1. Black, Hum Genomics, 2009. 4(2): p. 136-42.
2. Durrenberger, Parkinsons Dis, 2012. 2012: p. 214714.
3. Galter, Neurobiol Dis, 2003. 14(3): p. 637-47.
4. Ziouzenkova, Nat Med, 2007. 13(6): p. 695-702.
5. Ress, Toxicol Sci, 2003. 71(2): p. 198-206.
6. Marcato, Cell Cycle, 2011. 10(9): p. 1378-84.
7. Kastan, lood, 1990. 75(10): p. 1947-50.
8. Jones, Blood, 1995. 85(10): p. 2742-6.
9. Storms, Proc Natl Acad Sci USA, 1999. 96(16): p. 9118-23.
10. Emadi, Nat Rev Clin Oncol, 2009. 6(11): p. 638-47.
11. Dixon, Enzymes. 3d ed. 1979, New York: Academic Press. xxiv, 1116 p.
12. Rossmann, Evolutionary and Structural Relationships among Dehydrogneases, in The Enzymes, P. D. Boyer, Editor. 1975, Academic Press: New York City. p. 61-102.
13. Perez-Miller, Nat Struct Mol Biol, 2010. 17(2): p. 159-64.
14. Liu, Z. J., et al., Nat Struct Biol, 1997. 4(4): p. 317-26.
15. Farres, J., et al., Biochemistry, 1995. 34(8): p. 2592-8.
16. Wang, Biochemistry, 1995. 34(1): p. 237-43.
17. Koppaka, Pharmacol Rev, 2012. 64(3): p. 520-39.
18. Gonzalez-Segura, J Mol Biol, 2009. 385(2): p. 542-57.
19. Tsybovsky, Biochemistry, 2007. 46(11): p. 2917-29.
20. Chen, Science, 2008. 321(5895): p. 1493-5.
21. Keung, Proc Natl Acad Sci USA, 1993. 90(4): p. 1247-51.
22. Moreb, Chem Biol Interact, 2012. 195(1): p. 52-60.
23. Morgan, Chemico-Biological Interactions, 2014.
24. Hammen, Biochemistry, 2002. 41(22): p. 7156-68.
25. Perez-Miller, Biochemistry, 2003. 42(23): p. 7100-9.
26. Parajuli, B., Chem Biol Interact, 2011. 191(1-3): p. 153-8.
27. Zheng, Alcohol Clin Exp Res, 1993. 17(4): p. 828-31.
28. Otwinowski, Macromolecular Crystallography, Pt A, 1997. 276: p. 307-326.
29. Acta Crystallogr D Biol Crystallogr, 1994. 50(Pt 5): p. 760-3.
30. Emsley, Acta Crystallogr D Biol Crystallogr, 2004. 60(Pt 12 Pt 1): p. 2126-32.
31. Painter, Acta Crystallogr D Biol Crystallogr, 2006. 62(Pt 4): p. 439-50.
32. Painter, Acta Crystallogr D Biol Crystallogr, 2005. 61(Pt 4): p. 465-71.
33. Khanna, J Biol Chem, 2011. 286(50): p. 43486-94.
34. Moore, Structure, 1998. 6(12): p. 1541-51.
35. MacGibbon, Biochem J, 1978. 171(3): p. 533-8.
36. Parajuli, J Med Chem, 2014. 57(2): p. 449-61.
37. Parajuli, Chembiochem, 2014.
38. Ho, Biochemistry, 2006. 45(31): p. 9445-53.
39. Condello, Oncogene, 2014.

### REFERENCES

### Example 2

1. Vasiliou, Drug Metab Rev 2004, 36, 279-99.
2. Yoshida, Enzyme 1992, 46, 239-44.
3. Kurys, J Biol Chem 1989, 264, 4715-21.
4. Esterbauer, Free Radic Biol Med 1991, 11, 81-128.
5. Sayre, Curr Med Chem 2001, 8, 721-38.
6. Chevion, Free Radic Res 2000, 33 Suppl, S99-108.
7. Marchitti, Expert Opin Drug Metab Toxicol 2008, 4, 697-720.
8. O'Brien, Crit Rev Toxicol 2005, 35, 609-62.
9. Klyosov, Biochemistry 1996, 35, 4445-56.
10. Yoshida, Proc Natl Acad Sci USA 1984, 81, 258-61.
11. Peng, Pharmacogenet Genomics 2007, 17, 845-55.
12. Marchitti, Pharmacol Rev 2007, 59, 125-50.
13. Yao, Nat Med 2010, 16, 1024-8.
14. Rizzo, J Clin Invest 1988, 81, 738-44.
15. Rizzo, Hum Mutat 2005, 26, 1-10.
16. Mills, Nat Med 2006, 12, 307-9.
17. Geraghty, Hum Mol Genet 1998, 7, 1411-5.
18. Farrant, J Biol Chem 2001, 276, 15107-16.
19. Marchitti, Biochem Biophys Res Commun 2007, 356, 792-8.
20. Ma, Stem Cell Rev 2011, 7, 292-306.
21. Sladek, Curr Pharm Des 1999, 5, 607-25.
22. Agarwal, Adv Exp Med Biol 1995, 372, 115-22.
23. Yoon, J Hum Genet 2004, 49, 134-40.
24. Kim, Cancer Res 2005, 65, 8118-24.
25. Matsuda, Chem Res Toxicol 2006, 19, 1374-8.
26. Yokoyama, Carcinogenesis 2001, 22, 433-9.
27. Jones, Blood 1995, 85, 2742-6.
28. Chambon, FASEB J 1996, 10, 940-54.
29. Kumar, Biochim Biophys Acta 2012, 1821, 198-205.
30. Niederreither, Mech Dev 2002, 110, 165-71.
31. Koppaka, Pharmacol Rev 2012, 64, 520-39.
32. Feldman, J Biol Chem 1972, 247, 267-72.
33. Sidhu, J Biol Chem 1975, 250, 7894-8.
34. Wang, Chem Biol Interact 2009, 178, 36-9.
35. Chen, Science 2008, 321, 1493-5.
36. Parajuli, Chem Biol Interact 2011, 191, 153-8.
37. Khanna, J Biol Chem 2011, 286, 43486-94.
38. Moreb, Chem Biol Interact 2012, 195, 52-60.
39. Morgan, Chem Biol Interact 2014, In press.
40. Perez-Miller, Nat Struct Mol Biol 2010, 17, 159-64.
41. Condello, Oncogene 2014.
42. Ni, Protein Sci 1999, 8, 2784-90.
43. Srivastava, Biochemistry 1999, 38, 42-54.
44. Moore, Structure 1998, 6, 1541-51.
45. Lamb, Biochemistry 1999, 38, 6003-11.
46. Morgan, Chemico-Biological Interactions 2014, in press.
47. Keung, Proc Natl Acad Sci USA 1993, 90, 1247-51.
48. Lowe, J Med Chem 2008, 51, 4482-7.
49. Vasiliou, Chem Biol Interact 2013, 202, 22-31.
50. Levi, Blood 2009, 113, 1670-80.
51. Ucar, Chem Biol Interact 2009, 178, 48-55.
52. Landen, Mol Cancer Ther 2010, 9, 3186-99.
53. Li, Lab Invest 2010, 90, 234-44.
54. Sodek, Int J Cancer 2009, 124, 2060-70.
55. Parajuli, B. J Med Chem 2014, 57, 449-61.
56. Parajuli, Chembiochem 2014, 15, 701-12.
57. Hammen, Biochemistry 2002, 41, 7156-68.
58. Parajuli, Chembiochem 2014.
59. Zheng, Alcohol Clin Exp Res 1993, 17, 828-31.
60. Otwinowski, Z. Macromolecular Crystallography, Pt A 1997, 276, 307-326.
61. Acta Crystallogr D Biol Crystallogr 1994, 50, 760-3.
62. Emsley, P.; Acta Crystallogr D Biol Crystallogr 2004, 60, 2126-32.
63. Painter, J. Acta Crystallogr D Biol Crystallogr 2005, 61, 465-71.
64. Painter, J.; Acta Crystallogr D Biol Crystallogr 2006, 62, 439-50.

### REFERENCES

### Example 3

1. Naora, Nat Rev Cancer. 2005 May; 5(5):355-66.
2. Steeg, Nat Med. 2006 August; 12(8):895-904.
3. Levanon K, Journal of clinical oncology. 2008 Nov. 10; 26(32):5284-93.
4. Sodek K L Int J Cancer. 2008 Nov. 26.
5. Burleson K M, J Transl Med. 2006; 4:6.
6. Puiffe M L, Neoplasia. 2007 October; 9(10):820-9.
7. Burleson K M, Clin Exp Metastasis. 2004; 21(8):685-97.
8. Park S H, Mol Endocrinol. 2008 September; 22(9):2085-98.
9. Cao L, Oncogene. 2012 May 17; 31(20):2521-34.
10. Ray A, International journal of oncology. 2011 October; 39(4):797-804.
11. Moss N M, Cancer research. 2009 Sep. 1; 69(17):7121-9.
12. Fang D, Cancer Res. 2005 Oct. 15; 65(20):9328-37.
13. Zhang S, Cancer research. 2008 Jun. 1; 68(11):4311-20.
14. Cody N A L, Bmc Medical Genomics. 2008 Aug. 7; 1.
15. Chang T T, Tissue Engineering Part A. 2009 March; 15(3):559-67.
16. Gaedtke L, Journal of Proteome Research. 2007 November; 6(11):4111-8.
17. Barbone D, Journal Of Biological Chemistry. 2008 May 9; 283(19):13021-30.
18. Howes A L, Molecular Cancer Therapeutics. 2007 September; 6(9):2505-14.
19. Frankel A, Clinical Cancer Research. 2000 September; 6(9):3719-28.
20. Poland J, Electrophoresis. 2002 April; 23(7-8):1174-84.
21. Wang N, Science. 1993 May 21; 260(5111):1124-7.
22. Chen C S, Science. 1997 May 30; 276(5317):1425-8.
23. Ingber D E. Faseb Journal. 2006 May; 20(7):811-27.
24. Serebriiskii I, Matrix Biology. 2008 July; 27(6):573-85.
25. David L, Acta Biomaterialia. 2008 March; 4(2):256-63.
26. Frankel A, Cancer Research. 1997 Jun. 15; 57(12):2388-93.
27. Hazlehurst L A, Oncogene. 2003 Oct. 20; 22(47):7396-402.
28. Lengyel E, Oncogene. 2013 Aug. 12.
29. Silva I A, Cancer research. 2011 Jun. 1; 71(11):3991-4001.
30. Yasuda K, PloS one. 2013; 8(8):e68187.
31. Cai C, Mol Med Report. 2012 May; 5(5):1191-6.
32. Camper, Proc. Natl Acad. Sciences. 2011 Jul. 12; 108(28):11303-4.
33. Hoffmeyer, Science. 2012 Jun. 22; 336(6088):1549-54.
34. Shank J J, Gynecologic oncology. 2012 November; 127(2):390-7.
35. Morin P J. Bioessays. 1999 December; 21(12):1021-30.
36. Li J, BMC Cancer. 2011; 11:49.
37. Lawrenson K, BMC cell biology. 2013; 14:43.
38. Lee J M, Laboratory investigation. 2013 May; 93(5):528-42.
39. Cody N A, BMC medical genomics. 2008; 1:34.
40. Liu H, The Journal of cell biology. 2004 Feb. 16; 164(4):603-12.
41. Vidi P A, Methods in molecular biology. 2013; 945:193-219.
42. Wang F, Journal of the National Cancer Institute. 2002 Oct. 2; 94(19):1494-503.
43. Alvero, Cell Cycle. 2009 Jan. 1; 8(1):158-66.
44. L'Esperance S, BMC genomics. 2008; 9:99.
45. Sodek K L, International journal of cancer. 2009 May 1; 124(9):2060-70.
46. Sumi T, Development. 2008 September; 135(17):2969-79.
47. Wang Y, Science. 2010 Mar. 26; 327(5973):1650-3.
48. Jian H, Genes & development. 2006 Mar. 15; 20(6):666-74.
49. Song X, Xie T. Development. 2003 July; 130(14):3259-68.
50. Chau W K, Oncogene. 2013 May 30; 32(22):2767-81.
51. Jackson B, Human genomics. 2011 May; 5(4):283-303.
52. Pierre-Louis O, Stem cells. 2009 October; 27(10):2552-62.
53. Reuben J M, European journal of cancer. 2011 July; 47(10):1527-36.
54. Hammen P K, Biochemistry. 2002 Jun. 4; 41(22):7156-68.
55. Parajuli B, Chemico-biological interactions. 2011 May 30; 191(1-3):153-8.
56. Parajuli B, ChemBioChem. 2013 (in press).

We claim:

1. A pharmaceutical composition comprising
(a) a therapeutically effective amount of an inhibitor of aldehyde dehydrogenase 1A1 (ALDH1A1) selected from the group consisting of

| | | | |
|---|---|---|---|
| CM001 1.1 μM | 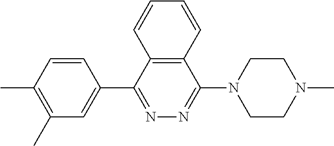 | CM045 2.5 μM | 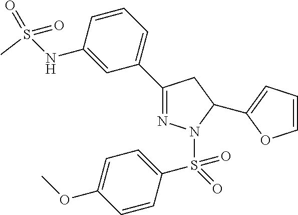 |
| CM009 5.3 μM | 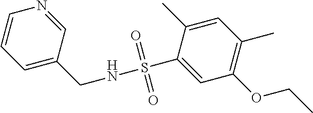 | CM047 0.31 μM | 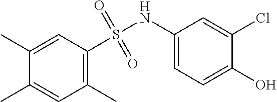 |
| CM010 1.3 μM | 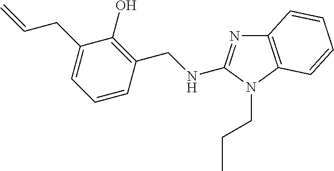 | CM053 0.21 μM | 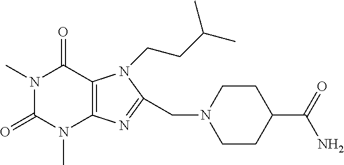 |
| CM020 0.45 μM | 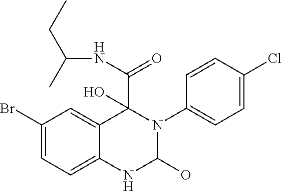 | CM055 0.24 μM | 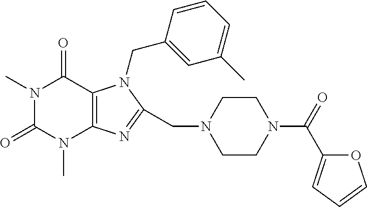 |
| CM025 2.1 μM | 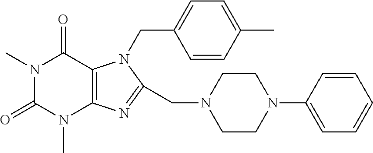 | CM056 5.4 μM | 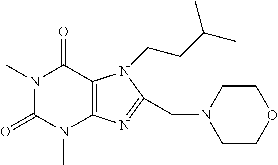 |
| CM026 0.80 μM | 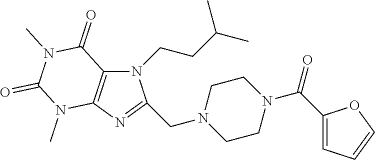 | CM057 0.92 μM | 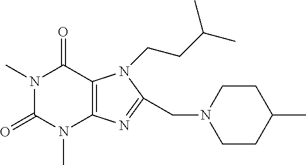 |
| CM028 2.0 μM | 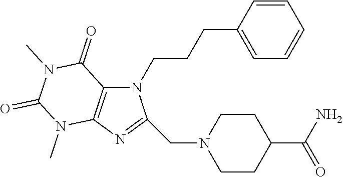 | CM302 1.1 μM | 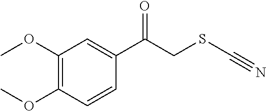 |
| CM037 4.6 μM | 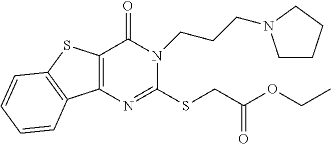 | CM306 3.5 μM | 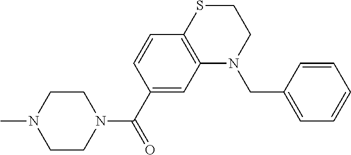 |
| CM038 0.26 μM | 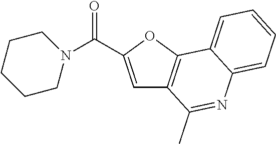 | CM307 0.57 μM | 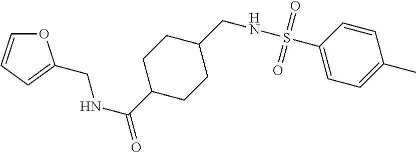 |
| CM039 0.41 μM | 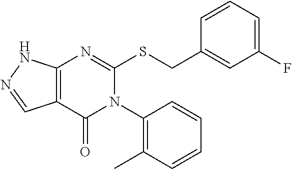 | CM037a 23 μM | 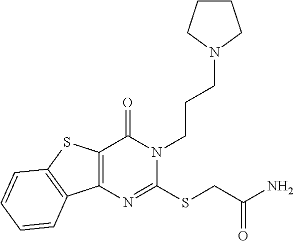 |
| CM037g 3.3 μM | 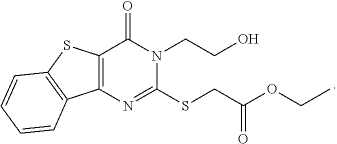 | | . |

or its pharmaceutically acceptable salt or a solvate thereof, and
(b) a pharmaceutically suitable carrier.

2. The composition of claim 1, wherein the inhibitor has substantially no does effect on ALDH2 or ALDH3A1.

3. The composition of claim 1, wherein the inhibitor is 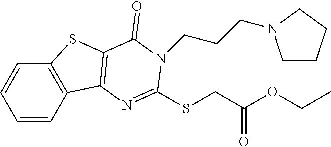 [SMILES: CCOC(=O)CSc1nc2c(sc3ccccc32)c(=O)n1CCCN1CCCC1]
(CM037) or an analog thereof.

4. The composition of claim 1, wherein the inhibitor is 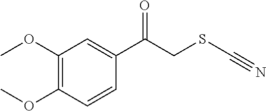 [SMILES: COc1ccc(C(=O)CSC#N)cc1OC]
(CM302) or an analog thereof.

5. The composition of claim 1, wherein the inhibitor is 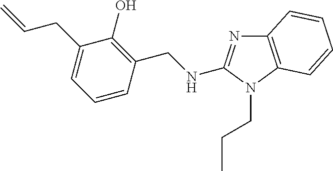 [SMILES: C=CCc1cccc(CNc2nc3ccccc3n2CCC)c1O]
(CM010) or an analog thereof.

6. The composition of claim 1, wherein the therapeutically effective amount ranges from about 0.001 μg/day/kg bodyweight to about 30 mg/day/kg bodyweight.

7. A method of treating cancer, the method comprising the step of administering a therapeutically effective amount of the composition of claim 1 to a subject in need thereof, wherein the cancer is treated.

8. The method of claim 7, wherein the cancer is selected from the group consisting of ovarian, breast and lung cancer.

9. The method of claim 7, wherein the inhibitor is formulated in an oral, topical, transdermal, parenteral, injection or infusion dosage form.

10. The method of claim 7, wherein the therapeutically effective amount ranges from about 0.001 μg/day/kg bodyweight to about 30 mg/day/kg bodyweight.

11. A kit comprising a therapeutically effective amount of at least one inhibitor of aldehyde dehydrogenase (ALDH1A1) selected from the group consisting of

| | | | |
|---|---|---|---|
| CM001 1.1 μM | 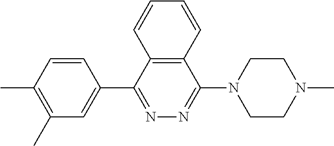 | CM045 2.5 μM | 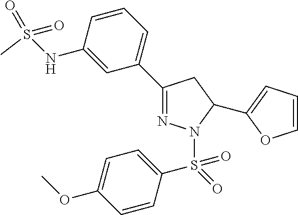 [SMILES: COc1ccc(S(=O)(=O)N2N=C(c3cccc(NS(C)(=O)=O)c3)CC2c2ccco2)cc1] |
| CM009 5.3 μM | 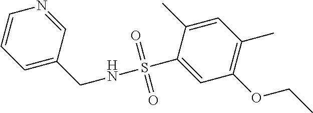 [SMILES: CCOc1cc(S(=O)(=O)NCc2cccnc2)c(C)cc1C] | CM047 0.31 μM | 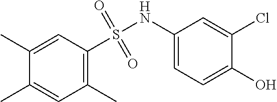 [SMILES: Cc1cc(C)c(S(=O)(=O)Nc2ccc(O)c(Cl)c2)cc1C] |
| CM010 1.3 μM | 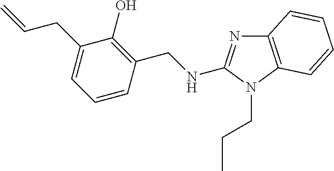 [SMILES: C=CCc1cccc(CNc2nc3ccccc3n2CCC)c1O] | CM053 0.21 μM | 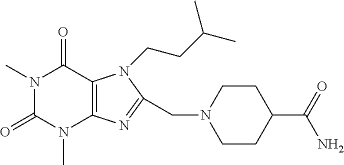 [SMILES: CC(C)CCn1c(CN2CCC(C(N)=O)CC2)nc2c1c(=O)n(C)c(=O)n2C] |
| CM020 0.45 μM | 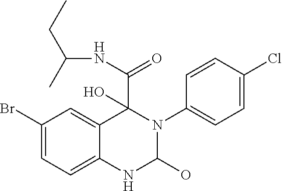 [SMILES: CCC(C)NC(=O)C1(O)c2cc(Br)ccc2NC(O)N1c1ccc(Cl)cc1] | CM055 0.24 μM | 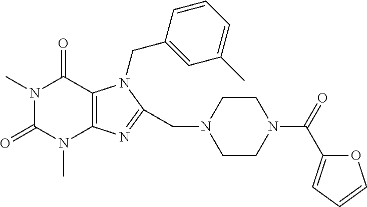 [SMILES: Cc1cccc(Cn2c(CN3CCN(C(=O)c4ccco4)CC3)nc3c2c(=O)n(C)c(=O)n3C)c1] |
| CM025 2.1 μM | 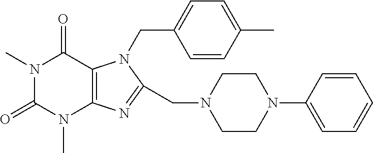 [SMILES: Cc1ccc(Cn2c(CN3CCN(c4ccccc4)CC3)nc3c2c(=O)n(C)c(=O)n3C)cc1] | CM056 5.4 μM | 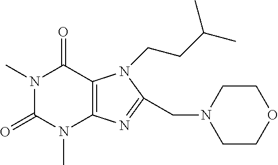 [SMILES: CC(C)CCn1c(CN2CCOCC2)nc2c1c(=O)n(C)c(=O)n2C] |
| CM026 0.80 μM | 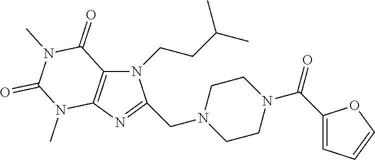 [SMILES: CC(C)CCn1c(CN2CCN(C(=O)c3ccco3)CC2)nc2c1c(=O)n(C)c(=O)n2C] | CM057 0.92 μM | 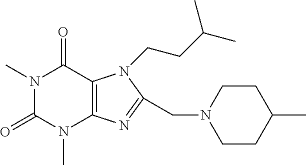 |
| CM028 2.0 μM | 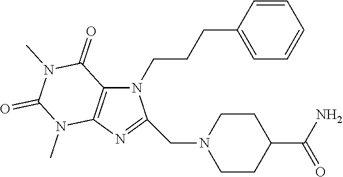 [SMILES: Cn1c(=O)c2c(nc(CN3CCC(C(N)=O)CC3)n2CCCc2ccccc2)n(C)c1=O] | CM302 1.1 μM | 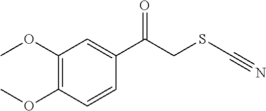 [SMILES: COc1ccc(C(=O)CSC#N)cc1OC] |
| CM037 4.6 μM | 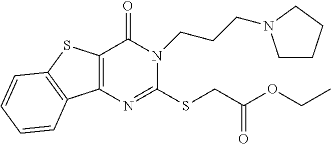 [SMILES: CCOC(=O)CSc1nc2c(sc3ccccc32)c(=O)n1CCCN1CCCC1] | CM306 3.5 μM | 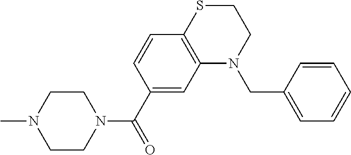 [SMILES: CN1CCN(C(=O)c2ccc3c(c2)N(Cc2ccccc2)CCS3)CC1] |
| CM038 0.26 μM | 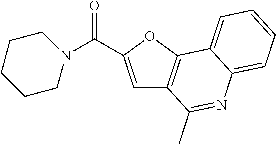 [SMILES: Cc1nc2ccccc2c2oc(C(=O)N3CCCCC3)cc12] | CM307 0.57 μM | 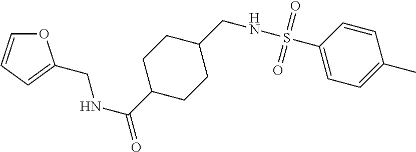 [SMILES: Cc1ccc(S(=O)(=O)NCC2CCC(C(=O)NCc3ccco3)CC2)cc1] |
| CM039 0.41 μM | 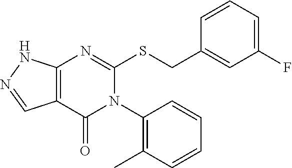 [SMILES: Cc1ccccc1-n1c(SCc2cccc(F)c2)nc2[nH]ncc2c1=O] | CM037a 23 μM | 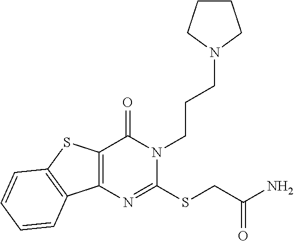 [SMILES: NC(=O)CSc1nc2c(sc3ccccc32)c(=O)n1CCCN1CCCC1] |
| CM037g 3.3 μM | 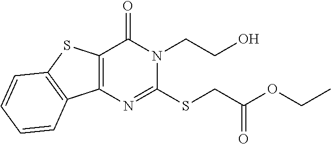 [SMILES: CCOC(=O)CSc1nc2c(sc3ccccc32)c(=O)n1CCO] | | |

or its pharmaceutically acceptable salt or a solvate thereof, and instructions for use.

12. A method of inhibiting aldehyde dehydrogenase 1A1 (ALDH1A1) in a subject, the method comprising administering a therapeutically effective amount of a composition comprising a compound selected from the group consisting of

| | | | |
|---|---|---|---|
| CM001 1.1 μM | 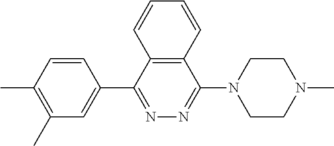 | CM045 2.5 μM | 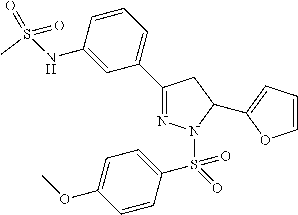 [SMILES: COc1ccc(S(=O)(=O)N2N=C(c3cccc(NS(C)(=O)=O)c3)CC2c2ccco2)cc1] |
| CM009 5.3 μM | 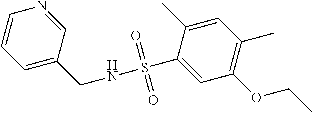 [SMILES: CCOc1cc(S(=O)(=O)NCc2cccnc2)c(C)cc1C] | CM047 0.31 μM | 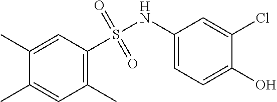 [SMILES: Cc1cc(C)c(S(=O)(=O)Nc2ccc(O)c(Cl)c2)cc1C] |
| CM010 1.3 μM | 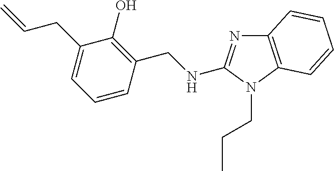 [SMILES: C=CCc1cccc(CNc2nc3ccccc3n2CCC)c1O] | CM053 0.21 μM | 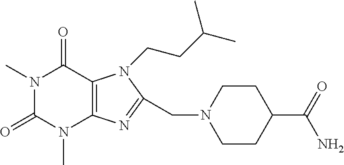 [SMILES: CC(C)CCn1c(CN2CCC(C(N)=O)CC2)nc2c1c(=O)n(C)c(=O)n2C] |
| CM020 0.45 μM | 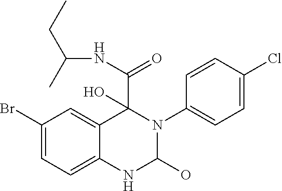 [SMILES: CCC(C)NC(=O)C1(O)c2cc(Br)ccc2NC(O)N1c1ccc(Cl)cc1] | CM055 0.24 μM | 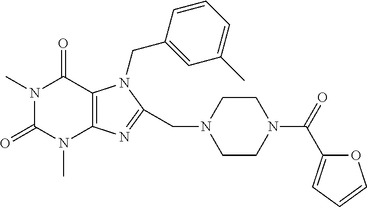 [SMILES: Cc1cccc(Cn2c(CN3CCN(C(=O)c4ccco4)CC3)nc3c2c(=O)n(C)c(=O)n3C)c1] |
| CM025 2.1 μM | 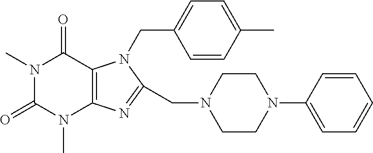 [SMILES: Cc1ccc(Cn2c(CN3CCN(c4ccccc4)CC3)nc3c2c(=O)n(C)c(=O)n3C)cc1] | CM056 5.4 μM | 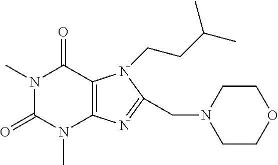 [SMILES: CC(C)CCn1c(CN2CCOCC2)nc2c1c(=O)n(C)c(=O)n2C] |
| CM026 0.80 μM | 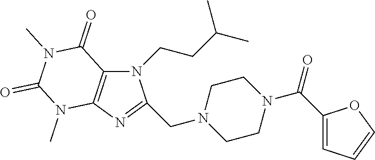 [SMILES: CC(C)CCn1c(CN2CCN(C(=O)c3ccco3)CC2)nc2c1c(=O)n(C)c(=O)n2C] | CM057 0.92 μM | 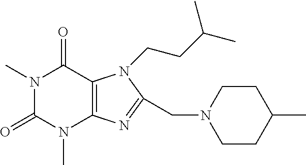 |
| CM028 2.0 μM | 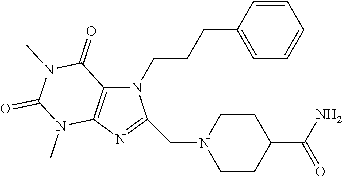 [SMILES: Cn1c(=O)c2c(nc(CN3CCC(C(N)=O)CC3)n2CCCc2ccccc2)n(C)c1=O] | CM302 1.1 μM | 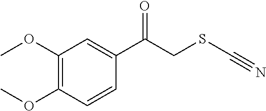 [SMILES: COc1ccc(C(=O)CSC#N)cc1OC] |
| CM037 4.6 μM | 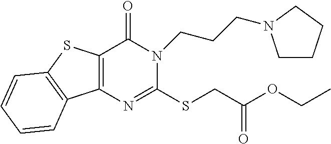 [SMILES: CCOC(=O)CSc1nc2c(sc3ccccc32)c(=O)n1CCCN1CCCC1] | CM306 3.5 μM | 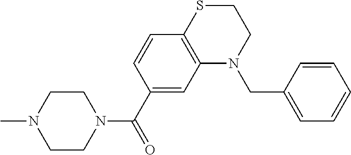 [SMILES: CN1CCN(C(=O)c2ccc3c(c2)N(Cc2ccccc2)CCS3)CC1] |
| CM038 0.26 μM | 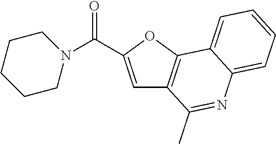 [SMILES: Cc1nc2ccccc2c2oc(C(=O)N3CCCCC3)cc12] | CM307 0.57 μM | 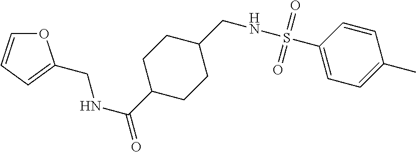 [SMILES: Cc1ccc(S(=O)(=O)NCC2CCC(C(=O)NCc3ccco3)CC2)cc1] |
| CM039 0.41 μM | 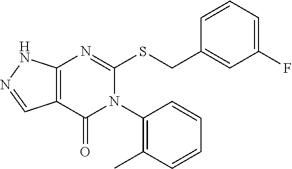 [SMILES: Cc1ccccc1-n1c(SCc2cccc(F)c2)nc2[nH]ncc2c1=O] | CM037a 23 μM | 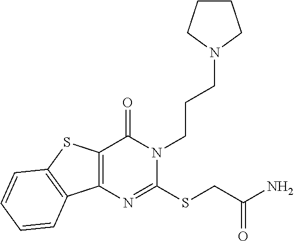 [SMILES: NC(=O)CSc1nc2c(sc3ccccc32)c(=O)n1CCCN1CCCC1] |
| CM037g 3.3 μM | 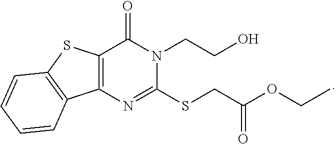 [SMILES: CCOC(=O)CSc1nc2c(sc3ccccc32)c(=O)n1CCO] | | |

or an analog thereof, and a pharmaceutically acceptable carrier to the subject, wherein the ALDH1A1 in the subject is inhibited.

13. The method of claim 12, wherein the composition has substantially no effect on ALDH2 or ALDH3A1.

14. The method of claim 12, wherein the composition is formulated in an oral, topical, transdermal, parenteral, injection or infusion dosage form.

15. The method of claim 12, wherein the therapeutically effective amount ranges from about 0.001 μg/day/kg bodyweight to about 30 mg/day/kg bodyweight.

16. The method of claim 11, wherein the composition includes a compound selected from the group consisting of CM037, CM037a, CM037g, CM302 or CM010.


SEQUENCE LISTING (21 sequences)

SEQ ID NO: 1 - DNA, 42 bp, Artificial Sequence
  forward primer - The NCBI reference sequence for ALDH1A1 (wild-type)
ctctattccaatgcatatctgaatgatttagcaggctgcatc

SEQ ID NO: 2 - DNA, 34 bp, Artificial Sequence
  the forward primer for the ALDH1A1 G458N mutant
gtgggtgaattgctataacgtggtaagtgcccag

SEQ ID NO: 3 - protein, 45 aa, Homo sapiens
TYGLAAAVFTKDLDKANYLSQALQAGTVWVNCYDVFGAQSPFGGY

SEQ ID NO: 4 - protein, 45 aa, Homo sapiens
FYGLSAGVFTKDIDKAITISSALQAGTVWVNCYGVVSAQCPFGGF

SEQ ID NO: 5 - protein, 45 aa, Homo sapiens
DFGLVAAVFTNDINKALTVSSAMQAGTVWINCYNALNAQSPFGGF

SEQ ID NO: 6 - protein, 45 aa, Homo sapiens
DYGLTAAVFTKNLDKALKLASALESGTVWINCYNALYAQAPFGGF

SEQ ID NO: 7 - protein, 45 aa, Homo sapiens
RYGLAAAVFTRDLDKAMYFTQALQAGTVWVNTYNIVTCHTPFGGF

SEQ ID NO: 8 - protein, 45 aa, Homo sapiens
EFGLASGVFTRDINKALYVSDKLQAGTVFINTYNKTDVAAPFGGF

SEQ ID NO: 9 - protein, 45 aa, Homo sapiens
EFGLASGVFTRDINKAMYVSEKLEAGTVFINTYNKTDVAAPFGGV

SEQ ID NO: 10 - protein, 47 aa, Homo sapiens
EKPLALYMFSSNDKVIKKMIAETSSGGVAANDVIVHITLHSLPFGGV

SEQ ID NO: 11 - protein, 47 aa, Homo sapiens
EKPLALYVFSHNHKLIKRMIDETSSGGVTGNDVIMHFTLNSFPFGGV

SEQ ID NO: 12 - protein, 47 aa, Homo sapiens
EKPLALYAFSNSSQVVKRVLTQTSSGGFCGNDGFMHMTLASLPFGGV

SEQ ID NO: 13 - protein, 47 aa, Homo sapiens
EKPLALYAFSNSSQVVNQMLERTSSGSFGGNEGFTYISLLSVPFGGV

SEQ ID NO: 14 - protein, 49 aa, Homo sapiens
SYGLTGAVFSQDKDVVQEATKVLRNAAGNFYINDKSTGSIVGQQPFGGA

SEQ ID NO: 15 - protein, 45 aa, Homo sapiens
DVGLAGYFYSQDPAQIWRVAEQLEVGMVGVNEGLISSVECPFGGV

SEQ ID NO: 16 - protein, 46 aa, Homo sapiens
PYGNGTAIFTTNGATARKYAHLVDVGQVGVNVPIPVPLPMFSFTGS

SEQ ID NO: 17 - protein, 48 aa, Homo sapiens
KQGLSSSIFTKDLGRIFRWLGPKGSDCGIVNVNIPTSGAEIGGAFGGE

SEQ ID NO: 18 - protein, 45 aa, Homo sapiens
KYGLAATVWSSNVGRVHRVAKKLQSGLVWTNCWLIRELNLPFGGM

SEQ ID NO: 19 - protein, 45 aa, Homo sapiens
TFGLAAGVFTRDIQRAHRVVAELQAGTCFINNYNVSPVELPFGGY

SEQ ID NO: 20 - protein, 45 aa, Homo sapiens
PRGGSASVWSERLGQALELGYGLQVGTVWINAHGLRDPSVPTGGC

SEQ ID NO: 21 - protein, 45 aa, Homo sapiens
PVGLEGLLTTKWLLRGKDHVVSDFSEHGSLKYLHENLPIPQRNTN